(19) United States
(12) Patent Application Publication
(10) Pub. No.: US 2025/0127869 A1
(43) Pub. Date: Apr. 24, 2025

(54) Tuberculosis Antigen Cassettes

(71) Applicant: International AIDS Vaccine Initiative, Inc., New York, NY (US)

(72) Inventors: Dominick Laddy, New York, NY (US); Danilo Casimiro, New York, NY (US); Thomas Evans, New York, NY (US); Megan Fitzpatrick Forrest, New York, NY (US); Nathalie Cadieux, New York, NY (US)

(21) Appl. No.: 18/936,309

(22) Filed: Nov. 4, 2024

Related U.S. Application Data
(63) Continuation of application No. 18/184,869, filed on Mar. 16, 2023, now Pat. No. 12,168,047.
Application No. 18/184,869 is a continuation of application No. 16/756,206, filed on Apr. 15, 2020, now Pat. No. 11,638,749.
(60) Provisional application No. 62/573,432, filed on Oct. 17, 2017.

(51) Int. Cl. A61K 39/04; C07K 14/35

(52) U.S. Cl. CPC A61K 39/04; C07K 14/35; C07K 2319/00

(57) ABSTRACT

The present disclosure provides fusion proteins comprising Mycobacterium tuberculosis (Mtb) antigens, nucleic acid molecules encoding the same, vectors comprising nucleic acid molecules, compositions comprising the same, and methods of eliciting an immune response against tuberculosis.

### REFERENCE TO SEQUENCE LISTING

This application includes a Sequence Listing filed electronically as an XML file named 901513173SEQ, created on Oct. 7, 2024, with a size of 122,055 bytes. The Sequence Listing is incorporated herein by reference.

### FIELD

The present disclosure is directed, in part, to fusion proteins comprising Mycobacterium tuberculosis (Mtb) antigens, nucleic acid molecules encoding the same, vectors comprising nucleic acid molecules, compositions comprising the same, and methods of eliciting an immune response against tuberculosis.

### BACKGROUND

Tuberculosis (TB) is a global health problem resulting in 8 million new cases and 2 million deaths each year. The emergence of multi-drug and totally-drug resistant strains of TB only makes this problem more severe. The life cycle of Mtb has 3 stages. In the acute phase following initial infection the bacteria replicate in the host and virulence factors are expressed, leading to the generation of an immune response by the host. As the immune response begins to control the infection, the Mtb enters a latent, asymptomatic state in which the bacteria become non-replicating and are encased in granulomas. The bacterium can persist in this latent state in infected individuals for many years, making diagnosis and treatment of disease difficult. In some cases, the bacteria are reactivated and begin replicating again, leading back to the disease state. Reactivation can occur for numerous reasons, including immune suppression caused by diseases such as HIV, treatments such as chemotherapy, or the weakening of the immune system due to aging. An estimated 2 billion people are latently infected with Mtb worldwide, and reactivation of latent Mtb accounts for most new cases of active TB disease. Reactivation is associated with inflammation, necrosis and cavitation of the lung, a process that results in draining of the lesions into the bronchus. Aerosols generated when individuals with bronchial lesions cough causes dissemination of the Mtb organism to uninfected, susceptible persons, and the transmission cycle is thus maintained.

The only currently available vaccine against TB, Mycobacterium bovis (Bacille Calmette-Guérin) (BCG), was first introduced in 1921. BCG has been widely utilized and while studies show that for some purposes BCG is effective (e.g., against disseminated TB), it is known to be ineffective with respect to preventing the development, persistence and reactivation of latent TB. There is an ongoing need to develop improved, more effective vaccines against TB. In particular, there is a need to develop vaccines that provide protection against the development, maintenance and/or reactivation of latent tuberculosis infection. With the availability of the entire genomic sequence of Mtb, and the tools for bioinformatic and experimental analysis of Mtb antigens, many new potential Mtb vaccine candidates have been identified in recent years. These include antigens that are involved in acute infection, maintenance of latency, or reactivation of Mtb. There are a range of delivery strategies in clinical development that are comprised of combinations of these and other antigens that have been tested in animal models and are currently or will soon be in clinical trials.

While vaccines are often effective to immunize individuals prophylactically or therapeutically against pathogen infection or human diseases, there is a need for improved vaccines. There is also a need for compositions and methods that produce an enhanced immune response. Likewise, while some immunotherapeutics are useful to modulate immune response in a patient, there remains a need for improved immunotherapeutic compositions and methods.

### SUMMARY

The present disclosure describes antigen cassettes (and specified variants) that can be used to create tuberculosis vaccines comprising specified Mycobacterium tuberculosis (Mtb) antigens. The disclosure also describes the strategic combination of antigens which are incorporated into a variety of delivery platforms in such a way as to provide pathways to a matrix of matched combinations of antigen delivery to obtain an optimized immune response. The subject matter described herein can be used as a prophylactic or therapeutic TB vaccine. Specific selection of antigens for inclusion into a usable cassette was based on a number of additional parameters including, for example, a thorough review of the literature, expression data, responses by human T cells, inclusion of human immunogenic regions, mouse protection studies, and conservation in sequence across most strains of TB with full genome sequences (or lack thereof for the Variable antigens).

The constructs described herein can be integrated into several delivery platforms that include the following classes (but not exhaustive) of representative delivery platforms: 1) viral vector delivery systems, 2) recombinant BCG, 3) recombinant purified protein fusions, 4) DNA plasmid vector systems, and 5) RNA vector systems. These delivery platforms can be used either in a single platform alone or in combinations as matched antigen prime-boost approaches. In addition, the use of these antigens in a single rBCG vector system, which is envisioned to be used as an antigen matched prime for a boost with any of the modalities above, including protein, viral vectors, nucleic acids, or others.

The present disclosure provides fusion proteins that comprise at least two PE Mtb antigens, at least two PPE Mtb antigens, at least two ESX Mtb antigens, or at least two variable Mtb antigens.

The present disclosure also provides nucleic acid molecules encoding fusion proteins that comprise at least two PE Mtb antigens, at least two PPE Mtb antigens, at least two ESX Mtb antigens, or at least two variable Mtb antigens.

The present disclosure also provides: compositions comprising the fusion proteins and a pharmaceutically acceptable carrier; vectors encoding the fusion proteins; compositions comprising the vectors and a pharmaceutically acceptable carrier; cells comprising the vectors; compositions comprising the cells and a pharmaceutically acceptable carrier.

The present disclosure also provides compositions that comprise at least two PE Mtb antigens, at least two PPE Mtb antigens, at least two ESX Mtb antigens, or at least two variable Mtb antigens, and a pharmaceutically acceptable carrier.

The present disclosure also provides compositions that comprise at least two Mtb antigens, wherein the composition comprises at least one nucleic acid molecule encoding at least one of the Mtb antigens, and a pharmaceutically acceptable carrier.

The present disclosure also provides methods of eliciting an immune response against Mycobacterium tuberculosis in a mammal comprising administering to the mammal an immunologically sufficient amount of one or more fusion proteins described herein.

The present disclosure also provides methods of eliciting an immune response against Mycobacterium tuberculosis in a mammal comprising administering to the mammal an immunologically sufficient amount of a composition comprising one or more fusion proteins described herein, and a pharmaceutically acceptable carrier.

The present disclosure also provides methods of eliciting an immune response against Mycobacterium tuberculosis in a mammal comprising administering to the mammal an immunologically sufficient amount of a composition comprising one or more fusion proteins described herein, and a pharmaceutically acceptable carrier, wherein the composition comprises at least one nucleic acid molecule encoding at least one of the Mtb antigens.

The present disclosure also provides any one or more of the fusion proteins described herein for use in the preparation of a medicament for treating or preventing a Mycobacterium tuberculosis infection.

The present disclosure also provides any one or more of the fusion proteins described herein for use in treating or preventing a Mycobacterium tuberculosis infection.

The present disclosure also provides use of any one or more of the fusion proteins described herein in the preparation of a medicament for treating or preventing a Mycobacterium tuberculosis infection.

The present disclosure also provides use of any one or more of the fusion proteins described herein in treating or preventing a Mycobacterium tuberculosis infection.

The present disclosure also provides compositions for use in the preparation of a medicament for treating or preventing a Mycobacterium tuberculosis infection, wherein the composition comprises any one or more of the fusion proteins described herein, and a pharmaceutically acceptable carrier.

The present disclosure also provides any one or more of the compositions described herein for use in treating or preventing a Mycobacterium tuberculosis infection, and a pharmaceutically acceptable carrier.

The present disclosure also provides any one or more of the compositions described herein in the preparation of a medicament for treating or preventing a Mycobacterium tuberculosis infection, and a pharmaceutically acceptable carrier.

The present disclosure also provides use of any one or more of the compositions described herein in treating or preventing a Mycobacterium tuberculosis infection, and a pharmaceutically acceptable carrier.

The present disclosure also provides any one or more of the compositions described herein for use in the preparation of a medicament for treating or preventing a Mycobacterium tuberculosis infection, and a pharmaceutically acceptable carrier; wherein the composition comprises at least one nucleic acid molecule encoding at least one of the Mtb antigens.

The present disclosure also provides any one or more of the compositions described herein for use in treating or preventing a Mycobacterium tuberculosis infection, and a pharmaceutically acceptable carrier, wherein the composition comprises at least one nucleic acid molecule encoding at least one of the Mtb antigens.

The present disclosure also provides use of any one or more of the compositions described herein in the preparation of a medicament for treating or preventing a Mycobacterium tuberculosis infection, and a pharmaceutically acceptable carrier, wherein the composition comprises at least one nucleic acid molecule encoding at least one of the Mtb antigens.

The present disclosure also provides use of any one or more of the compositions described herein in treating or preventing a Mycobacterium tuberculosis infection, and a pharmaceutically acceptable carrier, wherein the composition comprises at least one nucleic acid molecule encoding at least one of the Mtb antigens.

The present disclosure also provides fusion proteins, compositions, cells, vectors, methods, and uses, as described herein, substantially as described herein.

### DESCRIPTION OF EMBODIMENTS

The terminology used herein is for the purpose of describing particular embodiments only and is not intended to be limiting.

As used herein, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, “adjuvant” means any molecule added to any composition described herein to enhance the immunogenicity of the Mtb antigens.

As used herein, “coding sequence” or “encoding nucleic acid” means the nucleic acids (RNA or DNA molecule) that comprise a nucleotide sequence which encodes an Mtb antigen. The coding sequence can further include initiation and termination signals operably linked to regulatory elements including a promoter and polyadenylation signal capable of directing expression in the cells of an individual or mammal to which the nucleic acid is administered.

As used herein, “consensus” or “consensus sequence” means a polypeptide sequence based on analysis of an alignment of multiple subtypes of a particular Mtb antigen. Nucleic acid sequences that encode a consensus polypeptide sequence can be prepared. Vaccines comprising Mtb antigens that comprise consensus sequences and/or nucleic acid molecules that encode such antigens can be used to induce broad immunity against multiple subtypes or serotypes of a particular antigen.

As used herein, “electroporation” means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

As used herein, “fragment” with respect to nucleic acid sequences, means a nucleic acid sequence or a portion thereof, that encodes a portion of an Mtb antigen capable of eliciting an immune response in a mammal that cross reacts with a full length wild type Mtb antigen. The fragments can be DNA fragments selected from at least one of the various nucleotide sequences that encode protein fragments set forth below.

As used herein, “fragment” or “immunogenic fragment” with respect to polypeptide sequences, means a portion of an Mtb antigen capable of eliciting an immune response in a mammal that cross reacts with a full length wild type strain Mtb antigen. Fragments of consensus or wild type Mtb antigens can comprise at least 10%, at least 20%, at least 30%, at least 40%, at least 50%, at least 60%, at least 70%, at least 80%, at least 90% or at least 95% of a consensus or wild type Mtb antigen. In some embodiments, fragments of consensus proteins can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more of a consensus or wild type protein.

As used herein, “genetic construct” refers to the DNA or RNA molecules that comprise a nucleotide sequence which encodes an Mtb antigen. The coding sequence includes initiation and termination signals operably linked to regulatory elements including a promoter and polyadenylation signal capable of directing expression in the cells of the individual to whom the nucleic acid molecule is administered.

As used herein, “expressible form” refers to gene constructs that contain the necessary regulatory elements operable linked to a coding sequence that encodes an Mtb antigen such that when present in the cell of the individual, the coding sequence will be expressed.

As used herein, “homology” refers to a degree of complementarity. There can be partial homology or complete homology (i.e., identity). A partially complementary sequence that at least partially inhibits a completely complementary sequence from hybridizing to a target nucleic acid is referred to using the functional term “substantially homologous.” When used in reference to a double-stranded nucleic acid sequence such as a cDNA or genomic clone, the term “substantially homologous” refers to a probe that can hybridize to a strand of the double-stranded nucleic acid sequence under conditions of low stringency. When used in reference to a single-stranded nucleic acid sequence, the term “substantially homologous” refers to a probe that can hybridize to (i.e., is the complement of) the single-stranded nucleic acid template sequence under conditions of low stringency.

As used herein, “identical” or “identity” in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) residues can be considered equivalent. Identity can be performed manually or by using a computer sequence algorithm such as BLAST or BLAST 2.0.

As used herein, “immune response” means the activation of a host's immune system, e.g., that of a mammal, in response to the introduction of an Mtb antigen. The immune response can be in the form of a cellular or humoral response, or both.

As used herein, “Mtb antigen” means an antigen from Mycobacterium tuberculosis, which may be an isolated antigen, or an antigen that forms part of a fusion protein with other antigen(s).

As used herein, “nucleic acid” or “oligonucleotide” or “polynucleotide” means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions. Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

As used herein, “operably linked” means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, “promoter” means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

As used herein, “stringent hybridization conditions” means conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions can be selected to be about 5 to 10° C. lower than the thermal melting point (Tₘ) for the specific sequence at a defined ionic strength pH. The Tₘ can be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions can be those in which the salt concentration is less than about 1.0 M sodium ion, such as about 0.01 to 1.0 M sodium ion concentration (or other salts) at pH 7.0 to 8.3 and the temperature is at least about 30° C. for short probes (e.g., about 10 to 50 nucleotides) and at least about 60° C. for long probes (e.g., greater than about 50 nucleotides). Stringent conditions can also be achieved with the addition of destabilizing agents such as formamide. For selective or specific hybridization, a positive signal can be at least 2 to 10 times background hybridization. Exemplary stringent hybridization conditions include the following: 50% formamide, 5× SSC, and 1% SDS, incubating at 42° C., or, 5× SSC, 1% SDS, incubating at 65° C., with wash in 0.2× SSC, and 0.1% SDS at 65° C.

As used herein, “substantially complementary” means that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used herein, “substantially identical” means that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used herein, “variant” with respect to a nucleic acid means: i) a portion or fragment of a referenced nucleotide sequence; ii) the complement of a referenced nucleotide sequence or portion thereof; iii) a nucleic acid that is substantially identical to a referenced nucleic acid or the complement thereof; or iv) a nucleic acid that hybridizes under stringent conditions to the referenced nucleic acid, complement thereof, or a sequences substantially identical thereto.

As used herein, “variant” with respect to a peptide or polypeptide means that it differs in amino acid sequence by the insertion, deletion, or conservative substitution of amino acids, but retains at least one biological activity. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. Amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “vector” means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector.

The present disclosure provides fusion proteins comprising at least two Mtb antigens. In some embodiments, the fusion protein comprises at least three Mtb antigens. In some embodiments, the fusion protein comprises at least four Mtb antigens. In some embodiments, the fusion protein comprises at least five Mtb antigens. In some embodiments, the fusion protein comprises at least six Mtb antigens. In some embodiments, the fusion protein comprises at least seven Mtb antigens.

In some embodiments, the nucleic acid molecule encoding any particular Mtb antigen can be a mycobacterial sequence, a bacterial codon optimized sequence (such as an E. coli optimized sequence), or a mammalian optimized sequence (such as a human optimized sequence). The E. coli optimized sequences can be used, for example, to produce fusion proteins. The human optimized sequences can be used in, for example, viral vectors. Methods of codon optimization (whether for bacterial or mammalian) are well known to the skilled artisan.

In some embodiments, any of the fusion proteins described herein can comprise a C-terminal HA tag (e.g., YPYDVPDYA) (SEQ ID NO:17).

In some embodiments, the Mtb antigen is a PE antigen. In some embodiments, the PE antigen is Rv3872 (also known as PE35), Rv1788 (also known as PE18), Rv3893c (also known as PE36), Rv0285 (also known as PE5), Rv1818c (also known as PE_PGRS33; includes only the PE domain), Rv0159c (also known as PE3; includes only the PE domain), or Rv1172c (also known as PE12; includes only the PE domain).

A nucleotide sequence encoding Rv3872 is shown in Table 1 as SEQ ID NO:1, and an amino acid sequence of Rv3872 is shown in Table 1 as SEQ ID NO:2.

A nucleotide sequence encoding Rv1788 is shown in Table 1 as SEQ ID NO:3, and an amino acid sequence of Rv1788 is shown in Table 1 as SEQ ID NO:4.

A nucleotide sequence encoding Rv3893c is shown in Table 1 as SEQ ID NO:5, and an amino acid sequence of Rv3893c is shown in Table 1 as SEQ ID NO:6.

A nucleotide sequence encoding Rv0285 is shown in Table 1 as SEQ ID NO:7, and an amino acid sequence of Rv0285 is shown in Table 1 as SEQ ID NO:8.

A nucleotide sequence encoding Rv1818c is shown in Table 1 as SEQ ID NO:9, and an amino acid sequence of Rv1818c is shown in Table 1 as SEQ ID NO:10.

A nucleotide sequence encoding Rv0159c is shown in Table 1 as SEQ ID NO:11, and an amino acid sequence of Rv0159c is shown in Table 1 as SEQ ID NO:12.

A nucleotide sequence encoding Rv1172c is shown in Table 1 as SEQ ID NO:13, and an amino acid sequence of Rv1172c is shown in Table 1 as SEQ ID NO:14.

TABLE 1
PE Antigens

| Con- | nucleotide sequence |
|---|---|
| struct | amino acid sequence |
| Rv3872 | ATGGAGAAGATGAGCCACGACCCCATCGCCGCCGACATCGGCA |
| (PE35) | CCCAGGTGAGCGACAACGCCCTGCACGGCGTGACCGCCGGCAG |
| | CACCGCCCTGACCAGCGTGACCGGCCTGGTGCCCGCCGGCGCC |
| | GACGAGGTGAGCGCCCAGGCCGCCACCGCCTTCACCAGCGAGG |
| | GCATCCAGCTGCTGGCCAGCAACGCCAGCGCCCAGGACCAGCT |
| | GCACAGGGCCGGCGAGGCCGTGCAGGACGTGGCCAGGACCTA |
| | CAGCCAGATCGACGACGGCGCCGCCGGCGTGTTCGCCGAGTGA |
| | (SEQ ID NO: 1) |
| | MEKMSHDPIAADIGTQVSDNALHGVTAGSTALTSVTGLVPAGA |
| | DEVSAQAATAFTSEGIQLLASNASAQDQLHRAGEAVQDVARTY |
| | SQIDDGAAGVFAE (SEQ ID NO: 2) |
| Rv1788 | ATGAGCTTCGTGACCACCCAGCCCGAGGCCCTGGCCGCCGCCG |
| (PE18) | CCGGCAGCCTGCAGGGCATCGGCAGCGCCCTGAACGCCCAGAA |
| | CGCCGCCGCCGCCACCCCCACCACCGGCGTGGTGCCCGCCGCC |
| | GCCGACGAGGTGAGCGCCCTGACCGCCGCCCAGTTCGCCGCCC |
| | ACGCCCAGATCTACCAGGCCGTGAGCGCCCAGGCCGCCGCCAT |
| | CCACGAGATGTTCGTGAACACCCTGCAGATGAGCAGCGGCAGC |
| | TACGCCGCCACCGAGGCCGCCAACGCCGCCGCCGCCGGCTGA |
| | (SEQ ID NO: 3) |
| | MSFVTTQPEALAAAAGSLQGIGSALNAQNAAAATPTTGVVPAA |
| | ADEVSALTAAQFAAHAQIYQAVSAQAAAIHEMEVNTLQMSSGS |
| | YAATEAANAAAAG (SEQ ID NO: 4) |
| Rv3893c | ATGGTGTGGAGCGTGCAGCCCGAGGCCGTGCTGGCCAGCGCCG |
| (PE36) | CCGCCGAGAGCGCCATCAGCGCCGAGACCGAGGCCGCCGCCGC |
| | CGGCGCCGCCCCCGCCCTGCTGAGCACCACCCCCATGGGCGGC |
| | GACCCCGACAGCGCCATGTTCAGCGCCGCCCTGAACGCCTGCG |
| | GCGCCAGCTACCTGGGCGTGGTGGCCGAGCACGCCAGCCAGAG |
| | GGGCCTGTTCGCCGGCTGA (SEQ ID NO: 5) |
| | MVWSVQPEAVLASAAAESAISAETEAAAAGAAPALLSTTPMGG |
| | DPDSAMFSAALNACGASYLGVVAEHASQRGLFAG |
| | (SEQ ID NO: 6) |
| Rv0285 | ATGACCCTGAGGGTGGTGCCCGAGGGCCTGGCCGCCGCCAGCG |
| (PE5) | CCGCCGTGGAGGCCCTGACCGCCAGGCTGGCCGCCGCCCACGC |
| | CAGCGCCGCCCCCGTGATCACCGCCGTGGTGCCCCCCGCCGCC |
| | GACCCCGTGAGCCTGCAGACCGCCGCCGGCTTCAGCGCCCAGG |
| | GCGTGGAGCACGCCGTGGTGACCGCCGAGGGCGTGGAGGAGCT |
| | GGGCAGGGCCGGCGTGGGCGTGGGCGAGAGCGGCGCCAGCTA |
| | CCTGGCCGGCGACGCCGCCGCCGCCGCCACCTACGGCGTGGTG |
| | GGCGGCTGA (SEQ ID NO: 7) |
| | MTLRVVPEGLAAASAAVEALTARLAAAHASAAPVITAVVPPAA |
| | DPVSLQTAAGFSAQGVEHAVVTAEGVEELGRAGVGVGESGASY |
| | LAGDAAAAATYGVVGG (SEQ ID NO: 8) |
| Rv1818c | ATGAGCTTCGTGGTGACCATCCCCGAGGCCCTGGCCGCCGTGG |
| (PE_ | CCACCGACCTGGCCGGCATCGGCAGCACCATCGGCACCGCCAA |
| PGRS33) | CGCCGCCGCCGCCGTGCCCACCACCACCGTGCTGGCCGCCGCC |
| | GCCGACGAGGTGAGCGCCGCCATGGCCGCCCTGTTCAGCGGCC |
| | ACGCCCAGGCCTACCAGGCCCTGAGCGCCCAGGCCGCCCTGTT |
| | CCACGAGCAGTTCGTGAGGGCCCTGACCGCCGGCGCCGGCAGC |
| | TACGCCGCCGCCGAGGCCGCCAGCGCCGCCCCCCTGGAGGGC |
| | TGA (SEQ ID NO: 9) |
| | MSFVVTIPEALAAVATDLAGIGSTIGTANAAAAVPTTTVLAAA |
| | ADEVSAAMAALFSGHAQAYQALSAQAALFHEQFVRALTAGAGS |
| | YAAAEAASAAPLEG (SEQ ID NO: 10) |
| Rv0159c | ATGAGCTACGTGATCGCCGCCCCCGAGATGCTGGCCACCACCG |
| (PE3) | CCGCCGACGTGGACGGCATCGGCAGCGCCATCAGGGCCGCCAG |
| | CGCCAGCGCCGCCGGCCCCACCACCGGCCTGCTGGCCGCCGCC |
| | GCCGACGAGGTGAGCAGCGCCGCCGCCGCCCTGTTCAGCGAGT |
| | ACGCCAGGGAGTGCCAGGAGGTGCTGAAGCAGGCCGCCGCCTT |
| | CCACGGCGAGTTCACCAGGGCCCTGGCCGCCGCCGGCGCCGCC |
| | TACGCCCAGGCCGAGGCCAGCAACACCGCCGCCATGAGCGGC |
| | ACCGCCGGCAGCAGCGGCGCCCTGGGCAGCTGA (SEQ ID |
| | NO: 11) |
| | MSYVIAAPEMLATTAADVDGIGSAIRAASASAAGPTTGLLAAA |
| | ADEVSSAAAALFSEYARECQEVLKQAAAFHGEFTRALAAAGAA |
| | YAQAEASNTAAMSGTAGSSGALGS (SEQ ID NO: 12) |
| Rv1172c | ATGAGCTTCGTGTTCGCCGCCCCCGAGGCCCTGGCCGCCGCCG |
| (PE12) | CCGCCGACATGGCCGGCATCGGCAGCACCCTGAACGCCGCCAA |
| | CGTGGTGGCCGCCGTGCCCACCACCGGCGTGCTGGCCGCCGCC |
| | GCCGACGAGGTGAGCACCCAGGTGGCCGCCCTGCTGAGCGCCC |
| | ACGCCCAGGGCTACCAGCAGCTGAGCAGGCAGATGATGACCGC |
| | CTTCCACGACCAGTTCGTGCAGGCCCTGAGGGCCAGCGCCGAC |
| | GCCTACGCCACCGCCGAGGCCAGCGCCGCCCAGACCATGGTGA |
| | ACGCCGTGAACGCCCCCGCCAGGGCCCTGTGA |
| | (SEQ ID NO: 13) |
| | MSFVFAAPEALAAAAADMAGIGSTLNAANVVAAVPTTGVLAAA |
| | ADEVSTQVAALLSAHAQGYQQLSRQMMTAFHDQFVQALRASAD |
| | AYATAEASAAQTMVNAVNAPARAL (SEQ ID NO: 14) |

In some embodiments, a composition comprises at least two of the PE antigens. In some embodiments, the composition comprises at least three of the PE antigens. In some embodiments, the composition comprises at least four of the PE antigens. In some embodiments, the composition comprises at least five of the PE antigens. In some embodiments, the composition comprises at least six of the PE antigens. In some embodiments, the composition comprises all seven PE antigens. In some embodiments, the composition comprises from at least two to seven of the PE antigens. In some embodiments, the composition comprises from at least three to seven of the PE antigens. In some embodiments, the composition comprises from at least four to seven of the PE antigens. In some embodiments, the composition comprises at least five to seven of the PE antigens. In some embodiments, the composition comprises six or seven of the PE antigens. In some embodiments, the composition further comprises a pharmaceutically acceptable carrier.

In some embodiments, the fusion protein comprises at least two of the PE antigens. In some embodiments, the fusion protein comprises at least three of the PE antigens. In some embodiments, the fusion protein comprises at least four of the PE antigens. In some embodiments, the fusion protein comprises at least five of the PE antigens. In some embodiments, the fusion protein comprises at least six of the PE antigens. In some embodiments, the fusion protein comprises all seven PE antigens. In some embodiments, the fusion protein comprises from at least two to seven of the PE antigens. In some embodiments, the fusion protein comprises from at least three to seven of the PE antigens. In some embodiments, the fusion protein comprises from at least four to seven of the PE antigens. In some embodiments, the fusion protein comprises at least five to seven of the PE antigens. In some embodiments, the fusion protein comprises six or seven of the PE antigens.

In some embodiments, the fusion protein comprises Rv3872 and Rv1788. In some embodiments, the fusion protein comprises Rv3893c, Rv0285, and Rv1818c. In some embodiments, the fusion protein comprises Rv0159c and Rv1172c.

In any of the embodiments of fusion proteins set forth herein, the individual PE antigens can be present in any order. For example, for a fusion protein comprising Rv3893c, Rv0285, and Rv1818c antigens, the first (or N-terminal) antigen may be Rv3893c, Rv0285, or Rv1818c; the second antigen may be Rv3893c, Rv0285, or Rv1818c (whichever one is not the first PE antigen); and the third antigen may be Rv3893c, Rv0285, or Rv1818c (whichever one is not the first or second PE antigen). Likewise for every fusion protein disclosed herein.

Individual PE antigens may be linked together in a C-terminus to N-terminus or N-terminus to C-terminus manner without any linker. Alternately, a linker may be present between any two PE antigens within any of the fusion proteins disclosed herein. In some embodiments, the linker is a segment of DNA or RNA optionally containing one or more restrictions sites, wherein the linker is inserted between nucleic acid molecules encoding two PE antigens of any of the fusion proteins disclosed herein.

In some embodiments, the fusion protein comprises Rv3872-Rv1788-Rv3893c-Rv0285-Rv1818c-Rv0159c-Rv1172c (Construct A; see Table 2). The nucleotide sequence is SEQ ID NO: 15, and the corresponding amino acid sequence is SEQ ID NO:16 (with inserted AAA between Rv1788 and Rv3893c to disrupt possible epitope with homology to human proteins; C-terminal HA tag (YPYDVPDYA; SEQ ID NO:17) added).

TABLE 2
PE Antigen Cassette

| | nucleotide sequence |
|---|---|
| Construct | amino acid sequence |
| A | ATGGAGAAGATGAGCCACGACCCCA |
| | TCGCCGCCGACATCGGCACCCAGGT |
| | GAGCGACAACGCCCTGCACGGCGTG |
| | ACCGCCGGCAGCACCGCCCTGACCA |
| | GCGTGACCGGCCTGGTGCCCGCCGG |
| | CGCCGACGAGGTGAGCGCCCAGGCC |
| | GCCACCGCCTTCACCAGCGAGGGCA |
| | TCCAGCTGCTGGCCAGCAACGCCAG |
| | CGCCCAGGACCAGCTGCACAGGGCC |
| | GGCGAGGCCGTGCAGGACGTGGCCA |
| | GGACCTACAGCCAGATCGACGACGG |
| | CGCCGCCGGCGTGTTCGCCGAGATG |
| | AGCTTCGTGACCACCCAGCCCGAGG |
| | CCCTGGCCGCCGCCGCCGGCAGCCT |
| | GCAGGGCATCGGCAGCGCCCTGAAC |
| | GCCCAGAACGCCGCCGCCGCCACCC |
| | CCACCACCGGCGTGGTGCCCGCCGC |
| | CGCCGACGAGGTGAGCGCCCTGACC |
| | GCCGCCCAGTTCGCCGCCCACGCCC |
| | AGATCTACCAGGCCGTGAGCGCCCA |
| | GGCCGCCGCCATCCACGAGATGTTC |
| | GTGAACACCCTGCAGATGAGCAGCG |
| | GCAGCTACGCCGCCACCGAGGCCGC |
| | CAACGCCGCCGCCGCCGGCGCCGCC |
| | GCCATGGTGTGGAGCGTGCAGCCCG |
| | AGGCCGTGCTGGCCAGCGCCGCCGC |
| | CGAGAGCGCCATCAGCGCCGAGACC |
| | GAGGCCGCCGCCGCCGGCGCCGCCC |
| | CCGCCCTGCTGAGCACCACCCCCAT |
| | GGGCGGCGACCCCGACAGCGCCATG |
| | TTCAGCGCCGCCCTGAACGCCTGCG |
| | GCGCCAGCTACCTGGGCGTGGTGGC |
| | CGAGCACGCCAGCCAGAGGGGCCTG |
| | TTCGCCGGCATGACCCTGAGGGTGG |
| | TGCCCGAGGGCCTGGCCGCCGCCAG |
| | CGCCGCCGTGGAGGCCCTGACCGCC |
| | AGGCTGGCCGCCGCCCACGCCAGCG |
| | CCGCCCCCGTGATCACCGCCGTGGT |
| | GCCCCCCGCCGCCGACCCCGTGAGC |
| | CTGCAGACCGCCGCCGGCTTCAGCG |
| | CCCAGGGCGTGGAGCACGCCGTGGT |
| | GACCGCCGAGGGCGTGGAGGAGCTG |
| | GGCAGGGCCGGCGTGGGCGTGGGCG |
| | AGAGCGGCGCCAGCTACCTGGCCGG |
| | CGACGCCGCCGCCGCCGCCACCTAC |
| | GGCGTGGTGGGGGGCATGAGCTTCG |
| | TGGTGACCATCCCCGAGGCCCTGGC |
| | CGCCGTGGCCACCGACCTGGCCGGC |
| | ATCGGCAGCACCATCGGCACCGCCA |
| | ACGCCGCCGCCGCCGTGCCCACCAC |
| | CACCGTGCTGGCCGCCGCCGCCGAC |
| | GAGGTGAGCGCCGCCATGGCCGCCC |
| | TGTTCAGCGGCCACGCCCAGGCCTA |
| | CCAGGCCCTGAGCGCCCAGGCCGCC |
| | CTGTTCCACGAGCAGTTCGTGAGGG |
| | CCCTGACCGCCGGCGCCGGCAGCTA |
| | CGCCGCCATGAGCTACGTGATCGCC |
| | GCCCCCGAGATGCTGGCCACCACCG |
| | CCGCCGACGTGGACGGCATCGGCAG |
| | CGCCATCAGGGCCGCCAGCGCCAGC |
| | GCCGCCGGCCCCACCACCGGCCTGC |
| | TGGCCGCCGCCGCCGACGAGGTGAG |
| | CAGCGCCGCCGCCGCCCTGTTCAGC |
| | GAGTACGCCAGGGAGTGCCAGGAGG |
| | TGCTGAAGCAGGCCGCCGCCTTCCA |
| | CGGCGAGTTCACCAGGGCCCTGGCC |
| | GCCGCCGGCGCCGCCTACGCCCAGG |
| | CCGAGGCCAGCAACACCGCCGCCAT |
| | GAGCGGCACCGCCGGCAGCAGCGGC |
| | GCCCTGGGCAGCATGAGCTTCGTGT |
| | TCGCCGCCCCCGAGGCCCTGGCCGC |
| | CGCCGCCGCCGACATGGCCGGCATC |
| | GGCAGCACCCTGAACGCCGCCAACG |
| | TGGTGGCCGCCGTGCCCACCACCGG |
| | CGTGCTGGCCGCCGCCGCCGACGAG |
| | GTGAGCACCCAGGTGGCCGCCCTGC |
| | TGAGCGCCCACGCCCAGGGCTACCA |
| | GCAGCTGAGCAGGCAGATGATGACC |
| | GCCTTCCACGACCAGTTCGTGCAGG |
| | CCCTGAGGGCCAGCGCCGACGCCTA |
| | CGCCACCGCCGAGGCCAGCGCCGCC |
| | CAGACCATGGTGAACGCCGTGAACG |
| | CCCCCGCCAGGGCCCTGTACCCCTA |
| | CGACGTGCCCGACTACGCCTGA |
| | (SEQ ID NO: 15) |
| | MEKMSHDPIAADIGTQVSDNALHGV |
| | TAGSTALTSVTGLVPAGADEVSAQA |
| | ATAFTSEGIQLLASNASAQDQLHRA |
| | GEAVQDVARTYSQIDDGAAGVFAEM |
| | SFVTTQPEALAAAAGSLQGIGSALN |
| | AQNAAAATPTTGVVPAAADEVSALT |
| | AAQFAAHAQIYQAVSAQAAAIHEMF |
| | VNTLQMSSGSYAATEAANAAAAGAA |
| | AMVWSVQPEAVLASAAAESAISAET |
| | EAAAAGAAPALLSTTPMGGDPDSAM |
| | FSAALNACGASYLGVVAEHASQRGL |
| | FAGMTLRVVPEGLAAASAAVEALTA |
| | RLAAAHASAAPVITAVVPPAADPVS |
| | LQTAAGFSAQGVEHAVVTAEGVEEL |
| | GRAGVGVGESGASYLAGDAAAAATY |
| | GVVGGMSFVVTIPEALAAVATDLAG |
| | IGSTIGTANAAAAVPTTTVLAAAAD |
| | EVSAAMAALFSGHAQAYQALSAQAA |
| | LFHEQFVRALTAGAGSYAAMSYVIA |
| | APEMLATTAADVDGIGSAIRAASAS |
| | AAGPTTGLLAAAADEVSSAAAALFS |
| | EYARECQEVLKQAAAFHGEFTRALA |
| | AAGAAYAQAEASNTAAMSGTAGSSG |
| | ALGSMSFVFAAPEALAAAAADMAGI |
| | GSTLNAANVVAAVPTTGVLAAAADE |
| | VSTQVAALLSAHAQGYQQLSRQMMT |
| | AFHDQFVQALRASADAYATAEASAA |
| | QTMVNAVNAPARALYPYDVPDYA |
| | (SEQ ID NO: 16) |

In some embodiments, the Mtb antigen is a PPE antigen. In some embodiments, the PPE antigen is Rv3873 (also known as PPE68; includes only the PPE domain), Rv1387 (also known as PPE20; includes only the PPE domain), Rv3892c (also known as PPE69; includes only the PPE domain), Rv1789 (also known as PPE26; includes only the PPE domain), Rv1800 (also known as PPE28; includes only the PPE domain), or Rv1039c (also known as PPE15; includes only the PPE domain).

A nucleotide sequence encoding Rv3873 is shown in Table 3 as SEQ ID NO:18, and an amino acid sequence of Rv3873 is shown in Table 3 as SEQ ID NO:19.

A nucleotide sequence encoding Rv1387 is shown in Table 3 as SEQ ID NO:20, and an amino acid sequence of Rv1387 is shown in Table 3 as SEQ ID NO:21.

A nucleotide sequence encoding Rv3892c is shown in Table 3 as SEQ ID NO:22, and an amino acid sequence of Rv3892c is shown in Table 3 as SEQ ID NO:23.

A nucleotide sequence encoding Rv1789 is shown in Table 3 as SEQ ID NO:24, and an amino acid sequence of Rv1789 is shown in Table 3 as SEQ ID NO:25.

A nucleotide sequence encoding Rv1800 is shown in Table 3 as SEQ ID NO:26, and an amino acid sequence of Rv1800 is shown in Table 3 as SEQ ID NO:27.

A nucleotide sequence encoding Rv1039c is shown in Table 3 as SEQ ID NO:28, and an amino acid sequence of Rv1039c is shown in Table 3 as SEQ ID NO:29.

TABLE 3
PPE Antigens

| Con- | nucleotide sequence |
|---|---|
| struct | amino acid sequence |
| Rv3873 | ATGCTGTGGCACGCCATGCCCCCCGAGCTGAACACCGCCAGGC |
| (PPE68) | TGATGGCCGGCGCCGGCCCCGCCCCCATGCTGGCCGCCGCCGC |
| | CGGCTGGCAGACCCTGAGCGCCGCCCTGGACGCCCAGGCCGTG |
| | GAGCTGACCGCCAGGCTGAACAGCCTGGGCGAGGCCTGGACCG |
| | GCGGCGGCAGCGACAAGGCCCTGGCCGCCGCCACCCCCATGGT |
| | GGTGTGGCTGCAGACCGCCAGCACCCAGGCCAAGACCAGGGCC |
| | ATGCAGGCCACCGCCCAGGCCGCCGCCTACACCCAGGCCATG |
| | GCCACCACCCCCAGCCTGCCCGAGATCGCCGCCAACCACATCA |
| | CCCAGGCCGTGCTGACCGCCACCAACTTCTTCGGCATCAACAC |
| | CATCCCCATCGCCCTGACCGAGATGGACTACTTCATCAGGATG |
| | TGGAACCAGGCCGCCCTGGCCATGGAGGTGTACCAGGCCGAGA |
| | CCGCCGTGAACACCCTGTTCGAGAAGCTGGAGCCCATGGCCAG |
| | CATCCTGGACCCCGGCGCCAGCCAGTGA |
| | (SEQ ID NO: 18) |
| | MLWHAMPPELNTARLMAGAGPAPMLAAAAGWQTLSAALDAQA |
| | VELTARLNSLGEAWTGGGSDKALAAATPMVVWLQTASTQAKTR |
| | AMQATAQAAAYTQAMATTPSLPEIAANHITQAVLTATNFFGI |
| | NTIPIALTEMDYFIRMWNQAALAMEVYQAETAVNTLFEKLEP |
| | MASILDPGASQ (SEQ ID NO: 19) |
| Rv1387 | ATGACCGAGCCCTGGATCGCCTTCCCCCCCGAGGTGCACAGCG |
| (PPE20) | CCATGCTGAACTACGGCGCCGGCGTGGGCCCCATGCTGATCAG |
| | CGCCACCCAGAACGGCGAGCTGAGCGCCCAGTACGCCGAGGCC |
| | GCCAGCGAGGTGGAGGAGCTGCTGGGCGTGGTGGCCAGCGAG |
| | GGCTGGCAGGGCCAGGCCGCCGAGGCCTTCGTGGCCGCCTACA |
| | TGCCCTTCCTGGCCTGGCTGATCCAGGCCAGCGCCGACTGCGT |
| | GGAGATGGCCGCCCAGCAGCACGTGGTGATCGAGGCCTACACC |
| | GCCGCCGTGGAGCTGATGCCCACCCAGGTGGAGCTGGCCGCCA |
| | ACCAGATCAAGCTGGCCGTGCTGGTGGCCACCAACTTCTTCGG |
| | CATCAACACCATCCCCATCGCCATCAACGAGGCCGAGTACGTG |
| | GAGATGTGGGTGAGGGCCGCCACCACCATGGCCACCTACAGCA |
| | CCGTGAGCAGGAGCGCCCTGAGCGCCATGCCCCACACCAGCCC |
| | CCCCCCCCTGATCCTGAAGAGCGACTGA |
| | (SEQ ID NO: 20) |
| | MTEPWIAFPPEVHSAMLNYGAGVGPMLISATQNGELSAQYAEA |
| | ASEVEELLGVVASEGWQGQAAEAFVAAYMPFLAWLIQASADCV |
| | EMAAQQHVVIEAYTAAVELMPTQVELAANQIKLAVLVATNFFG |
| | INTIPIAINEAEYVEMWVRAATTMATYSTVSRSALSAMPHTSP |
| | PPLILKSD (SEQ ID NO: 21) |
| Rv3892c | ATGCCCGACCCCGGCTGGGCCGCCAGGACCCCCGAGGCCAACG |
| (PPE69) | ACCTGCTGCTGACCGCCGGCACCGGCGTGGGCACCCACCTGGC |
| | CAACCAGACCGCCTGGACCACCCTGGGCGCCAGCCACCACGCC |
| | AGCGGCGTGGCCAGCGCCATCAACACCGCCGCCACCGCCGCCA |
| | GCTGGCTGGGCGTGGGCAGCGCCGCCAGCGCCCTGAACGTGAC |
| | CATGCTGAACGCCACCCTGCACGGCCTGGCCGGCTGGGTGGAC |
| | GTGAAGCCCGCCGTGGTGAGCACCGCCATCGCCGCCTTCGAG |
| | ACCGCCAACGCCGCCATGAGGCCCGCCCCCGAGTGCATGGAGA |
| | ACAGGGACGAGTGGGGCGTGGACAACGCCATCAACCCCAGCGT |
| | GCTGTGGACCCTGACCCCCAGGATCGTGAGCCTGGACGTGGAG |
| | TACTTCGGCGTGATGTGGCCCAACAACGCCGCCGTGGGCGCCA |
| | CCTACGGCGGCGTGCTGGCCGCCCTGGCCGAGAGCCTGGCCAT |
| | CCCCCCCCCCGTGGCCACCATGGGCTGA |
| | (SEQ ID NO: 22) |
| | MPDPGWAARTPEANDLLLTAGTGVGTHLANQTAWTTLGASHHA |
| | SGVASAINTAATAASWLGVGSAASALNVTMLNATLHGLAGWVD |
| | VKPAVVSTAIAAFETANAAMRPAPECMENRDEWGVDNAINPSV |
| | LWTLTPRIVSLDVEYFGVMWPNNAAVGATYGGVLAALAESLAI |
| | PPPVATMG (SEQ ID NO: 23) |
| Rv1789 | ATGGACTTCGGCGCCCTGCCCCCCGAGGTGAACAGCGTGAGGA |
| (PPE26) | TGTACGCCGGCCCCGGCAGCGCCCCCATGGTGGCCGCCGCCAG |
| | CGCCTGGAACGGCCTGGCCGCCGAGCTGAGCAGCGCCGCCACC |
| | GGCTACGAGACCGTGATCACCCAGCTGAGCAGCGAGGGCTGGC |
| | TGGGCCCCGCCAGCGCCGCCATGGCCGAGGCCGTGGCCCCCTA |
| | CGTGGCCTGGATGAGCGCCGCCGCCGCCCAGGCCGAGCAGGCC |
| | GCCACCCAGGCCAGGGCCGCCGCCGCCGCCTTCGAGGCCGCC |
| | TTCGCCGCCACCGTGCCCCCCCCCCTGATCGCCGCCAACAGGG |
| | CCAGCCTGATGCAGCTGATCAGCACCAACGTGTTCGGCCAGAA |
| | CACCAGCGCCATCGCCGCCGCCGAGGCCCAGTACGGCGAGATG |
| | TGGGCCCAGGACAGCGCCGCCATGTACGCCTACGCCGGCAGCA |
| | GCGCCAGCGCCAGCGCCGTGACCCCCTTCAGCACCCCCCCCCA |
| | GATCGCCAACCCCACCGCCCAGGGCTGA |
| | (SEQ ID NO: 24) |
| | MDFGALPPEVNSVRMYAGPGSAPMVAAASAWNGLAAELSSAAT |
| | GYETVITQLSSEGWLGPASAAMAEAVAPYVAWMSAAAAQAEQA |
| | ATQARAAAAAFEAAFAATVPPPLIAANRASLMQLISTNVFGQN |
| | TSAIAAAEAQYGEMWAQDSAAMYAYAGSSASASAVTPFSTPPQ |
| | IANPTAQG (SEQ ID NO: 25) |
| Rv1800 | ATGCTGCCCAACTTCGCCGTGCTGCCCCCCGAGGTGAACAGCG |
| (PPE28) | CCAGGGTGTTCGCCGGCGCCGGCAGCGCCCCCATGCTGGCCGC |
| | CGCCGCCGCCTGGGACGACCTGGCCAGCGAGCTGCACTGCGCC |
| | GCCATGAGCTTCGGCAGCGTGACCAGCGGCCTGGTGGTGGGCT |
| | GGTGGCAGGGCAGCGCCAGCGCCGCCATGGTGGACGCCGCCGC |
| | CAGCTACATCGGCTGGCTGAGCACCAGCGCCGCCCACGCCGAG |
| | GGCGCCGCCGGCCTGGCCAGGGCCGCCGTGAGCGTGTTCGAG |
| | GAGGCCCTGGCCGCCACCGTGCACCCCGCCATGGTGGCCGCCA |
| | ACAGGGCCCAGGTGGCCAGCCTGGTGGCCAGCAACCTGTTCGG |
| | CCAGAACGCCCCCGCCATCGCCGCCCTGGAGAGCCTGTACGAG |
| | TGCATGTGGGCCCAGGACGCCGCCGCCATGGCCGGCTACTACG |
| | TGGGCGCCAGCGCCGTGGCCACCCAGCTGGCCAGCTGGCTGCA |
| | GAGGCTGCAGAGCATCCCCGGCGCCTGA |
| | (SEQ ID NO: 26) |
| | MLPNFAVLPPEVNSARVFAGAGSAPMLAAAAAWDDLASELHCA |
| | AMSFGSVTSGLVVGWWQGSASAAMVDAAASYIGWLSTSAAHAE |
| | GAAGLARAAVSVFEEALAATVHPAMVAANRAQVASLVASNLFG |
| | QNAPAIAALESLYECMWAQDAAAMAGYYVGASAVATQLASWL |
| | QRLQSIPGA (SEQ ID NO: 27) |
| Rv1039c | ATGGACTTCGGCGCCCTGCCCCCCGAGATCAACAGCGCCAGGA |
| (PPE15) | TGTACGCCGGCGCCGGCGCCGGCCCCATGATGGCCGCCGGCGC |
| | CGCCTGGAACGGCCTGGCCGCCGAGCTGGGCACCACCGCCGCC |
| | AGCTACGAGAGCGTGATCACCAGGCTGACCACCGAGAGCTGGA |
| | TGGGCCCCGCCAGCATGGCCATGGTGGCCGCCGCCCAGCCCTA |
| | CCTGGCCTGGCTGACCTACACCGCCGAGGCCGCCGCCCACGCC |
| | GGCAGCCAGGCCATGGCCAGCGCCGCCGCCTACGAGGCCGCC |
| | TACGCCATGACCGTGCCCCCCGAGGTGGTGGCCGCCAACAGGG |
| | CCCTGCTGGCCGCCCTGGTGGCCACCAACGTGCTGGGCATCAA |
| | CACCCCCGCCATCATGGCCACCGAGGCCCTGTACGCCGAGATG |
| | TGGGCCCAGGACGCCCTGGCCATGTACGGCTACGCCGCCGCCA |
| | GCGGCGCCGCCGGCATGCTGCAGCCCCTGAGCCCCCCCAGCCA |
| | GACCACCAACCCCGGCGGCCTGGCCTGA |
| | (SEQ ID NO: 28) |
| | MDFGALPPEINSARMYAGAGAGPMMAAGAAWNGLAAELGTTAA |
| | SYESVITRLTTESWMGPASMAMVAAAQPYLAWLTYTAEAAAHA |
| | GSQAMASAAAYEAAYAMTVPPEVVAANRALLAALVATNVLGIN |
| | TPAIMATEALYAEMWAQDALAMYGYAAASGAAGMLQPLSPPSQ |
| | TTNPGGLA (SEQ ID NO: 29) |

In some embodiments, a composition comprises at least two of the PPE antigens. In some embodiments, the composition comprises at least three of the PPE antigens. In some embodiments, the composition comprises at least four of the PPE antigens. In some embodiments, the composition comprises at least five of the PPE antigens. In some embodiments, the composition comprises all six PPE antigens. In some embodiments, the composition comprises from at least two to six of the PPE antigens. In some embodiments, the composition comprises from at least three to six of the PPE antigens. In some embodiments, the composition comprises from at least four to six of the PPE antigens. In some embodiments, the composition comprises at least five or six of the PPE antigens. In some embodiments, the composition further comprises a pharmaceutically acceptable carrier.

In some embodiments, the fusion protein comprises at least two of the PPE antigens. In some embodiments, the fusion protein comprises at least three of the PPE antigens. In some embodiments, the fusion protein comprises at least four of the PPE antigens. In some embodiments, the fusion protein comprises at least five of the PPE antigens. In some embodiments, the fusion protein comprises all six PPE antigens. In some embodiments, the fusion protein comprises from at least two to six of the PPE antigens. In some embodiments, the fusion protein comprises from at least three to six of the PPE antigens. In some embodiments, the fusion protein comprises from at least four to six of the PPE antigens. In some embodiments, the fusion protein comprises at least five or six of the PPE antigens.

In some embodiments, the fusion protein comprises Rv3873, Rv1387, and Rv3892c. In some embodiments, the fusion protein comprises Rv1789, Rv1800, and Rv1039c.

In any of the embodiments of fusion proteins set forth herein, the individual PPE antigens can be present in any order. For example, for a fusion protein comprising Rv1789, Rv1800, and Rv1039c antigens, the first (or N-terminal) antigen may be Rv1789, Rv1800, or Rv1039c; the second antigen may be Rv1789, Rv1800, or Rv1039c (whichever one is not the first PPE antigen); and the third antigen may be Rv1789, Rv1800, and Rv1039c (whichever one is not the first or second PPE antigen). Likewise for every fusion protein disclosed herein.

Individual PPE antigens may be linked together in a C-terminus to N-terminus or N-terminus to C-terminus manner without any linker. Alternately, a linker may be present between any two PPE antigens within any of the fusion proteins disclosed herein. In some embodiments, the linker is a segment of DNA or RNA optionally containing one or more restrictions sites, wherein the linker is inserted between nucleic acid molecules encoding two PE antigens of any of the fusion proteins disclosed herein.

In some embodiments, the fusion protein comprises Rv3873-Rv1387-Rv3892c (Construct B; see Table 4). The nucleotide sequence is SEQ ID NO:30, and the corresponding amino acid sequence is SEQ ID NO:31 (including a C-terminal HA tag (YPYDVPDYA; SEQ ID NO:17) added).

In some embodiments, the fusion protein comprises Rv1789-Rv1800-Rv1039c (Construct C; see Table 4). The nucleotide sequence is SEQ ID NO:32, and the corresponding amino acid sequence is SEQ ID NO:33 (including a C-terminal HA tag (YPYDVPDYA; SEQ ID NO:17) added).

TABLE 4
PPE Antigen Cassette

| | nucleotide sequence |
|---|---|
| Construct | amino acid sequence |
| B | ATGCTGTGGCACGCCATGCCCCCCG |
| | AGCTGAACACCGCCAGGCTGATGGC |
| | CGGCGCCGGCCCCGCCCCCATGCTG |
| | GCCGCCGCCGCCGGCTGGCAGACCC |
| | TGAGCGCCGCCCTGGACGCCCAGGC |
| | CGTGGAGCTGACCGCCAGGCTGAAC |
| | AGCCTGGGCGAGGCCTGGACCGGCG |
| | GCGGCAGCGACAAGGCCCTGGCCGC |
| | CGCCACCCCCATGGTGGTGTGGCTG |
| | CAGACCGCCAGCACCCAGGCCAAGA |
| | CCAGGGCCATGCAGGCCACCCCCCA |
| | GGCCGCCGCCTACACCCAGGCCATG |
| | GCCACCACCCCCAGCCTGCCCGAGA |
| | TCGCCGCCAACCACATCACCCAGGC |
| | CGTGCTGACCGCCACCAACTTCTTC |
| | GGCATCAACACCATCCCCATCGCCC |
| | TGACCGAGATGGACTACTTCATCAG |
| | GATGTGGAACCAGGCCGCCCTGGCC |
| | ATGGAGGTGTACCAGGCCGAGACCG |
| | CCGTGAACACCCTGTTCGAGAAGCT |
| | GGAGCCCATGGCCAGCATCCTGGAC |
| | CCCGGCGCCAGCCAGATGACCGAGC |
| | CCTGGATCGCCTTCCCCCCCGAGGT |
| | GCACAGCGCCATGCTGAACTACGGC |
| | GCCGGCGTGGGCCCCATGCTGATCA |
| | GCGCCACCCAGAACGGCGAGCTGAG |
| | CGCCCAGTACGCCGAGGCCGCCAGC |
| | GAGGTGGAGGAGCTGCTGGGCGTGG |
| | TGGCCAGCGAGGGCTGGCAGGGCCA |
| | GGCCGCCGAGGCCTTCGTGGCCGCC |
| | TACATGCCCTTCCTGGCCTGGCTGA |
| | TCCAGGCCAGCGCCGACTGCGTGGA |
| | GATGGCCGCCCAGCAGCACGTGGTG |
| | ATCGAGGCCTACACCGCCGCCGTGG |
| | AGCTGATGCCCACCCAGGTGGAGCT |
| | GGCCGCCAACCAGATCAAGCTGGCC |
| | GTGCTGGTGGCCACCAACTTCTTCG |
| | GCATCAACACCATCCCCATCGCCAT |
| | CAACGAGGCCGAGTACGTGGAGATG |
| | TGGGTGAGGGCCGCCACCACCATGG |
| | CCACCTACAGCACCGTGAGCAGGAG |
| | CGCCCTGAGCGCCATGCCCCACACC |
| | AGCCCCCCCCCCCTGATCCTGAAGA |
| | GCGACATGCCCGACCCCGGCTGGGC |
| | CGCCAGGACCCCCGAGGCCAACGAC |
| | CTGCTGCTGACCGCCGGCACCGGCG |
| | TGGGCACCCACCTGGCCAACCAGAC |
| | CGCCTGGACCACCCTGGGCGCCAGC |
| | CACCACGCCAGCGGCGTGGCCAGCG |
| | CCATCAACACCGCCGCCACCGCCGC |
| | CAGCTGGCTGGGCGTGGGCAGCGCC |
| | GCCAGCGCCCTGAACGTGACCATGC |
| | TGAACGCCACCCTGCACGGCCTGGC |
| | CGGCTGGGTGGACGTGAAGCCCGCC |
| | GTGGTGAGCACCGCCATCGCCGCCT |
| | TCGAGACCGCCAACGCCGCCATGAG |
| | GCCCGCCCCCGAGTGCATGGAGAAC |
| | AGGGACGAGTGGGGCGTGGACAACG |
| | CCATCAACCCCAGCGTGCTGTGGAC |
| | CCTGACCCCCAGGATCGTGAGCCTG |
| | GACGTGGAGTACTTCGGCGTGATGT |
| | GGCCCAACAACGCCGCCGTGGGCGC |
| | CACCTACGGCGGCGTGCTGGCCGCC |
| | CTGGCCGAGAGCCTGGCCATCCCCC |
| | CCCCCGTGGCCACCATGGGCTACCC |
| | CTACGACGTGCCCGACTACGCCTGA |
| | (SEQ ID NO: 30) |
| | MLWHAMPPELNTARLMAGAGPAPML |
| | AAAAGWQTLSAALDAQAVELTARLN |
| | SLGEAWTGGGSDKALAAATPMVVWL |
| | QTASTQAKTRAMQATAQAAAYTQAM |
| | ATTPSLPEIAANHITQAVLTATNFF |
| | GINTIPIALTEMDYFIRMWNQAALA |
| | MEVYQAETAVNTLFEKLEPMASILD |
| | PGASQMTEPWIAFPPEVHSAMLNYG |
| | AGVGPMLISATQNGELSAQYAEAAS |
| | EVEELLGVVASEGWQGQAAEAFVAA |
| | YMPFLAWLIQASADCVEMAAQQHVV |
| | IEAYTAAVELMPTQVELAANQIKLA |
| | VLVATNFFGINTIPIAINEAEYVEM |
| | WVRAATTMATYSTVSRSALSAMPHT |
| | SPPPLILKSDMPDPGWAARTPEAND |
| | LLLTAGTGVGTHLANQTAWTTLGAS |
| | HHASGVASAINTAATAASWLGVGSA |
| | ASALNVTMLNATLHGLAGWVDVKPA |
| | VVSTAIAAFETANAAMRPAPECMEN |
| | RDEWGVDNAINPSVLWTLTPRIVSL |
| | DVEYFGVMWPNNAAVGATYGGVLAA |
| | LAESLAIPPPVATMGYPYDVPDYA |
| | (SEQ ID NO: 31) |
| C | ATGGACTTCGGCGCCCTGCCCCCCG |
| | AGGTGAACAGCGTGAGGATGTACGC |
| | CGGCCCCGGCAGCGCCCCCATGGTG |
| | GCCGCCGCCAGCGCCTGGAACGGCC |
| | TGGCCGCCGAGCTGAGCAGCGCCGC |
| | CACCGGCTACGAGACCGTGATCACC |
| | CAGCTGAGCAGCGAGGGCTGGCTGG |
| | GCCCCGCCAGCGCCGCCATGGCCGA |
| | GGCCGTGGCCCCCTACGTGGCCTGG |
| | ATGAGCGCCGCCGCCGCCCAGGCCG |
| | AGCAGGCCGCCACCCAGGCCAGGGC |
| | CGCCGCCGCCGCCTTCGAGGCCGCC |
| | TTCGCCGCCACCGTGCCCCCCCCCC |
| | TGATCGCCGCCAACAGGGCCAGCCT |
| | GATGCAGCTGATCAGCACCAACGTG |
| | TTCGGCCAGAACACCAGCGCCATCG |
| | CCGCCGCCGAGGCCCAGTACGGCGA |
| | GATGTGGGCCCAGGACAGCGCCGCC |
| | ATGTACGCCTACGCCGGCAGCAGCG |
| | CCAGCGCCAGCGCCGTGACCCCCTT |
| | CAGCACCCCCCCCCAGATCGCCAAC |
| | CCCACCGCCCAGGGCATGCTGCCCA |
| | ACTTCGCCGTGCTGCCCCCCGAGGT |
| | GAACAGCGCCAGGGTGTTCGCCGGC |
| | GCCGGCAGCGCCCCCATGCTGGCCG |
| | CCGCCGCCGCCTGGGACGACCTGGC |
| | CAGCGAGCTGCACTGCGCCGCCATG |
| | AGCTTCGGCAGCGTGACCAGCGGCC |
| | TGGTGGTGGGCTGGTGGCAGGGCAG |
| | CGCCAGCGCCGCCATGGTGGACGCC |
| | GCCGCCAGCTACATCGGCTGGCTGA |
| | GCACCAGCGCCGCCCACGCCGAGGG |
| | CGCCGCCGGCCTGGCCAGGGCCGCC |
| | GTGAGCGTGTTCGAGGAGGCCCTGG |
| | CCGCCACCGTGCACCCCGCCATGGT |
| | GGCCGCCAACAGGGCCCAGGTGGCC |
| | AGCCTGGTGGCCAGCAACCTGTTCG |
| | GCCAGAACGCCCCCGCCATCGCCGC |
| | CCTGGAGAGCCTGTACGAGTGCATG |
| | TGGGCCCAGGACGCCGCCGCCATGG |
| | CCGGCTACTACGTGGGCGCCAGCGC |
| | CGTGGCCACCCAGCTGGCCAGCTGG |
| | CTGCAGAGGCTGCAGAGCATCCCCG |
| | GCGCCATGGACTTCGGCGCCCTGCC |
| | CCCCGAGATCAACAGCGCCAGGATG |
| | TACGCCGGCGCCGGCGCCGGCCCCA |
| | TGATGGCCGCCGGCGCCGCCTGGAA |
| | CGGCCTGGCCGCCGAGCTGGGCACC |
| | ACCGCCGCCAGCTACGAGAGCGTGA |
| | TCACCAGGCTGACCACCGAGAGCTG |
| | GATGGGCCCCGCCAGCATGGCCATG |
| | GTGGCCGCCGCCCAGCCCTACCTGG |
| | CCTGGCTGACCTACACCGCCGAGGC |
| | CGCCGCCCACGCCGGCAGCCAGGCC |
| | ATGGCCAGCGCCGCCGCCTACGAGG |
| | CCGCCTACGCCATGACCGTGCCCCC |
| | CGAGGTGGTGGCCGCCAACAGGGCC |
| | CTGCTGGCCGCCCTGGTGGCCACCA |
| | ACGTGCTGGGCATCAACACCCCCGC |
| | CATCATGGCCACCGAGGCCCTGTAC |
| | GCCGAGATGTGGGCCCAGGACGCCC |
| | TGGCCATGTACGGCTACGCCGCCGC |
| | CAGCGGCGCCGCCGGCATGCTGCAG |
| | CCCCTGAGCCCCCCCAGCCAGACCA |
| | CCAACCCCGGCGGCCTGGCCTACCC |
| | CTACGACGTGCCCGACTACGCCTGA |
| | (SEQ ID NO: 32) |
| | MDFGALPPEVNSVRMYAGPGSAPMV |
| | AAASAWNGLAAELSSAATGYETVIT |
| | QLSSEGWLGPASAAMAEAVAPYVAW |
| | MSAAAAQAEQAATQARAAAAAFEAA |
| | FAATVPPPLIAANRASLMQLISTNV |
| | FGQNTSAIAAAEAQYGEMWAQDSAA |
| | MYAYAGSSASASAVTPFSTPPQIAN |
| | PTAQGMLPNFAVIPPEVNSARVFAG |
| | AGSAPMLAAAAAWDDLASELHCAAM |
| | SFGSVTSGLVVGWWQGSASAAMVDA |
| | AASYIGWLSTSAAHAEGAAGLARAA |
| | VSVFEEALAATVHPAMVAANRAQVA |
| | SLVASNLFGQNAPAIAALESLYECM |
| | WAQDAAAMAGYYVGASAVATQLASW |
| | LQRLQSIPGAMDFGALPPEINSARM |
| | YAGAGAGPMMAAGAAWNGLAAELGT |
| | TAASYESVITRLTTESWMGPASMAM |
| | VAAAQPYLAWLTYTAEAAAHAGSQA |
| | MASAAAYEAAYAMTVPPEVVAANRA |
| | LLAALVATNVLGINTPAIMATEALY |
| | AEMWAQDALAMYGYAAASGAAGMLQ |
| | PLSPPSQTTNPGGLAYPYDVPDYA |
| | (SEQ ID NO: 33) |

In some embodiments, the Mtb antigen is an ESX antigen. In some embodiments, the ESX antigen is Rv3017c (also known as esxQ), Rv3020c (also known as esxS), Rv3019c (also known as esxR), Rv3891c (also known as esxD), Rv2346c (also known as esxO), Rv3445c (also known as esxU), Rv3619c (also known as esxV), Rv3875 (also known as esxA and ESAT6), or Rv3874 (also known as esxB and CFP10).

A nucleotide sequence encoding Rv3017c is shown in Table 5 as SEQ ID NO:34, and an amino acid sequence of Rv3017c is shown in Table 5 as SEQ ID NO:35.

A nucleotide sequence encoding Rv3020c is shown in Table 5 as SEQ ID NO:36, and an amino acid sequence of Rv3020c is shown in Table 5 as SEQ ID NO:37.

A nucleotide sequence encoding Rv3019c is shown in Table 5 as SEQ ID NO:38, and an amino acid sequence of Rv3019c is shown in Table 5 as SEQ ID NO:39.

A nucleotide sequence encoding Rv3891c is shown in Table 5 as SEQ ID NO:40, and an amino acid sequence of Rv3891c is shown in Table 5 as SEQ ID NO:41.

A nucleotide sequence encoding Rv2346c is shown in Table 5 as SEQ ID NO:42, and an amino acid sequence of Rv2346c is shown in Table 5 as SEQ ID NO:43.

A nucleotide sequence encoding Rv3445c is shown in Table 5 as SEQ ID NO:44, and an amino acid sequence of Rv3445c is shown in Table 5 as SEQ ID NO:45.

A nucleotide sequence encoding Rv3619c is shown in Table 5 as SEQ ID NO:46, and an amino acid sequence of Rv3619c is shown in Table 5 as SEQ ID NO:47.

A nucleotide sequence encoding Rv3875 is shown in Table 5 as SEQ ID NO:48, and an amino acid sequence of Rv3875 is shown in Table 5 as SEQ ID NO:49.

A nucleotide sequence encoding Rv3874 is shown in Table 5 as SEQ ID NO:50, and an amino acid sequence of Rv3874 is shown in Table 5 as SEQ ID NO:51.

TABLE 5
ESX Antigens

| | nucleotide sequence |
|---|---|
| Construct | aminoacidsequence |
| Rv 3017c | GTGAGCCAGAGCATGTACAGCTACC |
| (esxQ) | CCGCCATGACCGCCAACGTGGGCGA |
| | CATGGCCGGCTACACCGGCACCACC |
| | CAGAGCCTGGGCGCCGACATCGCCA |
| | GCGAGAGGACCGCCCCCAGCAGGGC |
| | CTGCCAGGGCGACCTGGGCATGAGC |
| | CACCAGGACTGGCAGGCCCAGTGGA |
| | ACCAGGCCATGGAGGCCCTGGCCAG |
| | GGCCTACAGGAGGTGCAGGAGGGCC |
| | CTGAGGCAGATCGGCGTGCTGGAGA |
| | GGCCCGTGGGCGACAGCAGCGACTG |
| | CGGCACCATCAGGGTGGGCAGCTTC |
| | AGGGGCAGGTGGCTGGACCCCAGGC |
| | ACGCCGGCCCCGCCACCGCCGCCGA |
| | CGCCGGCGACTGA |
| | (SEQ ID NO: 34) |
| | VSQSMYSYPAMTANVGDMAGYTGTT |
| | QSLGADIASERTAPSRACQGDLGMS |
| | HQDWQAQWNQAMEALARAYRRCRRA |
| | LRQIGVLERPVGDSSDCGTIRVGSF |
| | RGRWLDPRHAGPATAADAGD |
| | (SEQ ID NO: 35) |
| Rv3020c | ATGAGCCTGCTGGACGCCCACATCC |
| (esxS) | CCCAGCTGATCGCCAGCCACACCGC |
| | CTTCGCCGCCAAGGCCGGCCTGATG |
| | AGGCACACCATCGGCCAGGCCGAGC |
| | AGCAGGCCATGAGCGCCCAGGCCTT |
| | CCACCAGGGCGAGAGCGCCGCCGCC |
| | TTCCAGGGCGCCCACGCCAGGTTCG |
| | TGGCCGCCGCCGCCAAGGTGAACAC |
| | CCTGCTGGACATCGCCCAGGCCAAC |
| | CTGGGCGAGGCCGCCGGCACCTACG |
| | TGGCCGCCGACGCCGCCGCCGCCAG |
| | CAGCTACACCGGCTTCTGA |
| | (SEQ ID NO: 36) |
| | MSLLDAHIPQLIASHTAFAAKAGLM |
| | RHTIGQAEQQAMSAQAFHQGESAAA |
| | FQGAHARFVAAAAKVNTLLDIAQAN |
| | LGEAAGTYVAADAAAASSYTGF |
| | (SEQ ID NO: 37) |
| Rv3019c | ATGAGCCAGATCATGTACAACTACC |
| (esxR) | CCGCCATGATGGCCCACGCCGGCGA |
| | CATGGCCGGCTACGCCGGCACCCTG |
| | CAGAGCCTGGGCGCCGACATCGCCA |
| | GCGAGCAGGCCGTGCTGAGCAGCGC |
| | CTGGCAGGGCGACACCGGCATCACC |
| | TACCAGGGCTGGCAGACCCAGTGGA |
| | ACCAGGCCCTGGAGGACCTGGTGAG |
| | GGCCTACCAGAGCATGAGCGGCACC |
| | CACGAGAGCAACACCATGGCCATGC |
| | TGGCCAGGGACGGCGCCGAGGCCGC |
| | CAAGTGGGGCGGCTGA |
| | (SEQ ID NO: 38) |
| | MSQIMYNYPAMMAHAGDMAGYAGTL |
| | QSLGADIASEQAVLSSAWQGDTGIT |
| | YQGWQTQWNQALEDLVRAYQSMSGT |
| | HESNTMAMLARDGAEAAKWGG |
| | (SEQ ID NO: 39) |
| Rv3891c | GTGGCCGACACCATCCAGGTGACCC |
| (esxD) | CCCAGATGCTGAGGAGCACCGCCAA |
| | CGACATCCAGGCCAACATGGAGCAG |
| | GCCATGGGCATCGCCAAGGGCTACC |
| | TGGCCAACCAGGAGAACGTGATGAA |
| | CCCCGCCACCTGGAGCGGCACCGGC |
| | GTGGTGGCCAGCCACATGACCGCCA |
| | CCGAGATCACCAACGAGCTGAACAA |
| | GGTGCTGACCGGCGGCACCAGGCTG |
| | GCCGAGGGCCTGGTGCAGGCCGCCG |
| | CCCTGATGGAGGGCCACGAGGCCGA |
| | CAGCCAGACCGCCTTCCAGGCCCTG |
| | TTCGGCGCCAGCCACGGCAGCTGA |
| | (SEQ ID NO: 40) |
| | VADTIQVTPQMLRSTANDIQANMEQ |
| | AMGIAKGYLANQENVMNPATWSGTG |
| | VVASHMTATEITNELNKVLTGGTRL |
| | AEGLVQAAALMEGHEADSQTAFQAL |
| | FGASHGS |
| | (SEQ ID NO: 41) |
| Rv2346c | ATGACCATCAACTACCAGTTCGGCG |
| (esxO) | ACGTGGACGCCCACGGCGCCATGAT |
| | CAGGGCCCAGGCCGGCCTGCTGGAG |
| | GCCGAGCACCAGGCCATCGTGAGGG |
| | ACGTGCTGGCCGCCGGCGACTTCTG |
| | GGGCGGCGCCGGCAGCGTGGCCTGC |
| | CAGGAGTTCATCACCCAGCTGGGCA |
| | GGAACTTCCAGGTGATCTACGAGCA |
| | GGCCAACGCCCACGGCCAGAAGGTG |
| | CAGGCCGCCGGCAACAACATGGCCC |
| | AGACCGACAGCGCCGTGGGCAGCAG |
| | CTGGGCCTGA |
| | (SEQ ID NO: 42) |
| | MTINYQFGDVDAHGAMIRAQAGLLE |
| | AEHQAIVRDVLAAGDFWGGAGSVAC |
| | QEFITQLGRNFQVIYEQANAHGQKV |
| | QAAGNNMAQTDSAVGSSWA |
| | (SEQ ID NO: 43) |
| Rv3445c | GTGAGCACCCCCAACACCCTGAACG |
| (esxU) | CCGACTTCGACCTGATGAGGAGCGT |
| | GGCCGGCATCACCGACGCCAGGAAC |
| | GAGGAGATCAGGGCCATGCTGCAGG |
| | CCTTCATCGGCAGGATGAGCGGCGT |
| | GCCCCCCAGCGTGTGGGGCGGCCTG |
| | GCCGCCGCCAGGTTCCAGGACGTGG |
| | TGGACAGGTGGAACGCCGAGAGCAC |
| | CAGGCTGTACCACGTGCTGCACGCC |
| | ATCGCCGACACCATCAGGCACAACG |
| | AGGCCGCCCTGAGGGAGGCCGGCCA |
| | GATCCACGCCAGGCACATCGCCGCC |
| | GCCGGCGGCGACCTGTGA |
| | (SEQ ID NO: 44) |
| | VSTPNTLNADFDLMRSVAGITDARN |
| | EEIRAMLQAFIGRMSGVPPSVWGGL |
| | AAARFQDVVDRWNAESTRLYHVLHA |
| | IADTIRHNEAALREAGQIHARHIAA |
| | AGGDL |
| | (SEQ ID NO: 45) |
| Rv3619c | ATGACCATCAACTACCAGTTCGGCG |
| (esx V) | ACGTGGACGCCCACGGCGCCATGAT |
| | CAGGGCCCAGGCCGGCAGCCTGGAG |
| | GCCGAGCACCAGGCCATCATCAGCG |
| | ACGTGCTGACCGCCAGCGACTTCTG |
| | GGGCGGCGCCGGCAGCGCCGCCTGC |
| | CAGGGCTTCATCACCCAGCTGGGCA |
| | GGAACTTCCAGGTGATCTACGAGCA |
| | GGCCAACGCCCACGGCCAGAAGGTG |
| | CAGGCCGCCGGCAACAACATGGCCC |
| | AGACCGACAGCGCCGTGGGCAGCAG |
| | CTGGGCCTGA |
| | (SEQ ID NO: 46) |
| | MTINYQFGDVDAHGAMIRAQAGSLE |
| | AEHQAIISDVLTASDFWGGAGSAAC |
| | QGFITQLGRNFQVIYEQANAHGQKV |
| | QAAGNNMAQTDSAVGSSWA |
| | (SEQ ID NO: 47) |
| Rv3875 | ATGACCGAGCAGCAGTGGAACTTCG |
| (esxA, | CCGGCATCGAGGCCGCCGCCAGCGC |
| ESAT6) | CATCCAGGGCAACGTGACCAGCATC |
| | CACAGCCTGCTGGACGAGGGCAAGC |
| | AGAGCCTGACCAAGCTGGCCGCCGC |
| | CTGGGGCGGCAGCGGCAGCGAGGCC |
| | TACCAGGGCGTGCAGCAGAAGTGGG |
| | ACGCCACCGCCACCGAGCTGAACAA |
| | CGCCCTGCAGAACCTGGCCAGGACC |
| | ATCAGCGAGGCCGGCCAGGCCATGG |
| | CCAGCACCGAGGGCAACGTGACCGG |
| | CATGTTCGCCTGA |
| | (SEQ ID NO: 48) |
| | MTEQQWNFAGIEAAASAIQGNVTSI |
| | HSLLDEGKQSLTKLAAAWGGSGSEA |
| | YQGVQQKWDATATELNNALQNLART |
| | ISEAGQAMASTEGNVTGMFA |
| | (SEQ ID NO: 49) |
| Rv3874 | ATGGCCGAGATGAAGACCGACGCCG |
| (esxB, | CCACCCTGGCCCAGGAGGCCGGCAA |
| CFP10) | CTTCGAGAGGATCAGCGGCGACCTG |
| | AAGACCCAGATCGACCAGGTGGAGA |
| | GCACCGCCGGCAGCCTGCAGGGCCA |
| | GTGGAGGGGCGCCGCCGGCACCGCC |
| | GCCCAGGCCGCCGTGGTGAGGTTCC |
| | AGGAGGCCGCCAACAAGCAGAAGCA |
| | GGAGCTGGACGAGATCAGCACCAAC |
| | ATCAGGCAGGCCGGCGTGCAGTACA |
| | GCAGGGCCGACGAGGAGCAGCAGCA |
| | GGCCCTGAGCAGCCAGATGGGCTTC |
| | TGA |
| | (SEQ ID NO: 50) |
| | MAEMKTDAATLAQEAGNFERISGDL |
| | KTQIDQVESTAGSLQGQWRGAAGTA |
| | AQAAVVRFQEAANKOKQELDEISTN |
| | IRQAGVQYSRADEEQQQALSSQMGF |
| | (SEQ ID NO: 51) |

In some embodiments, a composition comprises at least two of the ESX antigens. In some embodiments, the composition comprises at least three of the ESX antigens. In some embodiments, the composition comprises at least four of the ESX antigens. In some embodiments, the composition comprises at least five of the ESX antigens. In some embodiments, the composition comprises at least six of the ESX antigens. In some embodiments, the composition comprises at least seven of the ESX antigens. In some embodiments, the composition comprises at least eight of the ESX antigens. In some embodiments, the composition comprises all nine ESX antigens. In some embodiments, the composition comprises from at least two to nine of the ESX antigens. In some embodiments, the composition comprises from at least three to nine of the ESX antigens. In some embodiments, the composition comprises from at least four to nine of the ESX antigens. In some embodiments, the composition comprises at least five to nine of the ESX antigens. In some embodiments, the composition comprises at least six to nine of the ESX antigens. In some embodiments, the composition comprises at least seven to nine of the ESX antigens. In some embodiments, the composition comprises eight or nine of the ESX antigens. In some embodiments, the composition further comprises a pharmaceutically acceptable carrier.

In some embodiments, the fusion protein comprises at least two of the ESX antigens. In some embodiments, the fusion protein comprises at least three of the ESX antigens. In some embodiments, the fusion protein comprises at least four of the ESX antigens. In some embodiments, the fusion protein comprises at least five of the ESX antigens. In some embodiments, the fusion protein comprises at least six of the ESX antigens. In some embodiments, the fusion protein comprises at least seven of the ESX antigens. In some embodiments, the fusion protein comprises at least eight of the ESX antigens. In some embodiments, the fusion protein comprises all nine ESX antigens. In some embodiments, the fusion protein comprises from at least two to nine of the ESX antigens. In some embodiments, the fusion protein comprises from at least three to nine of the ESX antigens. In some embodiments, the fusion protein comprises from at least four to nine of the ESX antigens. In some embodiments, the fusion protein comprises at least five to nine of the ESX antigens. In some embodiments, the fusion protein comprises at least six to nine of the ESX antigens. In some embodiments, the fusion protein comprises at least seven to nine of the ESX antigens. In some embodiments, the fusion protein comprises eight or nine of the ESX antigens.

In some embodiments, the fusion protein comprises Rv3017c, Rv3020c, Rv3019c, Rv3891c, Rv2346c, and Rv3445c. In some embodiments, the fusion protein comprises Rv3619c,

Rv3875, and Rv3874.

In any of the embodiments of fusion proteins set forth herein, the individual ESX antigens can be present in any order. For example, for a fusion protein comprising Rv3619c, Rv3875, and Rv3874 antigens, the first (or N-terminal) antigen may be Rv3619c, Rv3875, or Rv3874; the second antigen may be Rv3619c, Rv3875, or Rv3874 (whichever one is not the first ESX antigen); and the third antigen may be Rv3619c, Rv3875, or Rv3874 (whichever one is not the first or second ESX antigen). Likewise for every fusion protein disclosed herein.

Individual ESX antigens may be linked together in a C-terminus to N-terminus or N-terminus to C-terminus manner without any linker. Alternately, a linker may be present between any two ESX antigens within any of the fusion proteins disclosed herein. In some embodiments, the linker is a segment of DNA or RNA optionally containing one or more restrictions sites, wherein the linker is inserted between nucleic acid molecules encoding two ESX antigens of any of the fusion proteins disclosed herein.

In some embodiments, the fusion protein comprises Rv3017c-Rv3020c-Rv3019c-Rv3891c-Rv2346c-Rv3445c (Construct D; see Table 6). The nucleotide sequence is SEQ ID NO:52, and the corresponding amino acid sequence is SEQ ID NO:53 (including a C-terminal HA tag (YPYDVPDYA; SEQ ID NO:17) added).

In some embodiments, the fusion protein comprises Rv3619c-Rv3875-Rv3874 (Construct E; see Table 6). The nucleotide sequence is SEQ ID NO:54, and the corresponding amino acid sequence is SEQ ID NO:55 (including a C-terminal HA tag (YPYDVPDYA; SEQ ID NO:17) added).

TABLE 6
ESX Antigen Cassette

| | nucleotide sequence |
|---|---|
| Construct | amino acid sequence |
| D | GTGAGCCAGAGCATGTACAGCTACC |
| | CCGCCATGACCGCCAACGTGGGCGA |
| | CATGGCCGGCTACACCGGCACCACC |
| | CAGAGCCTGGGCGCCGACATCGCCA |
| | GCGAGAGGACCGCCCCCAGCAGGGC |
| | CTGCCAGGGCGACCTGGGCATGAGC |
| | CACCAGGACTGGCAGGCCCAGTGGA |
| | ACCAGGCCATGGAGGCCCTGGCCAG |
| | GGCCTACAGGAGGTGCAGGAGGGCC |
| | CTGAGGCAGATCGGCGTGCTGGAGA |
| | GGCCCGTGGGCGACAGCAGCGACTG |
| | CGGCACCATCAGGGTGGGCAGCTTC |
| | AGGGGCAGGTGGCTGGACCCCAGGC |
| | ACGCCGGCCCCGCCACCGCCGCCGA |
| | CGCCGGCGACATGAGCCTGCTGGAC |
| | GCCCACATCCCCCAGCTGATCGCCA |
| | GCCACACCGCCTTCGCCGCCAAGGC |
| | CGGCCTGATGAGGCACACCATCGGC |
| | CAGGCCGAGCAGCAGGCCATGAGCG |
| | CCCAGGCCTTCCACCAGGGCGAGAG |
| | CGCCGCCGCCTTCCAGGGCGCCCAC |
| | GCCAGGTTCGTGGCCGCCGCCGCCA |
| | AGGTGAACACCCTGCTGGACATCGC |
| | CCAGGCCAACCTGGGCGAGGCCGCC |
| | GGCACCTACGTGGCCGCCGACGCCG |
| | CCGCCGCCAGCAGCTACACCGGCTT |
| | CATGAGCCAGATCATGTACAACTAC |
| | CCCGCCATGATGGCCCACGCCGGCG |
| | ACATGGCCGGCTACGCCGGCACCCT |
| | GCAGAGCCTGGGCGCCGACATCGCC |
| | AGCGAGCAGGCCGTGCTGAGCAGCG |
| | CCTGGCAGGGCGACACCGGCATCAC |
| | CTACCAGGGCTGGCAGACCCAGTGG |
| | AACCAGGCCCTGGAGGACCTGGTGA |
| | GGGCCTACCAGAGCATGAGCGGCAC |
| | CCACGAGAGCAACACCATGGCCATG |
| | CTGGCCAGGGACGGCGCCGAGGCCG |
| | CCAAGTGGGGCGGCGTGGCCGACAC |
| | CATCCAGGTGACCCCCCAGATGCTG |
| | AGGAGCACCGCCAACGACATCCAGG |
| | CCAACATGGAGCAGGCCATGGGCAT |
| | CGCCAAGGGCTACCTGGCCAACCAG |
| | GAGAACGTGATGAACCCCGCCACCT |
| | GGAGCGGCACCGGCGTGGTGGCCAG |
| | CCACATGACCGCCACCGAGATCACC |
| | AACGAGCTGAACAAGGTGCTGACCG |
| | GCGGCACCAGGCTGGCCGAGGGCCT |
| | GGTGCAGGCCGCCGCCCTGATGGAG |
| | GGCCACGAGGCCGACAGCCAGACCG |
| | CCTTCCAGGCCCTGTTCGGCGCCAG |
| | CCACGGCAGCATGACCATCAACTAC |
| | CAGTTCGGCGACGTGGACGCCCACG |
| | GCGCCATGATCAGGGCCCAGGCCGG |
| | CCTGCTGGAGGCCGAGCACCAGGCC |
| | ATCGTGAGGGACGTGCTGGCCGCCG |
| | GCGACTTCTGGGGCGGCGCCGGCAG |
| | CGTGGCCTGCCAGGAGTTCATCACC |
| | CAGCTGGGCAGGAACTTCCAGGTGA |
| | TCTACGAGCAGGCCAACGCCCACGG |
| | CCAGAAGGTGCAGGCCGCCGGCAAC |
| | AACATGGCCCAGACCGACAGCGCCG |
| | TGGGCAGCAGCTGGGCCGTGAGCAC |
| | CCCCAACACCCTGAACGCCGACTTC |
| | GACCTGATGAGGAGCGTGGCCGGCA |
| | TCACCGACGCCAGGAACGAGGAGAT |
| | CAGGGCCATGCTGCAGGCCTTCATC |
| | GGCAGGATGAGCGGCGTGCCCCCCA |
| | GCGTGTGGGGCGGCCTGGCCGCCGC |
| | CAGGTTCCAGGACGTGGTGGACAGG |
| | TGGAACGCCGAGAGCACCAGGCTGT |
| | ACCACGTGCTGCACGCCATCGCCGA |
| | CACCATCAGGCACAACGAGGCCGCC |
| | CTGAGGGAGGCCGGCCAGATCCACG |
| | CCAGGCACATCGCCGCCGCCGGCGG |
| | CGACCTGTACCCCTACGACGTGCCC |
| | GACTACGCCTGA |
| | (SEQ ID NO: 52) |
| | VSQSMYSYPAMTANVGDMAGYTGTT |
| | QSLGADIASERTAPSRACQGDLGMS |
| | HQDWQAQWNQAMEALARAYRRCRRA |
| | LRQIGVLERPVGDSSDCGTIRVGSF |
| | RGRWLDPRHAGPATAADAGDMSLLD |
| | AHIPQLIASHTAFAAKAGLMRHTIG |
| | QAEQQAMSAQAFHQGESAAAFQGAH |
| | ARFVAAAAKVNTLLDIAQANLGEAA |
| | GTYVAADAAAASSYTGFMSQIMYNY |
| | PAMMAHAGDMAGYAGTLQSLGADIA |
| | SEQAVLSSAWQGDTGITYQGWQTQW |
| | NQALEDLVRAYQSMSGTHESNTMAM |
| | LARDGAEAAKWGGVADTIQVTPQML |
| | RSTANDIQANMEQAMGIAKGYLANQ |
| | ENVMNPATWSGTGVVASHMTATEIT |
| | NELNKVLTGGTRLAEGLVQAAALME |
| | GHEADSQTAFQALFGASHGSMTINY |
| | QFGDVDAHGAMIRAQAGLLEAEHQA |
| | IVRDVLAAGDFWGGAGSVACQEFIT |
| | QLGRNFQVIYEQANAHGQKVQAAGN |
| | NMAQTDSAVGSSWAVSTPNTLNADF |
| | DLMRSVAGITDARNEEIRAMLQAFI |
| | GRMSGVPPSVWGGLAAARFQDVVDR |
| | WNAESTRLYHVLHAIADTIRHNEAA |
| | LREAGQIHARHIAAAGGDLYPYDVP |
| | DYA |
| | (SEQ ID NO: 53) |
| E | ATGACCATCAACTACCAGTTCGGCG |
| | ACGTGGACGCCCACGGCGCCATGAT |
| | CAGGGCCCAGGCCGGCAGCCTGGAG |
| | GCCGAGCACCAGGCCATCATCAGCG |
| | ACGTGCTGACCGCCAGCGACTTCTG |
| | GGGCGGCGCCGGCAGCGCCGCCTGC |
| | CAGGGCTTCATCACCCAGCTGGGCA |
| | GGAACTTCCAGGTGATCTACGAGCA |
| | GGCCAACGCCCACGGCCAGAAGGTG |
| | CAGGCCGCCGGCAACAACATGGCCC |
| | AGACCGACAGCGCCGTGGGCAGCAG |
| | CTGGGCCATGACCGAGCAGCAGTGG |
| | AACTTCGCCGGCATCGAGGCCGCCG |
| | CCAGCGCCATCCAGGGCAACGTGAC |
| | CAGCATCCACAGCCTGCTGGACGAG |
| | GGCAAGCAGAGCCTGACCAAGCTGG |
| | CCGCCGCCTGGGGCGGCAGCGGCAG |
| | CGAGGCCTACCAGGGCGTGCAGCAG |
| | AAGTGGGACGCCACCGCCACCGAGC |
| | TGAACAACGCCCTGCAGAACCTGGC |
| | CAGGACCATCAGCGAGGCCGGCCAG |
| | GCCATGGCCAGCACCGAGGGCAACG |
| | TGACCGGCATGTTCGCCATGGCCGA |
| | GATGAAGACCGACGCCGCCACCCTG |
| | GCCCAGGAGGCCGGCAACTTCGAGA |
| | GGATCAGCGGCGACCTGAAGACCCA |
| | GATCGACCAGGTGGAGAGCACCGCC |
| | GGCAGCCTGCAGGGCCAGTGGAGGG |
| | GCGCCGCCGGCACCGCCGCCCAGGC |
| | CGCCGTGGTGAGGTTCCAGGAGGCC |
| | GCCAACAAGCAGAAGCAGGAGCTGG |
| | ACGAGATCAGCACCAACATCAGGCA |
| | GGCCGGCGTGCAGTACAGCAGGGCC |
| | GACGAGGAGCAGCAGCAGGCCCTGA |
| | GCAGCCAGATGGGCTTCTACCCCTA |
| | CGACGTGCCCGACTACGCCTGA |
| | (SEQ ID NO: 54) |
| | MTINYQFGDVDAHGAMIRAQAGSLE |
| | AEHQAIISDVLTASDFWGGAGSAAC |
| | QGFITQLGRNFQVIYEQANAHGQKV |
| | QAAGNNMAQTDSAVGSSWAMTEQQW |
| | NFAGIEAAASAIQGNVTSIHSLLDE |
| | GKQSLTKLAAAWGGSGSEAYQGVQQ |
| | KWDATATELNNALQNLARTISEAGQ |
| | AMASTEGNVTGMFAMAEMKTDAATL |
| | AQEAGNFERISGDLKTQIDQVESTA |
| | GSLQGQWRGAAGTAAQAAVVRFQEA |
| | ANKQKQELDEISTNIRQAGVQYSRA |
| | DEEQQQALSSQMGFYPYDVPDYA |
| | (SEQ ID NO: 55) |

In some embodiments, the Mtb antigen is a variable antigen. In some embodiments, the variable antigen is Rv2719c, Rv0010c, Rv1872c, Rv0012, Rv0990c, or Rv0995.

A nucleotide sequence encoding Rv2719c is shown in Table 7 as SEQ ID NO:56, and an amino acid sequence of Rv2719c is shown in Table 7 as SEQ ID NO:57.

A nucleotide sequence encoding Rv0010c is shown in Table 7 as SEQ ID NO:58, and an amino acid sequence of Rv0010c is shown in Table 7 as SEQ ID NO:59.

A nucleotide sequence encoding Rv1872c is shown in Table 7 as SEQ ID NO:60, and an amino acid sequence of Rv1872c is shown in Table 7 as SEQ ID NO:61.

A nucleotide sequence encoding Rv0012 is shown in Table 7 as SEQ ID NO:62, and an amino acid sequence of Rv0012 is shown in Table 7 as SEQ ID NO:63.

A nucleotide sequence encoding Rv0990c is shown in Table 7 as SEQ ID NO:64, and an amino acid sequence of Rv0990c is shown in Table 7 as SEQ ID NO:65.

A nucleotide sequence encoding Rv0995 is shown in Table 7 as SEQ ID NO:66, and an amino acid sequence of Rv0995 is shown in Table 7 as SEQ ID NO:67.

TABLE 7
Variable Antigens

| | Nucleotide sequence |
|---|---|
| Construct | amino acid sequence |
| Rv2719c | ATGACCCCCGTGAGGCCCCCCCACA |
| | CCCCCGACCCCCTGAACCTGAGGGG |
| | CCCCCTGGACGGCCCCAGGTGGAGG |
| | AGGGCCGAGCCCGCCCAGAGCAGGA |
| | GGCCCGGCAGGAGCAGGCCCGGCGG |
| | CGCCCCCCTGAGGTACCACAGGACC |
| | GGCGTGGGCATGAGCAGGACCGGCC |
| | ACGGCAGCAGGCCCGTGCCCCCCGC |
| | CACCACCGTGGGCCTGGCCCTGCTG |
| | GCCGCCGCCATCACCCTGTGGCTGG |
| | GCCTGGTGGCCCAGTTCGGCCAGAT |
| | GATCACCGGCGGCAGCGCCGACGGC |
| | AGCGCCGACAGCACCGGCAGGGTGC |
| | CCGACAGGCTGGCCGTGGTGAGGGT |
| | GGAGACCGGCGAGAGCCTGTACGAC |
| | GTGGCCGTGAGGGTGGCCCCCAACG |
| | CCCCCACCAGGCAGGTGGCCGACAG |
| | GATCAGGGAGCTGAACGGCCTGCAG |
| | ACCCCCGCCCTGGCCGTGGGCCAGA |
| | CCCTGATCGCCCCCGTGGGCTGA |
| | (SEQ ID NO: 56) |
| | MTPVRPPHTPDPLNLRGPLDGPRWR |
| | RAEPAQSRRPGRSRPGGAPLRYHRT |
| | GVGMSRTGHGSRPVPPATTVGLALL |
| | AAAITLWLGLVAQFGQMITGGSADG |
| | SADSTGRVPDRLAVVRVETGESLYD |
| | VAVRVAPNAPTROVADRIRELNGLQ |
| | TPALAVGOTLIAPVG |
| | (SEQ ID NO: 57) |
| Rv0010c | ATGCAGCAGACCGCCTGGGCCCCCA |
| | GGACCAGCGGCATCGCCGGCTGCGG |
| | CGCCGGCGGCGTGGTGATGGCCATC |
| | GCCAGCGTGACCCTGGTGACCGACA |
| | CCCCCGGCAGGGTGCTGACCGGCGT |
| | GGCCGCCCTGGGCCTGATCCTGTTC |
| | GCCAGCGCCACCTGGAGGGCCAGGC |
| | CCAGGCTGGCCATCACCCCCGACGG |
| | CCTGGCCATCAGGGGCTGGTTCAGG |
| | ACCCAGCTGCTGAGGCACAGCAACA |
| | TCAAGATCATCAGGATCGACGAGTT |
| | CAGGAGGTACGGCAGGCTGGTGAGG |
| | CTGCTGGAGATCGAGACCGTGAGCG |
| | GCGGCCTGCTGATCCTGAGCAGGTG |
| | GGACCTGGGCACCGACCCCGTGGAG |
| | GTGCTGGACGCCCTGACCGCCGCCG |
| | GCTACGCCGGCAGGGGCCAGAGGTG |
| | A |
| | (SEQ ID NO: 58) |
| | MQQTAWAPRTSGIAGCGAGGVVMAI |
| | ASVTLVTDTPGRVLTGVAALGLILF |
| | ASATWRARPRLAITPDGLAIRGWFR |
| | TQLLRHSNIKIIRIDEFRRYGRLVR |
| | LLEIETVSGGLLILSRWDLGTDPVE |
| | VLDALTAAGYAGRGQR |
| | (SEQ ID NO: 59) |
| Rv1872c | ATGGCCGTGAACAGGAGGGTGCCCA |
| | GGGTGAGGGACCTGGCCCCCCTGCT |
| | GCAGTTCAACAGGCCCCAGTTCGAC |
| | ACCAGCAAGAGGAGGCTGGGCGCCG |
| | CCCTGACCATCCAGGACCTGAGGAG |
| | GATCGCCAAGAGGAGGACCCCCAGG |
| | GCCGCCTTCGACTACGCCGACGGCG |
| | GCGCCGAGGACGAGCTGAGCATCGC |
| | CAGGGCCAGGCAGGGCTTCAGGGAC |
| | ATCGAGTTCCACCCCACCATCCTGA |
| | GGGACGTGACCACCGTGTGCGCCGG |
| | CTGGAACGTGCTGGGCCAGCCCACC |
| | GTGCTGCCCTTCGGCATCGCCCCCA |
| | CCGGCTTCACCAGGCTGATGCACAC |
| | CGAGGGCGAGATCGCCGGCGCCAGG |
| | GCCGCCGCCGCCGCCGGCATCCCCT |
| | TCAGCCTGAGCACCCTGGCCACCTG |
| | CGCCATCGAGGACCTGGTGATCGCC |
| | GTGCCCCAGGGCAGGAAGTGGTTCC |
| | AGCTGTACATGTGGAGGGACAGGGA |
| | CAGGAGCATGGCCCTGGTGAGGAGG |
| | GTGGCCGCCGCCGGCTTCGACACCA |
| | TGCTGGTGACCGTGGACGTGCCCGT |
| | GGCCGGCGCCAGGCTGAGGGACGTG |
| | AGGAACGGCATGAGCATCCCCCCCG |
| | CCCTGACCCTGAGGACCGTGCTGGA |
| | CGCCATGGGCCACCCCAGGTGGTGG |
| | TTCGACCTGCTGACCACCGAGCCCC |
| | TGGCCTTCGCCAGCCTGGACAGGTG |
| | GCCCGGCACCGTGGGCGAGTACCTG |
| | AACACCGTGTTCGACCCCAGCCTGA |
| | CCTTCGACGACCTGGCCTGGATCAA |
| | GAGCCAGTGGCCCGGCAAGCTGGTG |
| | GTGAAGGGCATCCAGACCCTGGACG |
| | ACGCCAGGGCCGTGGTGGACAGGGG |
| | CGTGGACGGCATCGTGCTGAGCAAC |
| | CACGGCGGCAGGCAGCTGGACAGGG |
| | CCCCCGTGCCCTTCCACCTGCTGCC |
| | CCACGTGGCCAGGGAGCTGGGCAAG |
| | CACACCGAGATCCTGGTGGACACCG |
| | GCATCATGAGCGGCGCCGACATCGT |
| | GGCCGCCATCGCCCTGGGCGCCAGG |
| | TGCACCCTGATCGGCAGGGCCTACC |
| | TGTACGGCCTGATGGCCGGCGGCGA |
| | GGCCGGCGTGAACAGGGCCATCGAG |
| | ATCCTGCAGACCGGCGTGATCAGGA |
| | CCATGAGGCTGCTGGGCGTGACCTG |
| | CCTGGAGGAGCTGAGCCCCAGGCAC |
| | GTGACCCAGCTGAGGAGGCTGGGCC |
| | CCATCGGCGCCCCCACCTGA |
| | (SEQ ID NO: 60) |
| | MAVNRRVPRVRDLAPLLQFNRPQFD |
| | TSKRRLGAALTIQDLRRIAKRRTPR |
| | AAFDYADGGAEDELSIARARQGFRD |
| | IEFHPTILRDVTTVCAGWNVLGQPT |
| | VLPFGIAPTGFTRLMHTEGEIAGAR |
| | AAAAAGIPFSLSTLATCAIEDLVIA |
| | VPQGRKWFQLYMWRDRDRSMALVRR |
| | VAAAGFDTMLVTVDVPVAGARLRDV |
| | RNGMSIPPALTLRTVLDAMGHPRWW |
| | FDLLTTEPLAFASLDRWPGTVGEYL |
| | NTVFDPSLTFDDLAWIKSQWPGKLV |
| | VKGIQTLDDARAVVDRGVDGIVLSN |
| | HGGRQLDRAPVPFHLLPHVARELGK |
| | HTEILVDTGIMSGADIVAAIALGAR |
| | CTLIGRAYLYGLMAGGEAGVNRAIE |
| | ILQTGVIRTMRLLGVTCLEELSPRH |
| | VTQLRRLGPIGAPT |
| | (SEQ ID NO: 61) |
| Rv0012 | ATGAGGCTGACCCACCCCACCCCCT |
| | GCCCCGAGAACGGCGAGACCATGAT |
| | CGACAGGAGGAGGAGCGCCTGGAGG |
| | TTCAGCGTGCCCCTGGTGTGCCTGC |
| | TGGCCGGCCTGCTGCTGGCCGCCAC |
| | CCACGGCGTGAGCGGCGGCACCGAG |
| | ATCAGGAGGAGCGACGCCCCCAGGC |
| | TGGTGGACCTGGTGAGGAGGGCCCA |
| | GGCCAGCGTGAACAGGCTGGCCACC |
| | GAGAGGGAGGCCCTGACCACCAGGA |
| | TCGACAGCGTGCACGGCAGGAGCGT |
| | GGACACCGCCCTGGCCGCCATGCAG |
| | AGGAGGAGCGCCAAGCTGGCCGGCG |
| | TGGCCGCCATGAACCCCGTGCACGG |
| | CCCCGGCCTGGTGGTGACCCTGCAG |
| | GACGCCCAGAGGGACGCCAACGGCA |
| | GGTTCCCCAGGGACGCCAGCCCCGA |
| | CGACCTGGTGGTGCACCAGCAGGAC |
| | ATCGAGGCCGTGCTGAACGCCCTGT |
| | GGAACGCCGGCGCCGAGGCCATCCA |
| | GATGCAGGACCAGAGGATCATCGCC |
| | ATGAGCATCGCCAGGTGCGTGGGCA |
| | ACACCCTGCTGCTGAACGGCAGGAC |
| | CTACAGCCCCCCCTACACCATCGCC |
| | GCCATCGGCGACGCCGCCGCCATGC |
| | AGGCCGCCCTGGCCGCCGCCCCCCT |
| | GGTGACCCTGTACAAGCAGTACGTG |
| | GTGAGGTTCGGCCTGGGCTACTGCG |
| | AGGAGGTGCACCCCGACCTGCAGAT |
| | CGTGGGCTACGCCGACCCCGTGAGG |
| | ATGCACTTCGCCCAGCCCGCCGGCC |
| | CCCTGGACTACTGA |
| | (SEQ ID NO: 62) |
| | MRLTHPTPCPENGETMIDRRRSAWR |
| | FSVPLVCLLAGLLLAATHGVSGGTE |
| | IRRSDAPRLVDLVRRAQASVNRLAT |
| | EREALTTRIDSVHGRSVDTALAAMQ |
| | RRSAKLAGVAAMNPVHGPGLVVTLQ |
| | DAQRDANGRFPRDASPDDLVVHQQD |
| | IEAVLNALWNAGAEAIQMQDQRIIA |
| | MSIARCVGNTLLLNGRTYSPPYTIA |
| | AIGDAAAMQAALAAAPLVTLYKQYV |
| | VRFGLGYCEEVHPDLQIVGYADPVR |
| | MHFAQPAGPLDY |
| | (SEQ ID NO: 63) |
| Rv0990c | GTGGCCGAGAGCAGCCTGAACCCCA |
| | GCCTGGTGAGCAGGATCAGCGCCTT |
| | CCTGAGGCCCGACTGGACCAGGACC |
| | GTGAGGGCCAGGAGGTTCGCCGCCG |
| | CCGGCCTGGTGATGCTGGCCGGCGT |
| | GGCCGCCCTGAGGAGCAACCCCGAG |
| | GACGACAGGAGCGAGGTGGTGGTGG |
| | CCGCCCACGACCTGAGGCCCGGCAC |
| | CGCCCTGACCCCCGGCGACGTGAGG |
| | CTGGAGAAGAGGAGCGCCACCACCC |
| | TGCCCGACGGCAGCCAGGCCGACCT |
| | GGACGCCGTGGTGGGCAGCACCCTG |
| | GCCAGCCCCACCAGGAGGGGCGAGG |
| | TGCTGACCGACGTGAGGCTGCTGGG |
| | CAGCAGGCTGGCCGAGAGCACCGCC |
| | GGCCCCGACGCCAGGATCGTGCCCC |
| | TGCACCTGGCCGACAGCGCCCTGGT |
| | GGACCTGGTGAGGGTGGGCGACGTG |
| | GTGGACGTGCTGGCCGCCCCCGTGA |
| | CCGACAGCCCCGCCGCCCTGAGGCT |
| | GCTGGCCACCGACGCCATCGTGGTG |
| | CTGGTGAGCGCCCAGCAGAAGGCCC |
| | AGGCCGCCGACAGCGACAGGGTGGT |
| | GCTGGTGGCCCTGCCCGCCAGGCTG |
| | GCCAACACCGTGGCCGGCGCCGCCC |
| | TGGGCCAGACCGTGACCCTGACCCT |
| | GCACTGA |
| | (SEQ ID NO: 64) |
| | VAESSLNPSLVSRISAFLRPDWTRT |
| | VRARRFAAAGLVMLAGVAALRSNPE |
| | DDRSEVVVAAHDLRPGTALTPGDVR |
| | LEKRSATTLPDGSQADLDAVVGSTL |
| | ASPTRRGEVLTDVRLLGSRLAESTA |
| | GPDARIVPLHLADSALVDLVRVGDV |
| | VDVLAAPVTDSPAALRLLATDAIVV |
| | LVSAQQKAQAADSDRVVLVALPARL |
| | ANTVAGAALGQTVTLTLH |
| | (SEQ ID NO: 65) |
| Rv0995 | ATGGCCGTGGGCCCCCTGAGGGTGA |
| | GCGCCGGCGTGATCAGGCTGAGGCC |
| | CGTGAGGATGAGGGACGGCGTGCAC |
| | TGGAGCAGGATCAGGCTGGCCGACA |
| | GGGCCCACCTGGAGCCCTGGGAGCC |
| | CAGCGCCGACGGCGAGTGGACCGTG |
| | AGGCACACCGTGGCCGCCTGGCCCG |
| | CCGTGTGCAGCGGCCTGAGGAGCGA |
| | GGCCAGGAACGGCAGGATGCTGCCC |
| | TACGTGATCGAGCTGGACGGCCAGT |
| | TCTGCGGCCAGCTGACCATCGGCAA |
| | CGTGACCCACGGCGCCCTGAGGAGC |
| | GCCTGGATCGGCTACTGGGTGCCCA |
| | GCGCCGCCACCGGCGGCGGCGTGGC |
| | CACCGGCGCCCTGGCCCTGGGCCTG |
| | GACCACTGCTTCGGCCCCGTGATGC |
| | TGCACAGGGTGGAGGCCACCGTGAG |
| | GCCCGAGAACGCCGCCAGCAGGGCC |
| | GTGCTGGCCAAGGTGGGCTTCAGGG |
| | AGGAGGGCCTGCTGAGGAGGTACCT |
| | GGAGGTGGACAGGGCCTGGAGGGAC |
| | CACCTGCTGATGGCCATCACCGTGG |
| | AGGAGGTGTACGGCAGCGTGGCCAG |
| | CACCCTGGTGAGGGCCGGCCACGCC |
| | AGCTGGCCCTGA |
| | (SEQ ID NO: 66) |
| | MAVGPLRVSAGVIRLRPVRMRDGVH |
| | WSRIRLADRAHLEPWEPSADGEWTV |
| | RHTVAAWPAVCSGLRSEARNGRMLP |
| | YVIELDGQFCGQLTIGNVTHGALRS |
| | AWIGYWVPSAATGGGVATGALALGL |
| | DHCFGPVMLHRVEATVRPENAASRA |
| | VLAKVGFREEGLLRRYLEVDRAWRD |
| | HLLMAITVEEVYGSVASTLVRAGHA |
| | SWP |
| | (SEQ ID NO: 67) |

In some embodiments, a composition comprises at least two of the variable antigens. In some embodiments, the composition comprises at least three of the variable antigens. In some embodiments, the composition comprises at least four of the variable antigens. In some embodiments, the composition comprises at least five of the variable antigens. In some embodiments, the composition comprises all six variable antigens. In some embodiments, the composition comprises from at least two to six of the variable antigens. In some embodiments, the composition comprises from at least three to six of the variable antigens. In some embodiments, the composition comprises from at least four to six of the variable antigens. In some embodiments, the composition comprises five or six of the variable antigens. In some embodiments, the composition further comprises a pharmaceutically acceptable carrier.

In some embodiments, the fusion protein comprises at least two of the variable antigens. In some embodiments, the fusion protein comprises at least three of the variable antigens. In some embodiments, the fusion protein comprises at least four of the variable antigens. In some embodiments, the fusion protein comprises at least five of the variable antigens. In some embodiments, the fusion protein comprises all six variable antigens. In some embodiments, the fusion protein comprises from at least two to six of the variable antigens. In some embodiments, the fusion protein comprises from at least three to six of the variable antigens. In some embodiments, the fusion protein comprises from at least four to six of the variable antigens. In some embodiments, the fusion protein comprises five or six of the variable antigens.

In some embodiments, the fusion protein comprises Rv2719c, Rv0010c, and Rv1872c. In some embodiments, the fusion protein comprises Rv0012, Rv0990c, and Rv0995.

In any of the embodiments of fusion proteins set forth herein, the individual variable antigens can be present in any order. For example, for a fusion protein comprising Rv0012, Rv0990c, and Rv0995 antigens, the first (or N-terminal) antigen may be Rv0012, Rv0990c, or Rv0995; the second antigen may be Rv0012, Rv0990c, or Rv0995 (whichever one is not the first variable antigen); and the third antigen may be Rv0012, Rv0990c, or Rv0995 (whichever one is not the first or second variable antigen). Likewise for every fusion protein disclosed herein.

Individual variable antigens may be linked together in a C-terminus to N-terminus or N-terminus to C-terminus manner without any linker. Alternately, a linker may be present between any two variable antigens within any of the fusion proteins disclosed herein. In some embodiments, the linker is a segment of DNA or RNA optionally containing one or more restrictions sites, wherein the linker is inserted between nucleic acid molecules encoding two variable antigens of any of the fusion proteins disclosed herein.

In some embodiments, the fusion protein comprises Rv2719c-Rv0010c-Rv1872c (Construct F; see Table 8). The nucleotide sequence is SEQ ID NO:68, and the corresponding amino acid sequence is SEQ ID NO:69 (including a C-terminal HA tag (YPYDVPDYA; SEQ ID NO:17) added).

In some embodiments, the fusion protein comprises Rv0012-Rv0990c-Rv0995 (Construct G; see Table 8). The nucleotide sequence is SEQ ID NO:70, and the corresponding amino acid sequence is SEQ ID NO:71 (including a C-terminal HA tag (YPYDVPDYA; SEQ ID NO:17) added).

TABLE 8
Variable Antigen Cassette

| | nucleotide sequence |
|---|---|
| Construct | amino acid sequence |
| F | ATGACCCCCGTGAGGCCCCCCCACA |
| | CCCCCGACCCCCTGAACCTGAGGGG |
| | CCCCCTGGACGGCCCCAGGTGGAGG |
| | AGGGCCGAGCCCGCCCAGAGCAGGA |
| | GGCCCGGCAGGAGCAGGCCCGGCGG |
| | CGCCCCCCTGAGGTACCACAGGACC |
| | GGCGTGGGCATGAGCAGGACCGGCC |
| | ACGGCAGCAGGCCCGTGCCCCCCGC |
| | CACCACCGTGGGCCTGGCCCTGCTG |
| | GCCGCCGCCATCACCCTGTGGCTGG |
| | GCCTGGTGGCCCAGTTCGGCCAGAT |
| | GATCACCGGCGGCAGCGCCGACGGC |
| | AGCGCCGACAGCACCGGCAGGGTGC |
| | CCGACAGGCTGGCCGTGGTGAGGGT |
| | GGAGACCGGCGAGAGCCTGTACGAC |
| | GTGGCCGTGAGGGTGGCCCCCAACG |
| | CCCCCACCAGGCAGGTGGCCGACAG |
| | GATCAGGGAGCTGAACGGCCTGCAG |
| | ACCCCCGCCCTGGCCGTGGGCCAGA |
| | CCCTGATCGCCCCCGTGGGCATGCA |
| | GCAGACCGCCTGGGCCCCCAGGACC |
| | AGCGGCATCGCCGGCTGCGGCGCCG |
| | GCGGCGTGGTGATGGCCATCGCCAG |
| | CGTGACCCTGGTGACCGACACCCCC |
| | GGCAGGGTGCTGACCGGCGTGGCCG |
| | CCCTGGGCCTGATCCTGTTCGCCAG |
| | CGCCACCTGGAGGGCCAGGCCCAGG |
| | CTGGCCATCACCCCCGACGGCCTGG |
| | CCATCAGGGGCTGGTTCAGGACCCA |
| | GCTGCTGAGGCACAGCAACATCAAG |
| | ATCATCAGGATCGACGAGTTCAGGA |
| | GGTACGGCAGGCTGGTGAGGCTGCT |
| | GGAGATCGAGACCGTGAGCGGCGGC |
| | CTGCTGATCCTGAGCAGGTGGGACC |
| | TGGGCACCGACCCCGTGGAGGTGCT |
| | GGACGCCCTGACCGCCGCCGGCTAC |
| | GCCGGCAGGGGCCAGAGGATGGCCG |
| | TGAACAGGAGGGTGCCCAGGGTGAG |
| | GGACCTGGCCCCCCTGCTGCAGTTC |
| | AACAGGCCCCAGTTCGACACCAGCA |
| | AGAGGAGGCTGGGCGCCGCCCTGAC |
| | CATCCAGGACCTGAGGAGGATCGCC |
| | AAGAGGAGGACCCCCAGGGCCGCCT |
| | TCGACTACGCCGACGGCGGCGCCGA |
| | GGACGAGCTGAGCATCGCCAGGGCC |
| | AGGCAGGGCTTCAGGGACATCGAGT |
| | TCCACCCCACCATCCTGAGGGACGT |
| | GACCACCGTGTGCGCCGGCTGGAAC |
| | GTGCTGGGCCAGCCCACCGTGCTGC |
| | CCTTCGGCATCGCCCCCACCGGCTT |
| | CACCAGGCTGATGCACACCGAGGGC |
| | GAGATCGCCGGCGCCAGGGCCGCCG |
| | CCGCCGCCGGCATCCCCTTCAGCCT |
| | GAGCACCCTGGCCACCTGCGCCATC |
| | GAGGACCTGGTGATCGCCGTGCCCC |
| | AGGGCAGGAAGTGGTTCCAGCTGTA |
| | CATGTGGAGGGACAGGGACAGGAGC |
| | ATGGCCCTGGTGAGGAGGGTGGCCG |
| | CCGCCGGCTTCGACACCATGCTGGT |
| | GACCGTGGACGTGCCCGTGGCCGGC |
| | GCCAGGCTGAGGGACGTGAGGAACG |
| | GCATGAGCATCCCCCCCGCCCTGAC |
| | CCTGAGGACCGTGCTGGACGCCATG |
| | GGCCACCCCAGGTGGTGGTTCGACC |
| | TGCTGACCACCGAGCCCCTGGCCTT |
| | CGCCAGCCTGGACAGGTGGCCCGGC |
| | ACCGTGGGCGAGTACCTGAACACCG |
| | TGTTCGACCCCAGCCTGACCTTCGA |
| | CGACCTGGCCTGGATCAAGAGCCAG |
| | TGGCCCGGCAAGCTGGTGGTGAAGG |
| | GCATCCAGACCCTGGACGACGCCAG |
| | GGCCGTGGTGGACAGGGGCGTGGAC |
| | GGCATCGTGCTGAGCAACCACGGCG |
| | GCAGGCAGCTGGACAGGGCCCCCGT |
| | GCCCTTCCACCTGCTGCCCCACGTG |
| | GCCAGGGAGCTGGGCAAGCACACCG |
| | AGATCCTGGTGGACACCGGCATCAT |
| | GAGCGGCGCCGACATCGTGGCCGCC |
| | ATCGCCCTGGGCGCCAGGTGCACCC |
| | TGATCGGCAGGGCCTACCTGTACGG |
| | CCTGATGGCCGGCGGCGAGGCCGGC |
| | GTGAACAGGGCCATCGAGATCCTGC |
| | AGACCGGCGTGATCAGGACCATGAG |
| | GCTGCTGGGCGTGACCTGCCTGGAG |
| | GAGCTGAGCCCCAGGCACGTGACCC |
| | AGCTGAGGAGGCTGGGCCCCATCGG |
| | CGCCCCCACCTACCCCTACGACGTG |
| | CCCGACTACGCCTGA |
| | (SEQ ID NO: 68) |
| | MTPVRPPHTPDPLNLRGPLDGPRWR |
| | RAEPAQSRRPGRSRPGGAPLRYHRT |
| | GVGMSRTGHGSRPVPPATTVGLALL |
| | AAAITLWLGLVAQFGQMITGGSADG |
| | SADSTGRVPDRLAVVRVETGESLYD |
| | VAVRVAPNAPTRQVADRIRELNGLQ |
| | TPALAVGQTLIAPVGMQQTAWAPRT |
| | SGIAGCGAGGVVMAIASVTLVTDTP |
| | GRVLTGVAALGLILFASATWRARPR |
| | LAITPDGLAIRGWFRTQLLRHSNIK |
| | IIRIDEFRRYGRLVRLLEIETVSGG |
| | LLILSRWDLGTDPVEVLDALTAAGY |
| | AGRGQRMAVNRRVPRVRDLAPLLQF |
| | NRPQFDTSKRRLGAALTIQDLRRIA |
| | KRRTPRAAFDYADGGAEDELSIARA |
| | EGEIAGARAAAAAGIPFSLSTLATC |
| | AIEDLVIAVPQGRKWFQLYMWRDRD |
| | RSMALVRRVAAAGFDTMLVTVDVPV |
| | AGARLRDVRNGMSIPPALTLRTVLD |
| | AMGHPRWWFDLLTTEPLAFASLDRW |
| | PGTVGEYLNTVFDPSLTFDDLAWIK |
| | SQWPGKLVVKGIQTLDDARAVVDRG |
| | VDGIVLSNHGGRQLDRAPVPFHLLP |
| | HVARELGKHTEILVDTGIMSGADIV |
| | AAIALGARCTLIGRAYLYGLMAGGE |
| | AGVNRAIEILQTGVIRTMRLLGVTC |
| | LEELSPRHVTQLRRLGPIGAPTYPY |
| | DVPDYA |
| | (SEQ ID NO: 69) |
| G | ATGAGGCTGACCCACCCCACCCCCT |
| | GCCCCGAGAACGGCGAGACCATGAT |
| | CGACAGGAGGAGGAGCGCCTGGAGG |
| | TTCAGCGTGCCCCTGGTGTGCCTGC |
| | TGGCCGGCCTGCTGCTGGCCGCCAC |
| | CCACGGCGTGAGCGGCGGCACCGAG |
| | ATCAGGAGGAGCGACGCCCCCAGGC |
| | TGGTGGACCTGGTGAGGAGGGCCCA |
| | GGCCAGCGTGAACAGGCTGGCCACC |
| | GAGAGGGAGGCCCTGACCACCAGGA |
| | TCGACAGCGTGCACGGCAGGAGCGT |
| | GGACACCGCCCTGGCCGCCATGCAG |
| | AGGAGGAGCGCCAAGCTGGCCGGCG |
| | TGGCCGCCATGAACCCCGTGCACGG |
| | CCCCGGCCTGGTGGTGACCCTGCAG |
| | GACGCCCAGAGGGACGCCAACGGCA |
| | GGTTCCCCAGGGACGCCAGCCCCGA |
| | CGACCTGGTGGTGCACCAGCAGGAC |
| | ATCGAGGCCGTGCTGAAC-GCCCTG |
| | TGGAACGCCGGCGCCGAGGCCATCC |
| | AGATGCAGGACCAGAGGATCATCGC |
| | CATGAGCATCGCCAGGTGCGTGGGC |
| | AACACCCTGCTGCTGAACGGCAGGA |
| | CCTACAGCCCCCCCTACACCATCGC |
| | CGCCATCGGCGACGCCGCCGCCATG |
| | CAGGCCGCCCTGGCCGCCGCCCCCC |
| | TGGTGACCCTGTACAAGCAGTACGT |
| | GGTGAGGTTCGGCCTGGGCTACTGC |
| | GAGGAGGTGCACCCCGACCTGCAGA |
| | TCGTGGGCTACGCCGACCCCGTGAG |
| | GATGCACTTCGCCCAGCCCGCCGGC |
| | CCCCTGGACTACGTGGCCGAGAGCA |
| | GCCTGAACCCCAGCCTGGTGAGCAG |
| | GATCAGCGCC7TCCTGAGGCCCGAC |
| | TGGACCAGGACCGTGAGGGCCAGGA |
| | GGTTCGCCGCCGCCGGCCTGGTGAT |
| | GCTGGCCGGCGTGGCCGCCCTGAGG |
| | AGCAACeCCGAGGACGACAGGAGCG |
| | AGGTGGTGGTGGCCGCCCACGACCT |
| | GAGGCCCGGCACCGCCCTGACCCCC |
| | GGCGACGTGAGGCTGGAGAAGAGGA |
| | GCGCCACCACCCTGCCCGACGGCAG |
| | CCAGGCCGACCTGGACGCCGTGGTG |
| | GGCAGCACCCTGGCCAGCCCCACCA |
| | GGAGGGGCGAGGTGCTGACCGACGT |
| | GAGGCTGCTGGGCAGCAGGCTGGCC |
| | GAGAGCACCGCCGGCCCCGACGCCA |
| | GGATCGTGCCCCTGCACCTGGCCGA |
| | CAGCGCCCTGGTGGACCTGGTGAGG |
| | GTGGGCGACGTGGTGGACGTGCTGG |
| | CCGCCCCCGTGACCGACAGCCCCGC |
| | CGCCCTGAGGCTGCTGGCCACCGAC |
| | GCCATCGTGGTGCTGGTGAGCGCCC |
| | AGCAGAAGGCCCAGGCCGCCGACAG |
| | CGACAGGGTGGTGCTGGTGGCCCTG |
| | CCCGCCAGGCTGGCCAACACCGTGG |
| | CCGGCGCCGCCCTGGGCCAGACCGT |
| | GACCCTGACCCTGCACATGGCCGTG |
| | GGCCCCCTGAGGGTGAGCGCCGGCG |
| | TGATCAGGCTGAGGCCCGTGAGGAT |
| | GAGGGACGGCGTGCACTGGAGCAGG |
| | ATCAGGCTGGCCGACAGGGCCCACC |
| | TGGAGCCCTGGGAGCCCAGCGCCGA |
| | CGGCGAGTGGACCGTGAGGCACACC |
| | GTGGCCGCCTGGCCCGCCGTGTGCA |
| | GCGGCCTGAGGAGCGAGGCCAGGAA |
| | CGGCAGGATGCTGCCCTACGTGATC |
| | GAGCTGGACGGCCAGTTCTGCGGCC |
| | AGCTGACCATCGGCAACGTGACCCA |
| | CGGCGCCCTGAGGAGCGCCTGGATC |
| | GGCTACTGGGTGCCCAGCGCCGCCA |
| | CCGGCGGCGGCGTGGCCACCGGCGC |
| | CCTGGCCCTGGGCCTGGACCACTGC |
| | TTCGGCCCCGTGATGCTGCACAGGG |
| | TGGAGGCCACCGTGAGGCCCGAGAA |
| | CGCCGCCAGCAGGGCCGTGCTGGCC |
| | AAGGTGGGCTTCAGGGAGGAGGGCC |
| | TGCTGAGGAGGTACCTGGAGGTGGA |
| | CAGGGCCTGGAGGGACCACCTGCTG |
| | ATGGCCATCACCGTGGAGGAGGTGT |
| | ACGGCAGCGTGGCCAGCACCCTGGT |
| | GAGGGCCGGCCACGCCAGCTGGCCC |
| | TACCCCTACGACGTGCCCGACTACG |
| | CCTGA |
| | (SEQ ID NO: 70) |
| | MRLTHPTPCPENGETMIDRRRSAWR |
| | FSVPLVCLLAGLLLAATHGVSGGTE |
| | IRRSDAPRLVDLVRRAQASVNRLAT |
| | EREALTTRIDSVHGRSVDTALAAMQ |
| | RRSAKLAGVAAMNPVHGPGLVVTLQ |
| | DAQRDANGRFPRDASPDDLVVHQQD |
| | IEAVLNALWNAGAEAIQMQDQRIIA |
| | MSIARCVGNTLLLNGRTYSPPYTIA |
| | AIGDAAAMQAALAAAPLVTLYKQYV |
| | VRFGLGYCEEVHPDLQIVGYADPVR |
| | MHFAQPAGPLDYVAESSLNPSLVSR |
| | ISAFLRPDWTRTVRARRFAAAGLVM |
| | LAGVAALRSNPEDDRSEVVVAAHDL |
| | RPGTALTPGDVRLEKRSATTLPDGS |
| | QADLDAVVGSTLASPTRRGEVLTDV |
| | RLLGSRLAESTAGPDARIVPLHLAD |
| | SALVDLVRVGDVVDVLAAPVTDSPA |
| | ALRLLATDAIVVLVSAQQKAQAADS |
| | DRVVLVALPARLANTVAGAALGQTV |
| | TLTLHMAVGPLRVSAGVIRLRPVRM |
| | RDGVHWSRIRLADRAHLEPWEPSAD |
| | GEWTVRHTVAAWPAVCSGLRSEARN |
| | GRMLPYVIELDGQFCGQLTIGNVTH |
| | GALRSAWIGYWVPSAATGGGVATGA |
| | LALGLDHCFGPVMLHRVEATVRPEN |
| | AASRAVLAKVGFREEGLLRRYLEVD |
| | RAWRDHLLMAITVEEVYGSVASTLV |
| | RAGHASWPYPYDVPDYA |
| | (SEQ ID NO: 71) |

Any Mtb antigen, including any Mtb antigen within any of the fusion proteins described herein, can have an amino acid sequence that is 100%, or from 70% to 99.9%, identical to the particular amino acid sequence listed in Tables 1 through 8. The amino acid sequence of any individual Mtb antigen, including any Mtb antigen within any of the fusion proteins described herein, can be at least 70%, at least 75%, at least 80%, at least 85%, at least 90%, at least 95%, at least 96%, at least 97%, at least 98%, or at least 99% identical to the particular amino acid sequence listed in Tables 1 through 8. Identity or similarity with respect to an amino acid or nucleotide sequence is defined herein as the percentage of amino acid residues (or nucleotide residues as the case may be) in the particular Mtb antigen that are identical (i.e., same residue) with the amino acid or nucleotide sequence for the Mtb antigen shown in Tables 1 through 8, after aligning the sequences and introducing gaps, if necessary, to achieve the maximum percent sequence identity. Percent sequence identity can be determined by, for example, the Gap program (Wisconsin Sequence Analysis Package, Version 8 for Unix, Genetics Computer Group, University Research Park, Madison WI), using default settings, which uses the algorithm of Smith and Waterman (Adv. Appl. Math., 1981, 2, 482-489). Any amino acid number calculated as a % identity can be rounded up or down, as the case may be, to the closest whole number.

Any Mtb antigen, including any Mtb antigen within any of the fusion proteins described herein, can be fragments of the particular amino acid sequences listed in Tables 1, 3, 5, and 7. The amino acid sequence of any individual Mtb antigen, including any Mtb antigen within any of the fusion proteins described herein, can be missing consecutive amino acids constituting at least 20%, at least 15%, at least 10%, at least 5%, at least 4%, at least 3%, at least 2%, or at least 1%, of the particular amino acid sequences listed in Tables 1, 3, 5, and 7. The omitted consecutive amino acids may be from the C-terminus or N-terminus portion of the antigen. Alternately, the omitted consecutive amino acids may be from the internal portion of the antigen, thus retaining at least its C-terminus and N-terminus amino acids of the antigen.

Any Mtb antigen, including any Mtb antigen within any of the fusion proteins described herein, can have one or more amino acid additions, deletions, or substitutions compared to the particular amino acid sequences listed in Tables 1 through 8. Any individual Mtb antigen, including any Mtb antigen within any of the fusion proteins described herein, can have at least one, at least two, at least three, at least four, at least five, at least six, at least seven, at least eight, at least nine, at least ten, at least eleven, or at least twelve amino acid additions, deletions, or substitutions compared to the particular amino acid sequences listed in Tables 1 through 8. Any individual Mtb antigen, including any Mtb antigen within any of the fusion proteins described herein, can have one, two, three, four, five, six, seven, eight, nine, ten, eleven, or twelve amino acid additions, deletions, or substitutions compared to the particular amino acid sequences listed in Tables 1 through 8. The amino acid additions, deletions, or substitutions can take place at any amino acid position within the Mtb antigen.

Where a particular Mtb antigen, including any Mtb antigen within any of the fusion proteins described herein, comprises at least one or more substitutions, the substituted amino acid(s) can each be, independently, any naturally occurring amino acid or any non-naturally occurring amino acid. Thus, a particular Mtb antigen may comprise one or more amino acid substitutions that are naturally occurring amino acids and/or one or more amino acid substitutions that are non-naturally occurring amino acids. Individual amino acid substitutions are selected from any one of the following: 1) the set of amino acids with nonpolar sidechains, for example, Ala, Cys, Ile, Leu, Met, Phe, Pro, Val; 2) the set of amino acids with negatively charged side chains, for example, Asp, Glu; 3) the set of amino acids with positively charged sidechains, for example, Arg, His, Lys; and 4) the set of amino acids with uncharged polar sidechains, for example, Asn, Cys, Gln, Gly, His, Met, Phe, Ser, Thr, Trp, Tyr, to which are added Cys, Gly, Met and Phe. Substitutions of a member of one class with another member of the same class are contemplated herein. Naturally occurring amino acids include, for example, alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val). Non-naturally occurring amino acids include, for example, norleucine, omithine, norvaline, homoserine, and other amino acid residue analogues such as those described in Ellman et al., Meth. Enzym., 1991, 202, 301-336. To generate such non-naturally occurring amino acid residues, the procedures of Noren et al., Science, 1989, 244, 182 and Ellman et al., supra, can be used. Briefly, these procedures involve chemically activating a suppressor tRNA with a non-naturally occurring amino acid residue followed by in vitro transcription and translation of the RNA.

The Mtb antigens, including any Mtb antigen within any of the fusion proteins described herein, which are modified as described herein retain their ability to elicit an immune response against Mycobacterium tuberculosis. That is, modification of a particular Mtb antigen, including any Mtb antigen within any of the fusion proteins described herein, will still allow the resultant Mtb antigen, or fusion protein comprising the same, to elicit an immune response against Mycobacterium tuberculosis.

The present disclosure also provides nucleic acid molecules encoding any of the fusion proteins described herein that comprise at least two Mtb antigens. The nucleic acid molecules described herein and in Tables 1 through 8 are representative. The specific sequences recited in

Tables 1 through 8 are simply representative examples of nucleic acid molecules that can encode a particular Mtb antigen within a fusion protein. One skilled in the art having knowledge of the genetic code can routinely prepare and design a plethora of nucleic acid molecules encoding the same Mtb antigen. The length and nucleotide content of any particular nucleic acid molecule is dictated by the desired amino acid sequence of the encoded Mtb antigen. The nucleic acid molecule sequences shown in Tables 1 through 8 are DNA, although RNA nucleic acid molecules are also contemplated.

The present disclosure also provides vectors encoding any of the Mtb antigens, including Mtb antigens within any of the fusion proteins described herein, including any of the modified versions described herein. The vector can be capable of expressing an Mtb antigen in the cell of a mammal in a quantity effective to elicit an immune response in the mammal. The vector can be recombinant. The vector can comprise heterologous nucleic acid encoding the antigen. The vector can be a plasmid. In some embodiments, the plasmid is a DNA plasmid, such as a pVAX backbone vector. The vector can be useful for transfecting cells with nucleic acid encoding an Mtb antigen, which the transformed host cell is cultured and maintained under conditions wherein expression of the antigen takes place.

In some embodiments, coding sequences can be optimized for stability and high levels of expression. In some instances, codons are selected to reduce secondary structure formation of the RNA such as that formed due to intramolecular bonding.

In some embodiments, the vectors can comprise regulatory elements for gene expression of the coding sequences of the nucleic acid. The regulatory elements can be a promoter, an enhancer an initiation codon, a stop codon, or a polyadenylation signal. In some embodiments, the vector can comprise heterologous nucleic acid encoding an Mtb antigen and can further comprise an initiation codon, which is upstream of the antigen coding sequence, and a stop codon, which is downstream of the antigen coding sequence. The initiation and termination codon are in frame with the antigen coding sequence.

The vector can also comprise a promoter that is operably linked to the antigen coding sequence. The promoter operably linked to the Mtb antigen coding sequence can be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter, or the like. The promoter can also be a promoter from a human gene such as human actin, human myosin, human hemoglobin, human muscle creatine, or human metalothionein. The promoter can also be a tissue specific promoter, such as a muscle or skin specific promoter, natural or synthetic. Representative examples of promoters include the bacteriophage T7 promoter, bacteriophage T3 promoter, SP6 promoter, lac operator-promoter, tac promoter, mycobacterial Hsp60 promoter, SV40 late promoter, SV40 early promoter, RSV-LTR promoter, CMV IE promoter, SV40 early promoter or SV40 late promoter and the CMV IE promoter.

The vector can also comprise a polyadenylation signal, which can be downstream of the antigen coding sequence. The polyadenylation signal can be a SV40 polyadenylation signal, LTR polyadenylation signal, CMV polyadeylation signal, bovine growth hormone (bGH) polyadenylation signal, human growth hormone (hGH) polyadenylation signal, or human β-globin polyadenylation signal. The SV40 polyadenylation signal can be a polyadenylation signal from a pCEP4 vector (Invitrogen, San Diego, CA).

The vector can also comprise an enhancer upstream of the consensus BONT-A, BoNT-B, BONT-E, and BoNT-F antigen sequences. The enhancer can be necessary for DNA expression. The enhancer can be human actin, human myosin, human hemoglobin, human muscle creatine or a viral enhancer such as one from CMV, HA, RSV or EBV. Polynucleotide function enhancers are described in U.S. Pat. Nos. 5,593,972, 5,962,428, and WO94/016737.

The vector can also comprise a mammalian origin of replication in order to maintain the vector extrachromosomally and produce multiple copies of the vector in a cell. The vector can be pVAX1, pCEP4 or pREP4 from Invitrogen (San Diego, CA), which can comprise the Epstein Barr virus origin of replication and nuclear antigen EBNA-1 coding region, which can produce high copy episomal replication without integration. The vector can be pVAX1 or a pVax1 variant with changes such as the variant plasmid described herein. The variant pVax1 plasmid is a 2998 basepair variant of the backbone vector plasmid pVAX1 (Invitrogen, Carlsbad CA). The CMV promoter is located at bases 137-724. The T7 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993.

The vector can also comprise a regulatory sequence, which can be well suited for gene expression in a mammalian or human cell into which the vector is administered. The consensus coding sequence can comprise a codon, which can allow more efficient transcription of the coding sequence in the host cell.

The vector can be pSE420 (Invitrogen, San Diego, Calif.) or pET28b (EMD Millipore, Billerca, Mass.), which can be used for protein production in Escherichia coli (E. coli). The vector can also be pYES2 (Invitrogen, San Diego, Calif.), which can be used for protein production in Saccharomyces cerevisiae strains of yeast. The vector can also be of the MAXBAC™ complete baculovirus expression system (Invitrogen, San Diego, Calif.), which can be used for protein production in insect cells. The vector can also be pcDNA I or pcDNA3 (Invitrogen, San Diego, Calif.), which may be used for protein production in mammalian cells such as Chinese hamster ovary (CHO) cells. The vector can be expression vectors or systems to produce protein by routine techniques and readily available starting materials including Sambrook et al., Molecular Cloning and Laboratory Manual, Second Ed., Cold Spring Harbor (1989).

In some embodiments, the vector is a viral vector. Suitable viral vectors include, but are not limited to, an adenovirus vector, an adeno-associated virus vector, a poxvirus vector (such as, for example, vaccinia virus vector), a paramyxovirus vector, a fowlpox virus vector, an attenuated yellow fever vectors (such as, for example, YFV-17D), an alphavirus vector, a retrovirus vector (such as, for example, lentivirus vector), a Sendai virus vector, and cytomegalovirus (CMV) vector. Suitable adenovirus vectors include, but are not limited to, adenovirus 4, adenovirus 5, chimpanzee adenovirus 3, chimpanzee adenovirus 63, and chimpanzee adenovirus 68. A suitable vaccinia virus vector includes, but is not limited to, modified vaccinia Ankara (MVA). Suitable paramyxovirus vectors include, but are not limited to, modified parainfluenza virus (PIV2) and recombinant human parainfluenza virus (rHPIV2). Suitable CMV vectors include, but are not limited to, Rhesus Macaque CMV (RhCMV) vectors and Human CMV (HCMV) vectors. In some embodiments, the vector is present within a composition comprising a pharmaceutically acceptable carrier. One skilled in the art is readily familiar with numerous vectors, many of which are commercially available.

In some embodiments, the vector is a non-viral vector. In some embodiments, the non-viral vector is RNA, such as mRNA. In some embodiments, the mRNA is protamine-complexed mRNA, wherein the Mtb antigen or fusion protein is encoded by the mRNA, and the protamine complexes contribute a strong immunostimulatory signal. An exemplary mRNA vector platform is RNActive® (Cure Vac Inc).

The present disclosure also provides host cells comprising any of the nucleic acid molecules or vectors disclosed herein. The host cells can be used, for example, to express the Mtb antigens, or fragments of thereof. The Mtb antigens, or fragments thereof, can also be expressed in cells in vivo. The host cell that is transformed (for example, transfected) to produce the Mtb antigens, or fragments of thereof can be an immortalised mammalian cell line, such as those of lymphoid origin (for example, a myeloma, hybridoma, trioma or quadroma cell line). The host cell can also include normal lymphoid cells, such as B-cells, that have been immortalized by transformation with a virus (for example, the Epstein-Barr virus).

In some embodiments, the host cells include, but are not limited to: bacterial cells, such as E. coli, Caulobacter crescentus, Streptomyces species, and Salmonella typhimurium; yeast cells, such as Saccharomyces cerevisiae, Schizosaccharomyces pombe, Pichia pastoris, Pichia methanolica; insect cell lines, such as those from Spodoptera frugiperda (for example, Sf9 and Sf21 cell lines, and expresSF™ cells (Protein Sciences Corp., Meriden, CT, USA)), Drosophila S2 cells, and Trichoplusia in High Five® Cells (Invitrogen, Carlsbad, CA, USA); and mammalian cells, such as COS1 and COS7 cells, Chinese hamster ovary (CHO) cells, NSO myeloma cells, NIH 3T3 cells, 293 cells, Procell92S, perC6, HEPG2 cells, HeLa cells, L cells, HeLa, MDCK, HEK293, WI38, murine ES cell lines (for example, from strains 129/SV, C57/BL6, DBA-1, 129/SVJ), K562, Jurkat cells, and BW5147. Other useful mammalian cell lines are well known and readily available from the American Type Culture Collection (“ATCC”) (Manassas, VA, USA) and the National Institute of General Medical Sciences (NIGMS) Human Genetic Cell Repository at the Coriell Cell Repositories (Camden, NJ, USA). In some embodiments, the cell is a recombinant BCG. These cell types are only representative and are not meant to be an exhaustive list.

Among other considerations, some of which are described above, a host cell strain may be chosen for its ability to process the expressed Mtb antigens, or fragment thereof, in the desired fashion. Post-translational modifications of the polypeptide include, but are not limited to, glycosylation, acetylation, carboxylation, phosphorylation, lipidation, and acylation, and it is an aspect of the present disclosure to provide Mtb antigens thereof with one or more of these post-translational modifications.

In some embodiments, the recombinant BCG has been genetically engineered to express a functional endosomalytic protein that is bioactive at pH values near neutrality (e.g. about pH 6-8 or about 6.5 to 7.5). The endosomalytic protein is active within Mycobacteria-containing endosomes, which typically have an internal pH near neutrality. The activity of the endosomalytic protein produced by the rBCG results in disruption of the endosome, permitting the rBCG to escape from the endosome and into the cytoplasm of the cell. In some embodiments, the endosomalytic protein that is introduced into the rBCG by genetic engineering is Perfringolysin O (PfoA) from Clostridium perfringens or a mutant thereof, such as PfoA_{G137Q}, as described in WO 2007/058663.

In some embodiments, the Mycobacteria are attenuated, as exemplified by BCG. However, those of skill in the art will recognize that other attenuated and nonattenuated Mycobacteria exist which would also be suitable for use herein. Examples of additional types of Mycobacteria include, but are not limited to, M. tuberculosis strain CDC1551, M. tuberculosis strain Beijing, M. tuberculosis strain H37Ra (ATCC #: 25177), M. tuberculosis strain H37Rv (ATCC #: 25618), M. bovis (ATCC #: 19211 and 27291), M. fortuitum (ATCC #: 15073), M. smegmatis (ATCC #: 12051 and 12549), M. intracellulare (ATCC #: 35772 and 13209), M. kansasii (ATCC #: 21982 and 35775) M. avium (ATCC #: 19421 and 25291), M. gallinarum (ATCC #: 19711), M. vaccae (ATCC #: 15483 and 23024), M. leprae (ATCC #: ), M. marinarum (ATCC #: 11566 and 11567), and M. microtti (ATCC #: 11152).

Examples of attenuated Mycobacterium strains include, but are not restricted To, M. tuberculosis pantothenate auxotroph strain, M. tuberculosis rpoV mutant strain, M. tuberculosis leucine auxotroph strain, BCG Danish strain (ATCC #35733), BCG Japanese strain (ATCC #35737), BCG Chicago strain (ATCC #27289), BCG Copenhagen strain (ATCC #: 27290), BCG Pasteur strain (ATCC #: 35734), BCG Glaxo strain (ATCC #: 35741), BCG Connaught strain (ATCC #35745), BCG Montreal (ATCC #35746), BCG1331 strain, BCG Tokyo strain, BCG Moreau strain, BCG-Pasteur Aeras, and BCG Moscow strain.

In some embodiments, the cell comprising the one or more vector(s) is present within a composition comprising a pharmaceutically acceptable carrier.

In some embodiments, the Mtb antigen, or fragment thereof, is labeled with a detectable marker. Detectable markers include, but are not limited to, radioactive isotopes (such as P³² and S³⁵), enzymes (such as horseradish peroxidase, chloramphenicol acetyltransferase (CAT), β-galactosidase (β-gal), and the like), fluorochromes, chromophores, colloidal gold, dyes, and biotin. The labeled Mtb antigens, or fragments thereof, can be used to carry out diagnostic procedures in a variety of cell or tissue types. For imaging procedures, in vitro or in vivo, the Mtb antigens can be labeled with additional agents, such as NMR contrasting agents, X-ray contrasting agents, or quantum dots. Methods for attaching a detectable agent to polypeptides are known in the art. The Mtb antigens can also be attached to an insoluble support (such as a bead, a glass or plastic slide, or the like).

In some embodiments, the Mtb antigens, or fragment thereof, can be conjugated to a therapeutic agent including, but not limited to, radioisotopes (such as ¹¹¹In or ⁹⁰Y), toxins (such as tetanus toxoid or ricin), toxoids, and chemotherapeutic agents.

In some embodiments, the Mtb antigens, or fragments thereof, can be conjugated to an imaging agent. Imaging agents include, for example, a labeling moiety (such as biotin, fluorescent moieties, radioactive moieties, histidine tag or other peptide tags) for easy isolation or detection.

The present disclosure also provides compositions comprising at least two Mtb antigens or at least one Mtb fusion protein. In some embodiments, the at least two Mtb antigens are not present in a fusion protein. In some embodiments, the at least two Mtb antigens are in the form of a protein and not nucleic acid molecules encoding the Mtb antigens.

The present disclosure also provides any of the Mtb compositions described herein in which the composition comprises at least one nucleic acid molecule encoding at least one of the Mtb antigens. In some embodiments, the composition comprises one Mtb antigen in protein form and one or two nucleic acid molecules encoding two Mtb antigens. In some embodiments, the composition comprises two Mtb antigens in protein form, optionally as a fusion protein, and one nucleic acid molecule encoding one Mtb antigen. Thus, the present composition can be a mixture of a protein Mtb antigen(s) and nucleic acid molecule(s) encoding an Mtb antigen(s) to make up any of the compositions described herein.

In some embodiments, at least two Mtb antigens, or a fusion protein comprising the same, are encoded by one or more nucleic acid molecules within one or more vectors. In some embodiments, the one or more vectors is one or more viral vectors. In some embodiments, the one or more viral vectors are any one or more of an adenovirus vector, an adeno-associated virus vector, a poxvirus vector (such as, for example, vaccinia virus vector), a paramyxovirus vector, a fowlpox virus vector, an attenuated yellow fever vectors (such as, for example, YFV-17D), an alphavirus vector, a retrovirus vector (such as, for example, lentivirus vector), a Sendai virus vector, and CMV vector. In some embodiments, the adenovirus vector is adenovirus 4, adenovirus 5, chimpanzee adenovirus 3, chimpanzee adenovirus 63, or chimpanzee adenovirus 68. In some embodiments, the vaccinia virus vector is MVA. In some embodiments, the paramyxovirus vector is PIV2 or rHPIV2. In some embodiments, the CMV vector is a RhCMV vector of an HCMV vector. In some embodiments, the vector is present within a composition comprising a pharmaceutically acceptable carrier. In some embodiments, the at least two Mtb antigens are encoded by a single nucleic acid molecule within the same expression vector as a fusion protein.

In some embodiments, the one or more vectors is a non-viral vector. In some embodiments, the non-viral vector is RNA, such as mRNA. In some embodiments, the mRNA is protamine-complexed mRNA. An exemplary mRNA vector platform is RNActive® (Cure Vac Inc).

In some embodiments, where a rBCG is used as the vehicle to deliver the Mtb antigens, or fusion proteins, or nucleic acids and or vectors comprising or encoding the same, expression of all or part of the Dos R regulon is not up-regulated in the rBCG. In some embodiments, one or more of the following Dos R regulon antigens are not up-regulated in the rBCG: Rv1738, Rv2623, Rv2031c, Rv2032, Rv2626c, Rv2005c, Rv3127, Rv1733c, Rv1996, Rv2628c, Rv0079, Rv3130c, Rv3131, Rv1813c, Rv2006, Rv2029c, Rv2627c, Rv2030c, Rv3132c, and Rv2629. In some embodiments, the rBCG does not comprise up-regulation of: 1) one or more Mtb antigens, including “classical” Mtb antigens such as 85A, 85B and TB 10.4; and 2) at least one Mtb resuscitation antigen selected from Rv0867c, Rv1009, Rv1884c, Rv2389c, Rv2450c, Rv0288, Rv1009, Rv0685, Rv0824c, Rv1349, Rv2744c, Rv3347c, Rv1130, and Rv1169c. In some embodiments, the rBCG does not include the expression of the following combinations: classical antigens Rv1886c, Rv3804c; resuscitation antigens Rv0867c, Rv1884c, Rv2389c; and Mtb-specific antigen Rv3407. In some embodiments, the rBCG does not include the expression of the following combination: Rv3804c, Rv1886c, and Rv3407, or in addition with Rv3133c, and with the combination of Rv0867c, Rv1884c, and Rv2389c. In some embodiments, the rBCG does not include the expression of the following combination: TB10.4, Ag85B, Ag85A, and Rv3407. In some embodiments, the cell is not a rBCG.

The present disclosure also provides compositions comprising any one or more of the fusion proteins, Mtb antigens, nucleic acid molecules encoding Mtb antigens, including fusion proteins thereof, cells, and/or vectors and a pharmaceutically acceptable carrier. Compositions include, for example, pharmaceutical compositions. A pharmaceutically acceptable carrier refers to at least one component of a pharmaceutical preparation that is normally used for administration of active ingredients. As such, a carrier can contain any pharmaceutical excipient used in the art and any form of vehicle for administration. Carriers include, but are not limited to, phosphate buffered saline, physiological saline, water, citrate/sucrose/Tween formulations and emulsions such as, for example, oil/water emulsions.

The compositions can also include an active therapeutic agent and a variety of other pharmaceutically acceptable components. See Remington's Pharmaceutical Science (15th ed., Mack Publishing Company, Easton, Pennsylvania (1980)). The desired form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents include, but are not limited to, distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, microcrystalline cellulose, corn starch, sodium starch glycolate, and alginic acid. Tablet binders that can be used include acacia, methylcellulose, sodium carboxymethylcellulose, polyvinylpyrrolidone (Povidone™), hydroxypropyl methylcellulose, sucrose, starch, and ethylcellulose. Lubricants that can be used include magnesium stearates, stearic acid, silicone fluid, talc, waxes, oils, and colloidal silica. Additional excipients include, for example, colorants, taste-masking agents, solubility aids, suspension agents, compressing agents, enteric coatings, sustained release aids, and the like.

In some embodiments, the compositions can be administered in the form of a depot injection or implant preparation, which can be formulated in such a manner as to permit a sustained release. An exemplary composition comprises any one or more of the compositions described herein formulated in aqueous buffer.

In some embodiments, liquid formulations of a pharmaceutical composition for oral administration prepared in water or other aqueous vehicles can contain various suspending agents such as methylcellulose, alginates, tragacanth, pectin, kelgin, carrageenan, acacia, polyvinylpyrrolidone, and polyvinyl alcohol. Liquid formulations of pharmaceutical compositions can also include solutions, emulsions, syrups and elixirs containing, together with the active compound(s), wetting agents, sweeteners, and coloring and flavoring agents. Various liquid and powder formulations of the pharmaceutical compositions can be prepared by conventional methods for inhalation into the lungs of the mammal to be treated.

In some embodiments, liquid formulations of a pharmaceutical composition for injection can comprise various carriers such as vegetable oils, dimethylacetamide, dimethylformamide, ethyl lactate, ethyl carbonate, isopropyl myristate, ethanol, polyols such as, for example, glycerol, propylene glycol, liquid polyethylene glycol, and the like. In some embodiments, the composition includes a citrate/sucrose/tween carrier. For intravenous injections, water soluble versions of the compositions can be administered by the drip method, whereby a pharmaceutical formulation containing the antifungal agent and a physiologically acceptable excipient is infused. Physiologically acceptable excipients can include, for example, 5% dextrose, 0.9% saline, Ringer's solution or other suitable excipients. A suitable insoluble form of the composition can be prepared and administered as a suspension in an aqueous base or a pharmaceutically acceptable oil base, such as an ester of a long chain fatty acid such as, for example, ethyl oleate.

The compositions can be, for example, injectable solutions, aqueous suspensions or solutions, non-aqueous suspensions or solutions, solid and liquid oral formulations, salves, gels, ointments, intradermal patches, creams, aerosols, lotions, tablets, capsules, sustained release formulations, and the like. In some embodiments, for topical applications, the pharmaceutical compositions can be formulated in a suitable ointment. In some embodiments, a topical semi-solid ointment formulation typically comprises a concentration of the active ingredient from about 1 to 20%, or from 5 to 10%, in a carrier, such as a pharmaceutical cream base. Some examples of formulations of a composition for topical use include, but are not limited to, drops, tinctures, lotions, creams, solutions, and ointments containing the active ingredient and various supports and vehicles.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or microparticles such as polylactide, polyglycolide, or copolymer for enhanced adjuvant effect (see Langer, Science, 1990, 249, 1527 and Hanes, Advanced Drug Delivery Reviews, 1997, 28, 97). A sterile injectable preparation such as, for example, a sterile injectable aqueous or oleaginous suspension can also be prepared. This suspension may be formulated according to techniques known in the art using suitable dispersing, wetting, and suspending agents. In some embodiments, the pharmaceutical composition can be delivered in a microencapsulation device so as to reduce or prevent a host immune response against the protein.

In some embodiments, any of the Mtb antigens, constructs, vectors, or cells described herein, or compositions comprising the same, can be combined into a single therapeutic or prophylactic regimen. For example, in some embodiments, an antigen matched BCG can be combined or used with a recombinant protein vaccine.

In some embodiments, any of the Mtb antigens, constructs, vectors, or cells described herein, or compositions comprising the same, can be administered to a mammal as an aerosol. In some embodiments, the aerosol inocula comprises saline. Conventional aerosol delivery devices include, but are not limited to, a pressurized metered dose inhaler (pMDI) and a dry power inhaler (DPI), both of which deliver a dry powder formulation, and nebulizers such as the PARI eFlow device, which delivers an aqueous dose as a fine mist. In some embodiments, the aerosol delivery device is a Pari eFlow portable electronic aerosol delivery platform attached to a delivery mask. In some embodiments, the average particle size is from about 1 μm to about 10 μm, from about 1 μm to about 5 μm, from about 3 μm to about 5 μm, from about 4 μm to about 5 μm, or from about 3.9 μm to about 4.9 μm. In some embodiments, the aerosol is in a volume from about 0.1 ml to about 5 ml, from about 0.1 ml to about 2 ml, from about 0.1 ml to about 1.5 ml, from about 0.5 ml to about 1.5 ml, from about 0.5 ml to about 1.2 ml, from about 0.7 ml to about 1.2 ml, or about 1 ml.

Effective doses of the compositions of the present disclosure, for the treatment of a condition vary depending upon many different factors, including means of administration, target site, physiological state of the subject, whether the subject is human or an animal, other medications administered, and whether treatment is prophylactic or therapeutic. Usually, the subject is a human but non-human mammals including transgenic mammals can also be treated.

In some embodiments, the compositions can be administered to a subject by injection intravenously, subcutaneously, intraperitoneally, intramuscularly, intramedullarily, intraventricularly, intraepidurally, intraarterially, intravascularly, intraarticularly, intrasynovially, intrasternally, intrathecally, intrahepatically, intraspinally, intratumorly, intracranially, enteral, intrapulmonary, transmucosal, intrauterine, sublingual, or locally at sites of inflammation or tumor growth by using standard methods. In some embodiments, the compositions can be administered to a subject by injection intravenously. Alternately, the compositions can be administered to a subject by routes including oral, nasal, ophthalmic, rectal, or topical. The most typical route of administration is intravascular, subcutaneous, or intramuscular, although other routes can be effective. In some embodiments, compositions are administered as a sustained release composition or device, such as a Medipad™ device. The composition can also be administered via the respiratory tract, for example, using a dry powder inhalation device, nebulizer, or a metered dose inhaler. The composition can also be administered by traditional syringes, needleless injection devices, “microprojectile bombardment gone guns,” or other physical methods such as electroporation (“EP”), “hydrodynamic method”, or ultrasound.

In some embodiments, the composition can be administered to a subject by sustained release administration, by such means as depot injections of erodible implants directly applied during surgery or by implantation of an infusion pump or a biocompatible sustained release implant into the subject. Alternately, the composition can be administered to a subject by injectable depot routes of administration, such as by using 1-, 3-, or 6-month depot injectable or biodegradable materials and methods, or by applying to the skin of the subject a transdermal patch containing the composition, and leaving the patch in contact with the subject's skin, generally for 1 to 5 hours per patch.

In some embodiments, the compositions comprise about 1 nanogram to about 10 mg of nucleic acid. In some embodiments, the compositions comprise: 1) at least 10, 15, 20, 25, 30, 35, 40, 45, 50, 55, 60, 65, 70, 75, 80, 85, 90, 95 or 100 nanograms, or at least 1, 5, 10, 15, 20, 25, 30, 35, 40, 45, 50, 55, 60, 65, 70, 75, 80, 85, 90, 95,100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995 or 1000 micrograms, or at least 1.5, 2, 2.5, 3, 3.5, 4, 4.5, 5, 5.5, 6, 6.5, 7, 7.5, 8, 8.5, 9, 9.5 or 10 mg or more; and 2) up to and including 15, 20, 25, 30, 35, 40, 45, 50, 55, 60, 65, 70, 75, 80, 85, 90, 95 or 100 nanograms, or up to and including 1, 5, 10, 15, 20, 25, 30, 35, 40, 45, 50, 55, 60, 65, 70, 75, 80, 85, 90, 95,100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995, or 1000 micrograms, or up to and including 1.5, 2, 2.5, 3, 3.5, 4, 4.5, 5, 5.5, 6, 6.5, 7, 7.5, 8, 8.5, 9, 9.5 or 10 mg.

In some embodiments, the compositions comprise about 5 ng to about 10 mg of nucleic acid molecule. In some embodiments, the compositions comprise about 25 ng to about 5 mg of nucleic acid molecule. In some embodiments, the compositions contain about 50 ng to about 1 mg of nucleic acid molecule. In some embodiments, the compositions contain about 0.1 to about 500 μg of nucleic acid molecule. In some embodiments, the compositions contain about 1 μg to about 350 μg of nucleic acid molecule. In some embodiments, the compositions contain about 5 μg to about 250 μg of nucleic acid molecule. In some embodiments, the compositions contain about 10 μg to about 200 μg of nucleic acid molecule. In some embodiments, the compositions contain about 15 μg to about 150 μg of nucleic acid molecule. In some embodiments, the compositions contain about 20 μg to about 100 μg of nucleic acid molecule. In some embodiments, the compositions contain about 25 μg to about 75 μg of nucleic acid molecule. In some embodiments, the compositions contain about 30 μg to about 50 μg of nucleic acid molecule. In some embodiments, the compositions contain about 35 μg to about 40 μg of nucleic acid molecule. In some embodiments, the compositions contain about 100 μg to about 200 μg of nucleic acid molecule. In some embodiments, the compositions comprise about 10 μg to about 100 μg of nucleic acid molecule. In some embodiments, the compositions comprise about 20 μg to about 80 μg of nucleic acid molecule. In some embodiments, the compositions comprise about 25 μg to about 60 μg of nucleic acid molecule. In some embodiments, the compositions comprise about 30 ng to about 50 μg of nucleic acid molecule. In some embodiments, the compositions comprise about 35 ng to about 45 μg of nucleic acid molecule. In some embodiments, the compositions contain about 0.1 μg to about 500 μg of nucleic acid molecule.

In some embodiments, the compositions contain about 1 μg to about 350 μg of nucleic acid molecule. In some embodiments, the compositions contain about 25 μg to about 250 μg of nucleic acid molecule. In some embodiments, the compositions contain about 100 μg to about 200 μg of nucleic acid molecule.

In some embodiments, the delivery platforms described herein can be used either in a single administration alone or in combinations as matched antigen prime-boost approaches. In addition, the use of these antigens in a single vector system, which is envisioned to be used as an antigen matched prime for a boost with any of the modalities above, including protein, viral vectors, nucleic acids, or others. For example, the same Mtb antigen construct can be used as both the prime and the boost. In other embodiments, a first Mtb antigen construct can be used as the prime and a second different Mtb antigen construct can be used as the boost (i.e., heterologous prime-boost). In some embodiments, the prime is a DNA or RNA (such as mRNA) prime and the boost is a viral vector boost. In some embodiments, the prime is a viral vector prime and the boost is a DNA or RNA (such as mRNA) boost.

The compositions can be formulated according to the mode of administration to be used. In cases where compositions are injectable pharmaceutical compositions, they are sterile, pyrogen free and particulate free. An isotonic formulation can be used. Generally, additives for isotonicity can include sodium chloride, dextrose, mannitol, sorbitol and lactose. In some cases, isotonic solutions such as phosphate buffered saline are suitable. Stabilizers include gelatin and albumin. In some embodiments, a vasoconstriction agent is added to the formulation.

The compositions can further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freund's incomplete adjuvant, LPS analog including monophosphoryl lipid A, muramyl peptides, quinone analogs, vesicles such as squalene and squalane, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more suitably, the poly-L-glutamate is present in the composition at a concentration less than 6 mg/ml. The transfection facilitating agent can also include surface active agents such as immune-stimulating complexes (ISCOMS), Freunds incomplete adjuvant, LPS analog including monophosphoryl lipid A, muramyl peptides, quinone analogs and vesicles such as squalene and squalane, and hyaluronic acid can also be used administered in conjunction with the genetic construct. In some embodiments, the plasmid compositions can also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. In some embodiments, the transfection facilitating agent is a polyanion, polycation, including poly-L-glutamate (LGS), or lipid. Concentration of the transfection agent in the composition is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient may be an adjuvant. The adjuvant may be other genes that are expressed in alternative plasmid or are delivered as proteins in combination with the plasmid above. The adjuvant may be selected from the group consisting of: α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant may be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes which may be useful adjuvants include those encoding: MCP-1, MIP-1a, MIP-1p, IL-8, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFKB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The present disclosure also provides kits comprising any of the Mtb antigens, fragments thereof, fusion proteins, nucleic acid molecules, vectors, or cells, described herein. The kit can include, for example, container(s), package(s) or dispenser(s) along with labels and instructions for administration or use.

The present disclosure also provides methods of eliciting an immune response against Mycobacterium tuberculosis in a mammal comprising administering to the mammal an immunologically sufficient amount of one or more fusion proteins described herein. Any of the fusion proteins described herein can be administered. In some embodiments, the fusion protein comprises Rv3872-Rv1788-Rv3893c-Rv0285-Rv1818c-Rv0159c-Rv1172c (Construct A; see Table 2). In some embodiments, the fusion protein comprises Rv3873-Rv1387-Rv3892c (Construct B; see Table 4). In some embodiments, the fusion protein comprises Rv1789-Rv1800-Rv1039c (Construct C; see Table 4). In some embodiments, the fusion protein comprises Rv3017c-Rv3020c-Rv3019c-Rv3891c-Rv2346c-Rv3445c (Construct D; see Table 6). In some embodiments, the fusion protein comprises Rv3619c-Rv3875-Rv3874 (Construct E; see Table 6). In some embodiments, the fusion protein comprises Rv2719c-Rv0010c-Rv1872c (Construct F; see Table 8). In some embodiments, the fusion protein comprises Rv0012-Rv0990c-Rv0995 (Construct G; see Table 8).

The present disclosure also provides methods of eliciting an immune response against Mycobacterium tuberculosis in a mammal comprising administering to the mammal an immunologically sufficient amount of any of the Mtb compositions described herein. Any of the compositions comprising two or more Mtb antigens can be administered. In some embodiments, the composition comprises Rv3872, Rv1788, Rv3893c, Rv0285, Rv1818c, Rv0159c, and Rv1172c. In some embodiments, the composition comprises Rv3873, Rv1387, and Rv3892c. In some embodiments, the composition comprises Rv1789, Rv1800, and Rv1039c. In some embodiments, the composition comprises Rv3017c, Rv3020c, Rv3019c, Rv3891c, Rv2346c, and Rv3445c. In some embodiments, the composition comprises Rv3619c, Rv3875, and Rv3874. In some embodiments, the composition comprises Rv2719c, Rv0010c, and Rv1872c. In some embodiments, the composition comprises Rv0012, Rv0990c, and Rv0995.

The present disclosure also provides methods of eliciting an immune response against Mycobacterium tuberculosis in a mammal comprising administering to the mammal an immunologically sufficient amount of a composition comprising at least two or three Mtb antigens, and a pharmaceutically acceptable carrier; wherein the composition comprises at least one nucleic acid molecule encoding at least one of the Mtb antigens. Any of the compositions comprising a mixture of one or more Mtb antigen proteins and one of more nucleic acid molecules encoding one or more Mtb antigens described herein can be administered.

The fusion proteins and compositions described herein can be used to treat or prevent tuberculosis. In some embodiments, the method comprises administering to a human a therapeutically- or prophylactically-effective amount of any of the fusion proteins or compositions described herein such that the tuberculosis infection is diminished or prevented.

In some embodiments, the subject being treated will have been previously diagnosed as having tuberculosis. Such subjects will, thus, have been diagnosed as being in need of such treatment. Alternately, the treatment may be intended to prevent a tuberculosis infection in a subject that does not yet have tuberculosis or to a subject that is travelling to an area where tuberculosis is prevalent.

Treatment of a subject suffering from tuberculosis can be monitored using standard methods. Some methods entail determining a baseline value, for example, of an antibody level or profile in a subject, before administering a dosage of agent, and comparing this with a value for the profile or level after treatment. A significant increase such as, for example, greater than the typical margin of experimental error in repeat measurements of the same sample, expressed as one standard deviation from the mean of such measurements in value of the level or profile signals a positive treatment outcome (i.e., that administration of the agent has achieved a desired response). If the value for immune response does not change significantly, or decreases, a negative treatment outcome is indicated.

In some embodiments, a control value such as a mean and standard deviation, of level or profile is determined for a control population. Typically the individuals in the control population have not received prior treatment. Measured values of the level or profile in a subject after administering a therapeutic agent are then compared with the control value. A significant increase relative to the control value, such as greater than one standard deviation from the mean, signals a positive or sufficient treatment outcome. A lack of significant increase or a decrease signals a negative or insufficient treatment outcome. Administration of the therapeutic is generally continued while the level is increasing relative to the control value. As before, attainment of a plateau relative to control values is an indicator that the administration of treatment can be discontinued or reduced in dosage and/or frequency.

In other embodiments, a control value of the level or profile, such as a mean and standard deviation, is determined from a control population of individuals who have undergone treatment with a therapeutic agent and whose levels or profiles have plateaued in response to treatment. Measured values of levels or profiles in a subject are compared with the control value. If the measured level in a subject is not significantly different, such as by more than one standard deviation, from the control value, treatment can be discontinued. If the level in a subject is significantly below the control value, continued administration of agent is warranted. If the level in the subject persists below the control value, then a change in treatment may be indicated.

In some embodiments, a subject who is not presently receiving treatment but has undergone a previous course of treatment is monitored for antibody levels or profiles to determine whether a resumption of treatment is required. The measured level or profile in the subject can be compared with a value previously achieved in the subject after a previous course of treatment. A significant decrease relative to the previous measurement, such as greater than a typical margin of error in repeat measurements of the same sample, is an indication that treatment can be resumed. Alternately, the value measured in a subject can be compared with a control value (mean plus standard deviation) determined in a population of subjects after undergoing a course of treatment. Alternately, the measured value in a subject can be compared with a control value in populations of prophylactically treated subjects who remain free of symptoms of disease, or populations of therapeutically treated subjects who show amelioration of disease characteristics. In all of these cases, a significant decrease relative to the control level, such as more than a standard deviation, is an indicator that treatment should be resumed in a subject.

In some methods, a baseline measurement of antibody to a given antigen in the subject is made before administration, a second measurement is made soon thereafter to determine the peak antibody level, and one or more further measurements are made at intervals to monitor decay of antibody levels. When the level of antibody has declined to baseline or a predetermined percentage of the peak less baseline, such as 50%, 25% or 10%, administration of a further dosage of antigen is administered. In some embodiments, peak or subsequent measured levels less background are compared with reference levels previously determined to constitute a beneficial prophylactic or therapeutic treatment regime in other subjects. If the measured antibody level is significantly less than a reference level, such as less than the mean minus one standard deviation of the reference value in population of subjects benefiting from treatment, administration of an additional dosage of antigen is indicated.

In some embodiments, the subject(s) that can be treated by the above-described methods is an animal, including mammals and non-mammals. Suitable mammals, include, but are not limited to, humans, non-human primates, rodents (including rats, mice, hamsters and guinea pigs) cow, horse, sheep, badger, opossum, goat, pig, dog and cat. In most instances, the mammal is a human. In some embodiments, the non-mammal is a fish. Immunization of animals with any one or more of the vaccines described herein can prevent zoonotic transmission (i.e., transition of a disease, such as TB, from an animal to a human).

The present disclosure also provides fusion proteins for use in the preparation of a medicament for treating or preventing a Mycobacterium tuberculosis infection. Any of the fusion proteins described herein can be administered. In some embodiments, the fusion protein comprises Rv3872-Rv1788-Rv3893c-Rv0285-Rv1818c-Rv0159c-Rv1172c (Construct A; see Table 2). In some embodiments, the fusion protein comprises Rv3873-Rv1387-Rv3892c (Construct B; see Table 4). In some embodiments, the fusion protein comprises Rv1789-Rv1800-Rv1039c (Construct C; see Table 4). In some embodiments, the fusion protein comprises Rv3017c-Rv3020c-Rv3019c-Rv3891c-Rv2346c-Rv3445c (Construct D; see Table 6). In some embodiments, the fusion protein comprises Rv3619c-Rv3875-Rv3874 (Construct E; see Table 6). In some embodiments, the fusion protein comprises Rv2719c-Rv0010c-Rv1872c (Construct F; see Table 8). In some embodiments, the fusion protein comprises Rv0012-Rv0990c-Rv0995 (Construct G; see Table 8).

The present disclosure also provides fusion proteins for use in treating or preventing a Mycobacterium tuberculosis infection. Any of the fusion proteins described herein can be administered. In some embodiments, the fusion protein comprises Rv3872-Rv1788-Rv3893c-Rv0285-Rv1818c-Rv0159c-Rv1172c (Construct A; see Table 2). In some embodiments, the fusion protein comprises Rv3873-Rv1387-Rv3892c (Construct B; see Table 4). In some embodiments, the fusion protein comprises Rv1789-Rv1800-Rv1039c (Construct C; see Table 4). In some embodiments, the fusion protein comprises Rv3017c-Rv3020c-Rv3019c-Rv3891c-Rv2346c-Rv3445c (Construct D; see Table 6). In some embodiments, the fusion protein comprises Rv3619c-Rv3875-Rv3874 (Construct E; see Table 6). In some embodiments, the fusion protein comprises Rv2719c-Rv0010c-Rv1872c (Construct F; see Table 8). In some embodiments, the fusion protein comprises Rv0012-Rv0990c-Rv0995 (Construct G; see Table 8).

The present disclosure also provides use of a fusion protein in the preparation of a medicament for treating or preventing a Mycobacterium tuberculosis infection. Any of the fusion proteins described herein can be administered. In some embodiments, the fusion protein comprises Rv3872-Rv1788-Rv3893c-Rv0285-Rv1818c-Rv0159c-Rv1172c (Construct A; see Table 2). In some embodiments, the fusion protein comprises Rv3873-Rv1387-Rv3892c (Construct B; see Table 4). In some embodiments, the fusion protein comprises Rv1789-Rv1800-Rv1039c (Construct C; see Table 4). In some embodiments, the fusion protein comprises Rv3017c-Rv3020c-Rv3019c-Rv3891c-Rv2346c-Rv3445c (Construct D; see Table 6). In some embodiments, the fusion protein comprises Rv3619c-Rv3875-Rv3874 (Construct E; see Table 6). In some embodiments, the fusion protein comprises Rv2719c-Rv0010c-Rv1872c (Construct F; see Table 8). In some embodiments, the fusion protein comprises Rv0012-Rv0990c-Rv0995 (Construct G; see Table 8).

The present disclosure also provides uses of a fusion protein in treating or preventing a Mycobacterium tuberculosis infection. Any of the fusion proteins described herein can be administered. In some embodiments, the fusion protein comprises Rv3872-Rv1788-Rv3893c-Rv0285-Rv1818c-Rv0159c-Rv1172c (Construct A; see Table 2). In some embodiments, the fusion protein comprises Rv3873-Rv1387-Rv3892c (Construct B; see Table 4). In some embodiments, the fusion protein comprises Rv1789-Rv1800-Rv1039c (Construct C; see Table 4). In some embodiments, the fusion protein comprises Rv3017c-Rv3020c-Rv3019c-Rv3891c-Rv2346c-Rv3445c (Construct D; see Table 6). In some embodiments, the fusion protein comprises Rv3619c-Rv3875-Rv3874 (Construct E; see Table 6). In some embodiments, the fusion protein comprises Rv2719c-Rv0010c-Rv1872c (Construct F; see Table 8). In some embodiments, the fusion protein comprises Rv0012-Rv0990c-Rv0995 (Construct G; see Table 8).

The present disclosure also provides compositions for use in the preparation of a medicament for treating or preventing a Mycobacterium tuberculosis infection. Any of the compositions comprising two or more Mtb antigens can be administered. In some embodiments, the composition comprises Rv3872, Rv1788, Rv3893c, Rv0285, Rv1818c, Rv0159c, and Rv1172c. In some embodiments, the composition comprises Rv3873, Rv1387, and Rv3892c. In some embodiments, the composition comprises Rv1789, Rv1800, and Rv1039c. In some embodiments, the composition comprises Rv3017c, Rv3020c, Rv3019c, Rv3891c, Rv2346c, and Rv3445c. In some embodiments, the composition comprises Rv3619c, Rv3875, and Rv3874. In some embodiments, the composition comprises Rv2719c, Rv0010c, and Rv1872c. In some embodiments, the composition comprises Rv0012, Rv0990c, and Rv0995.

The present disclosure also provides compositions for use in treating or preventing a Mycobacterium tuberculosis infection. Any of the compositions comprising two or more Mtb antigens can be administered. In some embodiments, the composition comprises Rv3872, Rv1788, Rv3893c, Rv0285, Rv1818c, Rv0159c, and Rv1172c. In some embodiments, the composition comprises Rv3873, Rv1387, and Rv3892c. In some embodiments, the composition comprises Rv1789, Rv1800, and Rv1039c. In some embodiments, the composition comprises Rv3017c, Rv3020c, Rv3019c, Rv3891c, Rv2346c, and Rv3445c. In some embodiments, the composition comprises Rv3619c, Rv3875, and Rv3874. In some embodiments, the composition comprises Rv2719c, Rv0010c, and Rv1872c. In some embodiments, the composition comprises Rv0012, Rv0990c, and Rv0995.

The present disclosure also provides uses of a composition in the preparation of a medicament for treating or preventing a Mycobacterium tuberculosis infection. Any of the compositions comprising two or more Mtb antigens can be administered. In some embodiments, the composition comprises Rv3872, Rv1788, Rv3893c, Rv0285, Rv1818c, Rv0159c, and Rv1172c. In some embodiments, the composition comprises Rv3873, Rv1387, and Rv3892c. In some embodiments, the composition comprises Rv1789, Rv1800, and Rv1039c. In some embodiments, the composition comprises Rv3017c, Rv3020c, Rv3019c, Rv3891c, Rv2346c, and Rv3445c. In some embodiments, the composition comprises Rv3619c, Rv3875, and Rv3874. In some embodiments, the composition comprises Rv2719c, Rv0010c, and Rv1872c. In some embodiments, the composition comprises Rv0012, Rv0990c, and Rv0995.

The present disclosure also provides uses of a composition in treating or preventing a Mycobacterium tuberculosis infection. Any of the compositions comprising two or more Mtb antigens can be administered. In some embodiments, the composition comprises Rv3872, Rv1788, Rv3893c, Rv0285, Rv1818c, Rv0159c, and Rv1172c. In some embodiments, the composition comprises Rv3873, Rv1387, and Rv3892c. In some embodiments, the composition comprises Rv1789, Rv1800, and Rv1039c. In some embodiments, the composition comprises Rv3017c, Rv3020c, Rv3019c, Rv3891c, Rv2346c, and Rv3445c. In some embodiments, the composition comprises Rv3619c, Rv3875, and Rv3874. In some embodiments, the composition comprises Rv2719c, Rv0010c, and Rv1872c. In some embodiments, the composition comprises Rv0012, Rv0990c, and Rv0995.

The present disclosure also provides compositions for use in the preparation of a medicament for treating or preventing a Mycobacterium tuberculosis infection, wherein the composition comprises at least two or three Mtb antigens, and a pharmaceutically acceptable carrier, wherein the composition comprises at least one nucleic acid molecule encoding at least one of the Mtb antigens.

The present disclosure also provides compositions for use in treating or preventing a Mycobacterium tuberculosis infection, wherein the composition comprises at least two or three Mtb antigens, and a pharmaceutically acceptable carrier, wherein the composition comprises at least one nucleic acid molecule encoding at least one of the Mtb antigens.

The present disclosure also provides uses of a composition in the preparation of a medicament for treating or preventing a Mycobacterium tuberculosis infection, wherein the composition comprises at least two or three Mtb antigens, and a pharmaceutically acceptable carrier, wherein the composition comprises at least one nucleic acid molecule encoding at least one of the Mtb antigens.

The present disclosure also provides uses of a composition in treating or preventing a Mycobacterium tuberculosis infection, wherein the composition comprises at least two or three Mtb antigens, and a pharmaceutically acceptable carrier, wherein the composition comprises at least one nucleic acid molecule encoding at least one of the Mtb antigens.

The present disclosure also provides any of the fusion proteins described herein, or any of the compositions described herein, or any of the cells described herein, or any of the vectors described herein, or any of the methods described herein, or any of the uses described herein, substantially as described herein.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A vector encoding a fusion protein comprising at least two Mth PE antigens chosen from Rv3872, Rv1788, Rv3893c, Rv0285, Rv1818c, Rv0159c, and Rv1172c;
at least two Mtb PPE antigens chosen from Rv3873, Rv1387, Rv3892c, Rv1789, Rv1800, and Rv1039c;
at least two Mtb ESX antigens chosen from RV3017c, Rv3020c, Rv3019c, Rv3891c, Rv2346c, Rv3445c, Rv3619c, Rv3875, and Rv3874; or
at least two Mtb variable antigens chosen from Rv2719c, Rv0010c, Rv1872c, Rv0012, Rv0990c, and Rv0995.

2. The vector according to claim 1 wherein: Rv3872 SEQ ID NO:2, Rv1788 comprises SEQ ID NO:4, Rv3893c comprises SEQ ID NO:6, Rv0285 comprises SEQ ID NO:8, Rv1818c comprises SEQ ID NO:10, Rv0159c comprises SEQ ID NO:12, Rv1172c comprises SEQ ID NO:14, Rv1387 comprises SEQ ID NO:21, Rv3892c comprises SEQ ID NO:23, Rv1789 comprises SEQ ID NO:25, Rv1800 comprises SEQ ID NO:27, Rv1039c comprises SEQ ID NO:29, RV3017c comprises SEQ ID NO:35, Rv3020c comprises SEQ ID NO:37, Rv3019c comprises SEQ ID NO:39, Rv3891c comprises SEQ ID NO:41, Rv2346c comprises SEQ ID NO:43, Rv3445c comprises SEQ ID NO:45, Rv3619c comprises SEQ ID NO:47, Rv3875 comprises SEQ ID NO:49, Rv3874 comprises SEQ ID NO:51, Rv2719c comprises SEQ ID NO:57, Rv0010c comprises SEQ ID NO:59, Rv1872c comprises SEQ ID NO:61, Rv0012 comprises SEQ ID NO:63, Rv0990c comprises SEQ ID NO:65, or Rv0995 comprises SEQ ID NO:67.

3. The vector according to claim 1 wherein: Rv3872 is encoded by a nucleotide sequence set forth in SEQ ID NO:1, Rv1788 is encoded by a nucleotide sequence set forth in SEQ ID NO:3, Rv3893c is encoded by a nucleotide sequence set forth in SEQ ID NO:5, Rv0285 is encoded by a nucleotide sequence set forth in SEQ ID NO:7, Rv1818c is encoded by a nucleotide sequence set forth in SEQ ID NO:9, Rv0159c is encoded by a nucleotide sequence set forth in SEQ ID NO:11, Rv1172c is encoded by a nucleotide sequence set forth in SEQ ID NO:13, Rv1387 is encoded by a nucleotide sequence set forth in SEQ ID NO:20, Rv3892c is encoded by a nucleotide sequence set forth in SEQ ID NO:22, Rv1789 is encoded by a nucleotide sequence set forth in SEQ ID NO:24, Rv1800 is encoded by a nucleotide sequence set forth in SEQ ID NO:26, Rv1039c is encoded by a nucleotide sequence set forth in SEQ ID NO:28, RV3017c is encoded by a nucleotide sequence set forth in SEQ ID NO:34, Rv3020c is encoded by a nucleotide sequence set forth in SEQ ID NO:36, Rv3019c is encoded by a nucleotide sequence set forth in SEQ ID NO:38, Rv3891c is encoded by a nucleotide sequence set forth in SEQ ID NO:40, Rv2346c is encoded by a nucleotide sequence set forth in SEQ ID NO:42, Rv3445c is encoded by a nucleotide sequence set forth in SEQ ID NO:44, Rv3619c is encoded by a nucleotide sequence set forth in SEQ ID NO:46, Rv3875 is encoded by a nucleotide sequence set forth in SEQ ID NO:48, Rv3874 is encoded by a nucleotide sequence set forth in SEQ ID NO:50, Rv2719c is encoded by a nucleotide sequence set forth in SEQ ID NO:56, Rv0010c is encoded by a nucleotide sequence set forth in SEQ ID NO:58, Rv1872c is encoded by a nucleotide sequence set forth in SEQ ID NO:60, Rv0012 is encoded by a nucleotide sequence set forth in SEQ ID NO:62, Rv0990c is encoded by a nucleotide sequence set forth in SEQ ID NO:64, or Rv0995 is encoded by a nucleotide sequence set forth in SEQ ID NO:66.

4-20. (canceled).

21. The vector according to claim 1, wherein the fusion protein comprises Rv3872 and 10 Rv1788; Rv3893c, Rv0285, and Rv1818c; or Rv0159c and Rv1172c.

22-23. (canceled).

24. The vector according to claim 1, wherein the fusion protein comprises Rv3872-Rv1788-Rv3893c-Rv0285-Rv1818c-Rv0159c-Rv1172c.

25-26. (canceled).

27. The vector according to claim 24, wherein Rv3872-Rv1788-Rv3893c-Rv0285-Rv1818c-Rv0159c-Rv1172c comprises the amino acid sequence set forth in SEQ ID NO:16.

28-45. (canceled).

46. The vector according to claim 1 wherein the fusion protein comprises Rv3873, Rv1387, and Rv3892c; or Rv1789, Rv1800, and Rv1039c.

47-48. (canceled).

49. The vector according to claim 29 wherein the fusion protein comprises Rv3873-Rv1387-Rv3892c.

50-58. (canceled).

59. The vector of claim 1, wherein the fusion protein comprises at least two Mtb ESX antigens chosen from 15 Rv3017c, Rv3020c, Rv3019c, Rv3891c, Rv2346c, Rv3445c, Rv3619c, Rv3875, and Rv3874.

60-84. (canceled).

85. The vector according to claim 59 wherein the fusion protein comprises Rv3017c, Rv3020c, Rv3019c, Rv3891c, Rv2346c, and Rv3445c; or Rv3619c, Rv3875, and Rv3874.

86-87. (canceled).

88. The vector according to claim 85 wherein the fusion protein comprises Rv3017c-Rv3020c-Rv3019c-Rv3 891 c-Rv2346c-Rv3445c.

89-92. (canceled).

93. The vector according to claim 85 wherein the fusion protein comprises Rv3619c-Rv3875-Rv3874.

94-114. (canceled).

115. The vector according to claim 1 wherein the fusion protein comprises Rv2719c, Rv0010c, and Rv1872c; or Rv0012, Rv0990c, and Rv0995.

116-117. (canceled).

118. The vector according to claim 115 wherein the fusion protein comprises Rv2719c-Rv0010c-Rv1872c.

119-122. (canceled).

123. The vector according to claim 115 wherein the fusion protein comprises Rv0012-Rv0990c-Rv0995.

124-127. (canceled).

128. A pharmaceutical composition comprising the vector of claim 1 and a pharmaceutically acceptable carrier.

129. (canceled).

130. The vector according to claim 1 which is a viral vector.

131-143. (canceled).

144. A method of eliciting an immune response against Mycobacterium tuberculosis in a mammal comprising administering to the mammal an immunologically sufficient amount of the vector according to claim 1.

145. (canceled).

146. A kit comprising the vector of claim 1.

147. A method of eliciting an immune response against Mycobacterium tuberculosis in a mammal comprising administering the vector according to claim 1.


SEQUENCE LISTING (71 sequences)

SEQ ID NO: 1 - DNA, 300 bp, synthetic construct
  misc_feature 1..300: note = Rv3872 (PE35)
  source 1..300: mol_type = other DNA
atggagaagatgagccacgaccccatcgccgccgacatcggcacccaggtgagcgacaacgccctgcacggcgtgaccgccggcagcaccgccctgaccagcgtgaccggcctggtgcccgccggcgccgacgaggtgagcgcccaggccgccaccgccttcaccagcgagggcatccagctgctggccagcaacgccagcgcccaggaccagctgcacagggccggcgaggccgtgcaggacgtggccaggacctacagccagatcgacgacggcgccgccggcgtgttcgccgagtga

SEQ ID NO: 2 - protein, 99 aa, synthetic construct
  REGION 1..99: note = Rv3872 (PE35)
MEKMSHDPIAADIGTQVSDNALHGVTAGSTALTSVTGLVPAGADEVSAQAATAFTSEGIQLLASNASAQDQLHRAGEAVQDVARTYSQIDDGAAGVFAE

SEQ ID NO: 3 - DNA, 300 bp, synthetic construct
  misc_feature 1..300: note = Rv1788 (PE18)
  source 1..300: mol_type = other DNA
atgagcttcgtgaccacccagcccgaggccctggccgccgccgccggcagcctgcagggcatcggcagcgccctgaacgcccagaacgccgccgccgccacccccaccaccggcgtggtgcccgccgccgccgacgaggtgagcgccctgaccgccgcccagttcgccgcccacgcccagatctaccaggccgtgagcgcccaggccgccgccatccacgagatgttcgtgaacaccctgcagatgagcagcggcagctacgccgccaccgaggccgccaacgccgccgccgccggctga

SEQ ID NO: 4 - protein, 99 aa, synthetic construct
  REGION 1..99: note = Rv1788 (PE18)
MSFVTTQPEALAAAAGSLQGIGSALNAQNAAAATPTTGVVPAAADEVSALTAAQFAAHAQIYQAVSAQAAAIHEMFVNTLQMSSGSYAATEAANAAAAG

SEQ ID NO: 5 - DNA, 234 bp, synthetic construct
  misc_feature 1..234: note = Rv3893c (PE36)
  source 1..234: mol_type = other DNA
atggtgtggagcgtgcagcccgaggccgtgctggccagcgccgccgccgagagcgccatcagcgccgagaccgaggccgccgccgccggcgccgcccccgccctgctgagcaccacccccatgggcggcgaccccgacagcgccatgttcagcgccgccctgaacgcctgcggcgccagctacctgggcgtggtggccgagcacgccagccagaggggcctgttcgccggctga

SEQ ID NO: 6 - protein, 77 aa, synthetic construct
  REGION 1..77: note = Rv3893c (PE36)
MVWSVQPEAVLASAAAESAISAETEAAAAGAAPALLSTTPMGGDPDSAMFSAALNACGASYLGVVAEHASQRGLFAG

SEQ ID NO: 7 - DNA, 309 bp, synthetic construct
  misc_feature 1..309: note = Rv0285 (PE5)
  source 1..309: mol_type = other DNA
atgaccctgagggtggtgcccgagggcctggccgccgccagcgccgccgtggaggccctgaccgccaggctggccgccgcccacgccagcgccgcccccgtgatcaccgccgtggtgccccccgccgccgaccccgtgagcctgcagaccgccgccggcttcagcgcccagggcgtggagcacgccgtggtgaccgccgagggcgtggaggagctgggcagggccggcgtgggcgtgggcgagagcggcgccagctacctggccggcgacgccgccgccgccgccacctacggcgtggtgggcggctga

SEQ ID NO: 8 - protein, 102 aa, synthetic construct
  REGION 1..102: note = Rv0285 (PE5)
MTLRVVPEGLAAASAAVEALTARLAAAHASAAPVITAVVPPAADPVSLQTAAGFSAQGVEHAVVTAEGVEELGRAGVGVGESGASYLAGDAAAAATYGVVGG

SEQ ID NO: 9 - DNA, 303 bp, synthetic construct
  misc_feature 1..303: note = Rv1818c (PE_PGRS33)
  source 1..303: mol_type = other DNA
atgagcttcgtggtgaccatccccgaggccctggccgccgtggccaccgacctggccggcatcggcagcaccatcggcaccgccaacgccgccgccgccgtgcccaccaccaccgtgctggccgccgccgccgacgaggtgagcgccgccatggccgccctgttcagcggccacgcccaggcctaccaggccctgagcgcccaggccgccctgttccacgagcagttcgtgagggccctgaccgccggcgccggcagctacgccgccgccgaggccgccagcgccgcccccctggagggctga

SEQ ID NO: 10 - protein, 100 aa, synthetic construct
  REGION 1..100: note = Rv1818c (PE_PGRS33)
MSFVVTIPEALAAVATDLAGIGSTIGTANAAAAVPTTTVLAAAADEVSAAMAALFSGHAQAYQALSAQAALFHEQFVRALTAGAGSYAAAEAASAAPLEG

SEQ ID NO: 11 - DNA, 333 bp, synthetic construct
  misc_feature 1..333: note = Rv0159c (PE3)
  source 1..333: mol_type = other DNA
atgagctacgtgatcgccgcccccgagatgctggccaccaccgccgccgacgtggacggcatcggcagcgccatcagggccgccagcgccagcgccgccggccccaccaccggcctgctggccgccgccgccgacgaggtgagcagcgccgccgccgccctgttcagcgagtacgccagggagtgccaggaggtgctgaagcaggccgccgccttccacggcgagttcaccagggccctggccgccgccggcgccgcctacgcccaggccgaggccagcaacaccgccgccatgagcggcaccgccggcagcagcggcgccctgggcagctga

SEQ ID NO: 12 - protein, 110 aa, synthetic construct
  REGION 1..110: note = Rv0159c (PE3)
MSYVIAAPEMLATTAADVDGIGSAIRAASASAAGPTTGLLAAAADEVSSAAAALFSEYARECQEVLKQAAAFHGEFTRALAAAGAAYAQAEASNTAAMSGTAGSSGALGS

SEQ ID NO: 13 - DNA, 333 bp, synthetic construct
  misc_feature 1..333: note = Rv1172c (PE12)
  source 1..333: mol_type = other DNA
atgagcttcgtgttcgccgcccccgaggccctggccgccgccgccgccgacatggccggcatcggcagcaccctgaacgccgccaacgtggtggccgccgtgcccaccaccggcgtgctggccgccgccgccgacgaggtgagcacccaggtggccgccctgctgagcgcccacgcccagggctaccagcagctgagcaggcagatgatgaccgccttccacgaccagttcgtgcaggccctgagggccagcgccgacgcctacgccaccgccgaggccagcgccgcccagaccatggtgaacgccgtgaacgcccccgccagggccctgtga

SEQ ID NO: 14 - protein, 110 aa, synthetic construct
  REGION 1..110: note = Rv1172c (PE12)
MSFVFAAPEALAAAAADMAGIGSTLNAANVVAAVPTTGVLAAAADEVSTQVAALLSAHAQGYQQLSRQMMTAFHDQFVQALRASADAYATAEASAAQTMVNAVNAPARAL

SEQ ID NO: 15 - DNA, 2097 bp, synthetic construct
  misc_feature 1..2097: note = Rv3872-Rv1788-Rv3893c-Rv0285-Rv1818c-Rv0159c-Rv1172 c fusion protein
  source 1..2097: mol_type = other DNA
atggagaagatgagccacgaccccatcgccgccgacatcggcacccaggtgagcgacaacgccctgcacggcgtgaccgccggcagcaccgccctgaccagcgtgaccggcctggtgcccgccggcgccgacgaggtgagcgcccaggccgccaccgccttcaccagcgagggcatccagctgctggccagcaacgccagcgcccaggaccagctgcacagggccggcgaggccgtgcaggacgtggccaggacctacagccagatcgacgacggcgccgccggcgtgttcgccgagatgagcttcgtgaccacccagcccgaggccctggccgccgccgccggcagcctgcagggcatcggcagcgccctgaacgcccagaacgccgccgccgccacccccaccaccggcgtggtgcccgccgccgccgacgaggtgagcgccctgaccgccgcccagttcgccgcccacgcccagatctaccaggccgtgagcgcccaggccgccgccatccacgagatgttcgtgaacaccctgcagatgagcagcggcagctacgccgccaccgaggccgccaacgccgccgccgccggcgccgccgccatggtgtggagcgtgcagcccgaggccgtgctggccagcgccgccgccgagagcgccatcagcgccgagaccgaggccgccgccgccggcgccgcccccgccctgctgagcaccacccccatgggcggcgaccccgacagcgccatgttcagcgccgccctgaacgcctgcggcgccagctacctgggcgtggtggccgagcacgccagccagaggggcctgttcgccggcatgaccctgagggtggtgcccgagggcctggccgccgccagcgccgccgtggaggccctgaccgccaggctggccgccgcccacgccagcgccgcccccgtgatcaccgccgtggtgccccccgccgccgaccccgtgagcctgcagaccgccgccggcttcagcgcccagggcgtggagcacgccgtggtgaccgccgagggcgtggaggagctgggcagggccggcgtgggcgtgggcgagagcggcgccagctacctggccggcgacgccgccgccgccgccacctacggcgtggtgggcggcatgagcttcgtggtgaccatccccgaggccctggccgccgtggccaccgacctggccggcatcggcagcaccatcggcaccgccaacgccgccgccgccgtgcccaccaccaccgtgctggccgccgccgccgacgaggtgagcgccgccatggccgccctgttcagcggccacgcccaggcctaccaggccctgagcgcccaggccgccctgttccacgagcagttcgtgagggccctgaccgccggcgccggcagctacgccgccatgagctacgtgatcgccgcccccgagatgctggccaccaccgccgccgacgtggacggcatcggcagcgccatcagggccgccagcgccagcgccgccggccccaccaccggcctgctggccgccgccgccgacgaggtgagcagcgccgccgccgccctgttcagcgagtacgccagggagtgccaggaggtgctgaagcaggccgccgccttccacggcgagttcaccagggccctggccgccgccggcgccgcctacgcccaggccgaggccagcaacaccgccgccatgagcggcaccgccggcagcagcggcgccctgggcagcatgagcttcgtgttcgccgcccccgaggccctggccgccgccgccgccgacatggccggcatcggcagcaccctgaacgccgccaacgtggtggccgccgtgcccaccaccggcgtgctggccgccgccgccgacgaggtgagcacccaggtggccgccctgctgagcgcccacgcccagggctaccagcagctgagcaggcagatgatgaccgccttccacgaccagttcgtgcaggccctgagggccagcgccgacgcctacgccaccgccgaggccagcgccgcccagaccatggtgaacgccgtgaacgcccccgccagggccctgtacccctacgacgtgcccgactacgcctga

SEQ ID NO: 16 - protein, 698 aa, synthetic construct
  REGION 1..698: note = Rv3872-Rv1788-Rv3893c-Rv0285-Rv1818c-Rv0159c-Rv1172 c fusion protein
MEKMSHDPIAADIGTQVSDNALHGVTAGSTALTSVTGLVPAGADEVSAQAATAFTSEGIQLLASNASAQDQLHRAGEAVQDVARTYSQIDDGAAGVFAEMSFVTTQPEALAAAAGSLQGIGSALNAQNAAAATPTTGVVPAAADEVSALTAAQFAAHAQIYQAVSAQAAAIHEMFVNTLQMSSGSYAATEAANAAAAGAAAMVWSVQPEAVLASAAAESAISAETEAAAAGAAPALLSTTPMGGDPDSAMFSAALNACGASYLGVVAEHASQRGLFAGMTLRVVPEGLAAASAAVEALTARLAAAHASAAPVITAVVPPAADPVSLQTAAGFSAQGVEHAVVTAEGVEELGRAGVGVGESGASYLAGDAAAAATYGVVGGMSFVVTIPEALAAVATDLAGIGSTIGTANAAAAVPTTTVLAAAADEVSAAMAALFSGHAQAYQALSAQAALFHEQFVRALTAGAGSYAAMSYVIAAPEMLATTAADVDGIGSAIRAASASAAGPTTGLLAAAADEVSSAAAALFSEYARECQEVLKQAAAFHGEFTRALAAAGAAYAQAEASNTAAMSGTAGSSGALGSMSFVFAAPEALAAAAADMAGIGSTLNAANVVAAVPTTGVLAAAADEVSTQVAALLSAHAQGYQQLSRQMMTAFHDQFVQALRASADAYATAEASAAQTMVNAVNAPARALYPYDVPDYA

SEQ ID NO: 17 - protein, 9 aa, synthetic construct
  REGION 1..9: note = Hemagglutinin tag
YPYDVPDYA

SEQ ID NO: 18 - DNA, 543 bp, synthetic construct
  misc_feature 1..543: note = Rv3873 (PPE68)
  source 1..543: mol_type = other DNA
atgctgtggcacgccatgccccccgagctgaacaccgccaggctgatggccggcgccggccccgcccccatgctggccgccgccgccggctggcagaccctgagcgccgccctggacgcccaggccgtggagctgaccgccaggctgaacagcctgggcgaggcctggaccggcggcggcagcgacaaggccctggccgccgccacccccatggtggtgtggctgcagaccgccagcacccaggccaagaccagggccatgcaggccaccgcccaggccgccgcctacacccaggccatggccaccacccccagcctgcccgagatcgccgccaaccacatcacccaggccgtgctgaccgccaccaacttcttcggcatcaacaccatccccatcgccctgaccgagatggactacttcatcaggatgtggaaccaggccgccctggccatggaggtgtaccaggccgagaccgccgtgaacaccctgttcgagaagctggagcccatggccagcatcctggaccccggcgccagccagtga

SEQ ID NO: 19 - protein, 180 aa, synthetic construct
  REGION 1..180: note = Rv3873 (PPE68)
MLWHAMPPELNTARLMAGAGPAPMLAAAAGWQTLSAALDAQAVELTARLNSLGEAWTGGGSDKALAAATPMVVWLQTASTQAKTRAMQATAQAAAYTQAMATTPSLPEIAANHITQAVLTATNFFGINTIPIALTEMDYFIRMWNQAALAMEVYQAETAVNTLFEKLEPMASILDPGASQ

SEQ ID NO: 20 - DNA, 543 bp, synthetic construct
  misc_feature 1..543: note = Rv1387 (PPE20)
  source 1..543: mol_type = other DNA
atgaccgagccctggatcgccttcccccccgaggtgcacagcgccatgctgaactacggcgccggcgtgggccccatgctgatcagcgccacccagaacggcgagctgagcgcccagtacgccgaggccgccagcgaggtggaggagctgctgggcgtggtggccagcgagggctggcagggccaggccgccgaggccttcgtggccgcctacatgcccttcctggcctggctgatccaggccagcgccgactgcgtggagatggccgcccagcagcacgtggtgatcgaggcctacaccgccgccgtggagctgatgcccacccaggtggagctggccgccaaccagatcaagctggccgtgctggtggccaccaacttcttcggcatcaacaccatccccatcgccatcaacgaggccgagtacgtggagatgtgggtgagggccgccaccaccatggccacctacagcaccgtgagcaggagcgccctgagcgccatgccccacaccagccccccccccctgatcctgaagagcgactga

SEQ ID NO: 21 - protein, 180 aa, synthetic construct
  REGION 1..180: note = Rv1387 (PPE20)
MTEPWIAFPPEVHSAMLNYGAGVGPMLISATQNGELSAQYAEAASEVEELLGVVASEGWQGQAAEAFVAAYMPFLAWLIQASADCVEMAAQQHVVIEAYTAAVELMPTQVELAANQIKLAVLVATNFFGINTIPIAINEAEYVEMWVRAATTMATYSTVSRSALSAMPHTSPPPLILKSD

SEQ ID NO: 22 - DNA, 543 bp, synthetic construct
  misc_feature 1..543: note = Rv3892c (PPE69)
  source 1..543: mol_type = other DNA
atgcccgaccccggctgggccgccaggacccccgaggccaacgacctgctgctgaccgccggcaccggcgtgggcacccacctggccaaccagaccgcctggaccaccctgggcgccagccaccacgccagcggcgtggccagcgccatcaacaccgccgccaccgccgccagctggctgggcgtgggcagcgccgccagcgccctgaacgtgaccatgctgaacgccaccctgcacggcctggccggctgggtggacgtgaagcccgccgtggtgagcaccgccatcgccgccttcgagaccgccaacgccgccatgaggcccgcccccgagtgcatggagaacagggacgagtggggcgtggacaacgccatcaaccccagcgtgctgtggaccctgacccccaggatcgtgagcctggacgtggagtacttcggcgtgatgtggcccaacaacgccgccgtgggcgccacctacggcggcgtgctggccgccctggccgagagcctggccatccccccccccgtggccaccatgggctga

SEQ ID NO: 23 - protein, 180 aa, synthetic construct
  REGION 1..180: note = Rv3892c (PPE69)
MPDPGWAARTPEANDLLLTAGTGVGTHLANQTAWTTLGASHHASGVASAINTAATAASWLGVGSAASALNVTMLNATLHGLAGWVDVKPAVVSTAIAAFETANAAMRPAPECMENRDEWGVDNAINPSVLWTLTPRIVSLDVEYFGVMWPNNAAVGATYGGVLAALAESLAIPPPVATMG

SEQ ID NO: 24 - DNA, 543 bp, synthetic construct
  misc_feature 1..543: note = Rv1789 (PPE26)
  source 1..543: mol_type = other DNA
atggacttcggcgccctgccccccgaggtgaacagcgtgaggatgtacgccggccccggcagcgcccccatggtggccgccgccagcgcctggaacggcctggccgccgagctgagcagcgccgccaccggctacgagaccgtgatcacccagctgagcagcgagggctggctgggccccgccagcgccgccatggccgaggccgtggccccctacgtggcctggatgagcgccgccgccgcccaggccgagcaggccgccacccaggccagggccgccgccgccgccttcgaggccgccttcgccgccaccgtgcccccccccctgatcgccgccaacagggccagcctgatgcagctgatcagcaccaacgtgttcggccagaacaccagcgccatcgccgccgccgaggcccagtacggcgagatgtgggcccaggacagcgccgccatgtacgcctacgccggcagcagcgccagcgccagcgccgtgacccccttcagcacccccccccagatcgccaaccccaccgcccagggctga

SEQ ID NO: 25 - protein, 180 aa, synthetic construct
  REGION 1..180: note = Rv1789 (PPE26)
MDFGALPPEVNSVRMYAGPGSAPMVAAASAWNGLAAELSSAATGYETVITQLSSEGWLGPASAAMAEAVAPYVAWMSAAAAQAEQAATQARAAAAAFEAAFAATVPPPLIAANRASLMQLISTNVFGQNTSAIAAAEAQYGEMWAQDSAAMYAYAGSSASASAVTPFSTPPQIANPTAQG

SEQ ID NO: 26 - DNA, 543 bp, synthetic construct
  misc_feature 1..543: note = Rv1800 (PPE28)
  source 1..543: mol_type = other DNA
atgctgcccaacttcgccgtgctgccccccgaggtgaacagcgccagggtgttcgccggcgccggcagcgcccccatgctggccgccgccgccgcctgggacgacctggccagcgagctgcactgcgccgccatgagcttcggcagcgtgaccagcggcctggtggtgggctggtggcagggcagcgccagcgccgccatggtggacgccgccgccagctacatcggctggctgagcaccagcgccgcccacgccgagggcgccgccggcctggccagggccgccgtgagcgtgttcgaggaggccctggccgccaccgtgcaccccgccatggtggccgccaacagggcccaggtggccagcctggtggccagcaacctgttcggccagaacgcccccgccatcgccgccctggagagcctgtacgagtgcatgtgggcccaggacgccgccgccatggccggctactacgtgggcgccagcgccgtggccacccagctggccagctggctgcagaggctgcagagcatccccggcgcctga

SEQ ID NO: 27 - protein, 180 aa, synthetic construct
  REGION 1..180: note = Rv1800 (PPE28)
MLPNFAVLPPEVNSARVFAGAGSAPMLAAAAAWDDLASELHCAAMSFGSVTSGLVVGWWQGSASAAMVDAAASYIGWLSTSAAHAEGAAGLARAAVSVFEEALAATVHPAMVAANRAQVASLVASNLFGQNAPAIAALESLYECMWAQDAAAMAGYYVGASAVATQLASWLQRLQSIPGA

SEQ ID NO: 28 - DNA, 543 bp, synthetic construct
  misc_feature 1..543: note = Rv1039c (PPE15)
  source 1..543: mol_type = other DNA
atggacttcggcgccctgccccccgagatcaacagcgccaggatgtacgccggcgccggcgccggccccatgatggccgccggcgccgcctggaacggcctggccgccgagctgggcaccaccgccgccagctacgagagcgtgatcaccaggctgaccaccgagagctggatgggccccgccagcatggccatggtggccgccgcccagccctacctggcctggctgacctacaccgccgaggccgccgcccacgccggcagccaggccatggccagcgccgccgcctacgaggccgcctacgccatgaccgtgccccccgaggtggtggccgccaacagggccctgctggccgccctggtggccaccaacgtgctgggcatcaacacccccgccatcatggccaccgaggccctgtacgccgagatgtgggcccaggacgccctggccatgtacggctacgccgccgccagcggcgccgccggcatgctgcagcccctgagcccccccagccagaccaccaaccccggcggcctggcctga

SEQ ID NO: 29 - protein, 180 aa, synthetic construct
  REGION 1..180: note = Rv1039c (PPE15)
MDFGALPPEINSARMYAGAGAGPMMAAGAAWNGLAAELGTTAASYESVITRLTTESWMGPASMAMVAAAQPYLAWLTYTAEAAAHAGSQAMASAAAYEAAYAMTVPPEVVAANRALLAALVATNVLGINTPAIMATEALYAEMWAQDALAMYGYAAASGAAGMLQPLSPPSQTTNPGGLA

SEQ ID NO: 30 - DNA, 1650 bp, synthetic construct
  misc_feature 1..1650: note = Rv3873-Rv1387-Rv3892c fusion protein
  source 1..1650: mol_type = other DNA
atgctgtggcacgccatgccccccgagctgaacaccgccaggctgatggccggcgccggccccgcccccatgctggccgccgccgccggctggcagaccctgagcgccgccctggacgcccaggccgtggagctgaccgccaggctgaacagcctgggcgaggcctggaccggcggcggcagcgacaaggccctggccgccgccacccccatggtggtgtggctgcagaccgccagcacccaggccaagaccagggccatgcaggccaccgcccaggccgccgcctacacccaggccatggccaccacccccagcctgcccgagatcgccgccaaccacatcacccaggccgtgctgaccgccaccaacttcttcggcatcaacaccatccccatcgccctgaccgagatggactacttcatcaggatgtggaaccaggccgccctggccatggaggtgtaccaggccgagaccgccgtgaacaccctgttcgagaagctggagcccatggccagcatcctggaccccggcgccagccagatgaccgagccctggatcgccttcccccccgaggtgcacagcgccatgctgaactacggcgccggcgtgggccccatgctgatcagcgccacccagaacggcgagctgagcgcccagtacgccgaggccgccagcgaggtggaggagctgctgggcgtggtggccagcgagggctggcagggccaggccgccgaggccttcgtggccgcctacatgcccttcctggcctggctgatccaggccagcgccgactgcgtggagatggccgcccagcagcacgtggtgatcgaggcctacaccgccgccgtggagctgatgcccacccaggtggagctggccgccaaccagatcaagctggccgtgctggtggccaccaacttcttcggcatcaacaccatccccatcgccatcaacgaggccgagtacgtggagatgtgggtgagggccgccaccaccatggccacctacagcaccgtgagcaggagcgccctgagcgccatgccccacaccagccccccccccctgatcctgaagagcgacatgcccgaccccggctgggccgccaggacccccgaggccaacgacctgctgctgaccgccggcaccggcgtgggcacccacctggccaaccagaccgcctggaccaccctgggcgccagccaccacgccagcggcgtggccagcgccatcaacaccgccgccaccgccgccagctggctgggcgtgggcagcgccgccagcgccctgaacgtgaccatgctgaacgccaccctgcacggcctggccggctgggtggacgtgaagcccgccgtggtgagcaccgccatcgccgccttcgagaccgccaacgccgccatgaggcccgcccccgagtgcatggagaacagggacgagtggggcgtggacaacgccatcaaccccagcgtgctgtggaccctgacccccaggatcgtgagcctggacgtggagtacttcggcgtgatgtggcccaacaacgccgccgtgggcgccacctacggcggcgtgctggccgccctggccgagagcctggccatccccccccccgtggccaccatgggctacccctacgacgtgcccgactacgcctga

SEQ ID NO: 31 - protein, 549 aa, synthetic construct
  REGION 1..549: note = Rv3873-Rv1387-Rv3892c fusion protein
MLWHAMPPELNTARLMAGAGPAPMLAAAAGWQTLSAALDAQAVELTARLNSLGEAWTGGGSDKALAAATPMVVWLQTASTQAKTRAMQATAQAAAYTQAMATTPSLPEIAANHITQAVLTATNFFGINTIPIALTEMDYFIRMWNQAALAMEVYQAETAVNTLFEKLEPMASILDPGASQMTEPWIAFPPEVHSAMLNYGAGVGPMLISATQNGELSAQYAEAASEVEELLGVVASEGWQGQAAEAFVAAYMPFLAWLIQASADCVEMAAQQHVVIEAYTAAVELMPTQVELAANQIKLAVLVATNFFGINTIPIAINEAEYVEMWVRAATTMATYSTVSRSALSAMPHTSPPPLILKSDMPDPGWAARTPEANDLLLTAGTGVGTHLANQTAWTTLGASHHASGVASAINTAATAASWLGVGSAASALNVTMLNATLHGLAGWVDVKPAVVSTAIAAFETANAAMRPAPECMENRDEWGVDNAINPSVLWTLTPRIVSLDVEYFGVMWPNNAAVGATYGGVLAALAESLAIPPPVATMGYPYDVPDYA

SEQ ID NO: 32 - DNA, 1650 bp, synthetic construct
  misc_feature 1..1650: note = Rv1789-Rv1800-Rv1039c fusion protein
  source 1..1650: mol_type = other DNA
atggacttcggcgccctgccccccgaggtgaacagcgtgaggatgtacgccggccccggcagcgcccccatggtggccgccgccagcgcctggaacggcctggccgccgagctgagcagcgccgccaccggctacgagaccgtgatcacccagctgagcagcgagggctggctgggccccgccagcgccgccatggccgaggccgtggccccctacgtggcctggatgagcgccgccgccgcccaggccgagcaggccgccacccaggccagggccgccgccgccgccttcgaggccgccttcgccgccaccgtgcccccccccctgatcgccgccaacagggccagcctgatgcagctgatcagcaccaacgtgttcggccagaacaccagcgccatcgccgccgccgaggcccagtacggcgagatgtgggcccaggacagcgccgccatgtacgcctacgccggcagcagcgccagcgccagcgccgtgacccccttcagcacccccccccagatcgccaaccccaccgcccagggcatgctgcccaacttcgccgtgctgccccccgaggtgaacagcgccagggtgttcgccggcgccggcagcgcccccatgctggccgccgccgccgcctgggacgacctggccagcgagctgcactgcgccgccatgagcttcggcagcgtgaccagcggcctggtggtgggctggtggcagggcagcgccagcgccgccatggtggacgccgccgccagctacatcggctggctgagcaccagcgccgcccacgccgagggcgccgccggcctggccagggccgccgtgagcgtgttcgaggaggccctggccgccaccgtgcaccccgccatggtggccgccaacagggcccaggtggccagcctggtggccagcaacctgttcggccagaacgcccccgccatcgccgccctggagagcctgtacgagtgcatgtgggcccaggacgccgccgccatggccggctactacgtgggcgccagcgccgtggccacccagctggccagctggctgcagaggctgcagagcatccccggcgccatggacttcggcgccctgccccccgagatcaacagcgccaggatgtacgccggcgccggcgccggccccatgatggccgccggcgccgcctggaacggcctggccgccgagctgggcaccaccgccgccagctacgagagcgtgatcaccaggctgaccaccgagagctggatgggccccgccagcatggccatggtggccgccgcccagccctacctggcctggctgacctacaccgccgaggccgccgcccacgccggcagccaggccatggccagcgccgccgcctacgaggccgcctacgccatgaccgtgccccccgaggtggtggccgccaacagggccctgctggccgccctggtggccaccaacgtgctgggcatcaacacccccgccatcatggccaccgaggccctgtacgccgagatgtgggcccaggacgccctggccatgtacggctacgccgccgccagcggcgccgccggcatgctgcagcccctgagcccccccagccagaccaccaaccccggcggcctggcctacccctacgacgtgcccgactacgcctga

SEQ ID NO: 33 - protein, 549 aa, synthetic construct
  REGION 1..549: note = Rv1789-Rv1800-Rv1039c fusion protein
MDFGALPPEVNSVRMYAGPGSAPMVAAASAWNGLAAELSSAATGYETVITQLSSEGWLGPASAAMAEAVAPYVAWMSAAAAQAEQAATQARAAAAAFEAAFAATVPPPLIAANRASLMQLISTNVFGQNTSAIAAAEAQYGEMWAQDSAAMYAYAGSSASASAVTPFSTPPQIANPTAQGMLPNFAVLPPEVNSARVFAGAGSAPMLAAAAAWDDLASELHCAAMSFGSVTSGLVVGWWQGSASAAMVDAAASYIGWLSTSAAHAEGAAGLARAAVSVFEEALAATVHPAMVAANRAQVASLVASNLFGQNAPAIAALESLYECMWAQDAAAMAGYYVGASAVATQLASWLQRLQSIPGAMDFGALPPEINSARMYAGAGAGPMMAAGAAWNGLAAELGTTAASYESVITRLTTESWMGPASMAMVAAAQPYLAWLTYTAEAAAHAGSQAMASAAAYEAAYAMTVPPEVVAANRALLAALVATNVLGINTPAIMATEALYAEMWAQDALAMYGYAAASGAAGMLQPLSPPSQTTNPGGLAYPYDVPDYA

SEQ ID NO: 34 - DNA, 363 bp, synthetic construct
  misc_feature 1..363: note = Rv3017c (esxQ)
  source 1..363: mol_type = other DNA
gtgagccagagcatgtacagctaccccgccatgaccgccaacgtgggcgacatggccggctacaccggcaccacccagagcctgggcgccgacatcgccagcgagaggaccgcccccagcagggcctgccagggcgacctgggcatgagccaccaggactggcaggcccagtggaaccaggccatggaggccctggccagggcctacaggaggtgcaggagggccctgaggcagatcggcgtgctggagaggcccgtgggcgacagcagcgactgcggcaccatcagggtgggcagcttcaggggcaggtggctggaccccaggcacgccggccccgccaccgccgccgacgccggcgactga

SEQ ID NO: 35 - protein, 120 aa, synthetic construct
  REGION 1..120: note = Rv3017c (esxQ)
VSQSMYSYPAMTANVGDMAGYTGTTQSLGADIASERTAPSRACQGDLGMSHQDWQAQWNQAMEALARAYRRCRRALRQIGVLERPVGDSSDCGTIRVGSFRGRWLDPRHAGPATAADAGD

SEQ ID NO: 36 - DNA, 294 bp, synthetic construct
  misc_feature 1..294: note = Rv3020c (esxS)
  source 1..294: mol_type = other DNA
atgagcctgctggacgcccacatcccccagctgatcgccagccacaccgccttcgccgccaaggccggcctgatgaggcacaccatcggccaggccgagcagcaggccatgagcgcccaggccttccaccagggcgagagcgccgccgccttccagggcgcccacgccaggttcgtggccgccgccgccaaggtgaacaccctgctggacatcgcccaggccaacctgggcgaggccgccggcacctacgtggccgccgacgccgccgccgccagcagctacaccggcttctga

SEQ ID NO: 37 - protein, 97 aa, synthetic construct
  REGION 1..97: note = Rv3020c (esxS)
MSLLDAHIPQLIASHTAFAAKAGLMRHTIGQAEQQAMSAQAFHQGESAAAFQGAHARFVAAAAKVNTLLDIAQANLGEAAGTYVAADAAAASSYTGF

SEQ ID NO: 38 - DNA, 291 bp, synthetic construct
  misc_feature 1..291: note = Rv3019c (esxR)
  source 1..291: mol_type = other DNA
atgagccagatcatgtacaactaccccgccatgatggcccacgccggcgacatggccggctacgccggcaccctgcagagcctgggcgccgacatcgccagcgagcaggccgtgctgagcagcgcctggcagggcgacaccggcatcacctaccagggctggcagacccagtggaaccaggccctggaggacctggtgagggcctaccagagcatgagcggcacccacgagagcaacaccatggccatgctggccagggacggcgccgaggccgccaagtggggcggctga

SEQ ID NO: 39 - protein, 96 aa, synthetic construct
  REGION 1..96: note = Rv3019c (esxR)
MSQIMYNYPAMMAHAGDMAGYAGTLQSLGADIASEQAVLSSAWQGDTGITYQGWQTQWNQALEDLVRAYQSMSGTHESNTMAMLARDGAEAAKWGG

SEQ ID NO: 40 - DNA, 324 bp, synthetic construct
  misc_feature 1..324: note = Rv3891c (esxD)
  source 1..324: mol_type = other DNA
gtggccgacaccatccaggtgaccccccagatgctgaggagcaccgccaacgacatccaggccaacatggagcaggccatgggcatcgccaagggctacctggccaaccaggagaacgtgatgaaccccgccacctggagcggcaccggcgtggtggccagccacatgaccgccaccgagatcaccaacgagctgaacaaggtgctgaccggcggcaccaggctggccgagggcctggtgcaggccgccgccctgatggagggccacgaggccgacagccagaccgccttccaggccctgttcggcgccagccacggcagctga

SEQ ID NO: 41 - protein, 107 aa, synthetic construct
  REGION 1..107: note = Rv3891c (esxD)
VADTIQVTPQMLRSTANDIQANMEQAMGIAKGYLANQENVMNPATWSGTGVVASHMTATEITNELNKVLTGGTRLAEGLVQAAALMEGHEADSQTAFQALFGASHGS

SEQ ID NO: 42 - DNA, 285 bp, synthetic construct
  misc_feature 1..285: note = Rv2346c (esxO)
  source 1..285: mol_type = other DNA
atgaccatcaactaccagttcggcgacgtggacgcccacggcgccatgatcagggcccaggccggcctgctggaggccgagcaccaggccatcgtgagggacgtgctggccgccggcgacttctggggcggcgccggcagcgtggcctgccaggagttcatcacccagctgggcaggaacttccaggtgatctacgagcaggccaacgcccacggccagaaggtgcaggccgccggcaacaacatggcccagaccgacagcgccgtgggcagcagctgggcctga

SEQ ID NO: 43 - protein, 94 aa, synthetic construct
  REGION 1..94: note = Rv2346c (esxO)
MTINYQFGDVDAHGAMIRAQAGLLEAEHQAIVRDVLAAGDFWGGAGSVACQEFITQLGRNFQVIYEQANAHGQKVQAAGNNMAQTDSAVGSSWA

SEQ ID NO: 44 - DNA, 318 bp, synthetic construct
  misc_feature 1..318: note = Rv3445c (esxU)
  source 1..318: mol_type = other DNA
gtgagcacccccaacaccctgaacgccgacttcgacctgatgaggagcgtggccggcatcaccgacgccaggaacgaggagatcagggccatgctgcaggccttcatcggcaggatgagcggcgtgccccccagcgtgtggggcggcctggccgccgccaggttccaggacgtggtggacaggtggaacgccgagagcaccaggctgtaccacgtgctgcacgccatcgccgacaccatcaggcacaacgaggccgccctgagggaggccggccagatccacgccaggcacatcgccgccgccggcggcgacctgtga

SEQ ID NO: 45 - protein, 105 aa, synthetic construct
  REGION 1..105: note = Rv3445c (esxU)
VSTPNTLNADFDLMRSVAGITDARNEEIRAMLQAFIGRMSGVPPSVWGGLAAARFQDVVDRWNAESTRLYHVLHAIADTIRHNEAALREAGQIHARHIAAAGGDL

SEQ ID NO: 46 - DNA, 285 bp, synthetic construct
  misc_feature 1..285: note = Rv3619c (esxV)
  source 1..285: mol_type = other DNA
atgaccatcaactaccagttcggcgacgtggacgcccacggcgccatgatcagggcccaggccggcagcctggaggccgagcaccaggccatcatcagcgacgtgctgaccgccagcgacttctggggcggcgccggcagcgccgcctgccagggcttcatcacccagctgggcaggaacttccaggtgatctacgagcaggccaacgcccacggccagaaggtgcaggccgccggcaacaacatggcccagaccgacagcgccgtgggcagcagctgggcctga

SEQ ID NO: 47 - protein, 94 aa, synthetic construct
  REGION 1..94: note = Rv3619c (esxV)
MTINYQFGDVDAHGAMIRAQAGSLEAEHQAIISDVLTASDFWGGAGSAACQGFITQLGRNFQVIYEQANAHGQKVQAAGNNMAQTDSAVGSSWA

SEQ ID NO: 48 - DNA, 288 bp, synthetic construct
  misc_feature 1..288: note = Rv3875 (esxA, ESAT6)
  source 1..288: mol_type = other DNA
atgaccgagcagcagtggaacttcgccggcatcgaggccgccgccagcgccatccagggcaacgtgaccagcatccacagcctgctggacgagggcaagcagagcctgaccaagctggccgccgcctggggcggcagcggcagcgaggcctaccagggcgtgcagcagaagtgggacgccaccgccaccgagctgaacaacgccctgcagaacctggccaggaccatcagcgaggccggccaggccatggccagcaccgagggcaacgtgaccggcatgttcgcctga

SEQ ID NO: 49 - protein, 95 aa, synthetic construct
  REGION 1..95: note = Rv3875 (esxA, ESAT6)
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 50 - DNA, 303 bp, synthetic construct
  misc_feature 1..303: note = Rv3874 (esxB, CFP10)
  source 1..303: mol_type = other DNA
atggccgagatgaagaccgacgccgccaccctggcccaggaggccggcaacttcgagaggatcagcggcgacctgaagacccagatcgaccaggtggagagcaccgccggcagcctgcagggccagtggaggggcgccgccggcaccgccgcccaggccgccgtggtgaggttccaggaggccgccaacaagcagaagcaggagctggacgagatcagcaccaacatcaggcaggccggcgtgcagtacagcagggccgacgaggagcagcagcaggccctgagcagccagatgggcttctga

SEQ ID NO: 51 - protein, 100 aa, synthetic construct
  REGION 1..100: note = Rv3874 (esxB, CFP10)
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 52 - DNA, 1887 bp, synthetic construct
  misc_feature 1..1887: note = Rv3017c-Rv3020c-Rv3019c-Rv3891c-Rv2346c-Rv3445c fusion protein
  source 1..1887: mol_type = other DNA
gtgagccagagcatgtacagctaccccgccatgaccgccaacgtgggcgacatggccggctacaccggcaccacccagagcctgggcgccgacatcgccagcgagaggaccgcccccagcagggcctgccagggcgacctgggcatgagccaccaggactggcaggcccagtggaaccaggccatggaggccctggccagggcctacaggaggtgcaggagggccctgaggcagatcggcgtgctggagaggcccgtgggcgacagcagcgactgcggcaccatcagggtgggcagcttcaggggcaggtggctggaccccaggcacgccggccccgccaccgccgccgacgccggcgacatgagcctgctggacgcccacatcccccagctgatcgccagccacaccgccttcgccgccaaggccggcctgatgaggcacaccatcggccaggccgagcagcaggccatgagcgcccaggccttccaccagggcgagagcgccgccgccttccagggcgcccacgccaggttcgtggccgccgccgccaaggtgaacaccctgctggacatcgcccaggccaacctgggcgaggccgccggcacctacgtggccgccgacgccgccgccgccagcagctacaccggcttcatgagccagatcatgtacaactaccccgccatgatggcccacgccggcgacatggccggctacgccggcaccctgcagagcctgggcgccgacatcgccagcgagcaggccgtgctgagcagcgcctggcagggcgacaccggcatcacctaccagggctggcagacccagtggaaccaggccctggaggacctggtgagggcctaccagagcatgagcggcacccacgagagcaacaccatggccatgctggccagggacggcgccgaggccgccaagtggggcggcgtggccgacaccatccaggtgaccccccagatgctgaggagcaccgccaacgacatccaggccaacatggagcaggccatgggcatcgccaagggctacctggccaaccaggagaacgtgatgaaccccgccacctggagcggcaccggcgtggtggccagccacatgaccgccaccgagatcaccaacgagctgaacaaggtgctgaccggcggcaccaggctggccgagggcctggtgcaggccgccgccctgatggagggccacgaggccgacagccagaccgccttccaggccctgttcggcgccagccacggcagcatgaccatcaactaccagttcggcgacgtggacgcccacggcgccatgatcagggcccaggccggcctgctggaggccgagcaccaggccatcgtgagggacgtgctggccgccggcgacttctggggcggcgccggcagcgtggcctgccaggagttcatcacccagctgggcaggaacttccaggtgatctacgagcaggccaacgcccacggccagaaggtgcaggccgccggcaacaacatggcccagaccgacagcgccgtgggcagcagctgggccgtgagcacccccaacaccctgaacgccgacttcgacctgatgaggagcgtggccggcatcaccgacgccaggaacgaggagatcagggccatgctgcaggccttcatcggcaggatgagcggcgtgccccccagcgtgtggggcggcctggccgccgccaggttccaggacgtggtggacaggtggaacgccgagagcaccaggctgtaccacgtgctgcacgccatcgccgacaccatcaggcacaacgaggccgccctgagggaggccggccagatccacgccaggcacatcgccgccgccggcggcgacctgtacccctacgacgtgcccgactacgcctga

SEQ ID NO: 53 - protein, 628 aa, synthetic construct
  REGION 1..628: note = Rv3017c-Rv3020c-Rv3019c-Rv3891c-Rv2346c-Rv3445c fusion protein
VSQSMYSYPAMTANVGDMAGYTGTTQSLGADIASERTAPSRACQGDLGMSHQDWQAQWNQAMEALARAYRRCRRALRQIGVLERPVGDSSDCGTIRVGSFRGRWLDPRHAGPATAADAGDMSLLDAHIPQLIASHTAFAAKAGLMRHTIGQAEQQAMSAQAFHQGESAAAFQGAHARFVAAAAKVNTLLDIAQANLGEAAGTYVAADAAAASSYTGFMSQIMYNYPAMMAHAGDMAGYAGTLQSLGADIASEQAVLSSAWQGDTGITYQGWQTQWNQALEDLVRAYQSMSGTHESNTMAMLARDGAEAAKWGGVADTIQVTPQMLRSTANDIQANMEQAMGIAKGYLANQENVMNPATWSGTGVVASHMTATEITNELNKVLTGGTRLAEGLVQAAALMEGHEADSQTAFQALFGASHGSMTINYQFGDVDAHGAMIRAQAGLLEAEHQAIVRDVLAAGDFWGGAGSVACQEFITQLGRNFQVIYEQANAHGQKVQAAGNNMAQTDSAVGSSWAVSTPNTLNADFDLMRSVAGITDARNEEIRAMLQAFIGRMSGVPPSVWGGLAAARFQDVVDRWNAESTRLYHVLHAIADTIRHNEAALREAGQIHARHIAAAGGDLYPYDVPDYA

SEQ ID NO: 54 - DNA, 897 bp, synthetic construct
  misc_feature 1..897: note = Rv3619c-Rv3875-Rv3874 fusion protein
  source 1..897: mol_type = other DNA
atgaccatcaactaccagttcggcgacgtggacgcccacggcgccatgatcagggcccaggccggcagcctggaggccgagcaccaggccatcatcagcgacgtgctgaccgccagcgacttctggggcggcgccggcagcgccgcctgccagggcttcatcacccagctgggcaggaacttccaggtgatctacgagcaggccaacgcccacggccagaaggtgcaggccgccggcaacaacatggcccagaccgacagcgccgtgggcagcagctgggccatgaccgagcagcagtggaacttcgccggcatcgaggccgccgccagcgccatccagggcaacgtgaccagcatccacagcctgctggacgagggcaagcagagcctgaccaagctggccgccgcctggggcggcagcggcagcgaggcctaccagggcgtgcagcagaagtgggacgccaccgccaccgagctgaacaacgccctgcagaacctggccaggaccatcagcgaggccggccaggccatggccagcaccgagggcaacgtgaccggcatgttcgccatggccgagatgaagaccgacgccgccaccctggcccaggaggccggcaacttcgagaggatcagcggcgacctgaagacccagatcgaccaggtggagagcaccgccggcagcctgcagggccagtggaggggcgccgccggcaccgccgcccaggccgccgtggtgaggttccaggaggccgccaacaagcagaagcaggagctggacgagatcagcaccaacatcaggcaggccggcgtgcagtacagcagggccgacgaggagcagcagcaggccctgagcagccagatgggcttctacccctacgacgtgcccgactacgcctga

SEQ ID NO: 55 - protein, 298 aa, synthetic construct
  REGION 1..298: note = Rv3619c-Rv3875-Rv3874 fusion protein
MTINYQFGDVDAHGAMIRAQAGSLEAEHQAIISDVLTASDFWGGAGSAACQGFITQLGRNFQVIYEQANAHGQKVQAAGNNMAQTDSAVGSSWAMTEQQWNFAGIEAAASAIQGNVTSIHSLLDEGKQSLTKLAAAWGGSGSEAYQGVQQKWDATATELNNALQNLARTISEAGQAMASTEGNVTGMFAMAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFYPYDVPDYA

SEQ ID NO: 56 - DNA, 498 bp, synthetic construct
  misc_feature 1..498: note = Rv2719c
  source 1..498: mol_type = other DNA
atgacccccgtgaggcccccccacacccccgaccccctgaacctgaggggccccctggacggccccaggtggaggagggccgagcccgcccagagcaggaggcccggcaggagcaggcccggcggcgcccccctgaggtaccacaggaccggcgtgggcatgagcaggaccggccacggcagcaggcccgtgccccccgccaccaccgtgggcctggccctgctggccgccgccatcaccctgtggctgggcctggtggcccagttcggccagatgatcaccggcggcagcgccgacggcagcgccgacagcaccggcagggtgcccgacaggctggccgtggtgagggtggagaccggcgagagcctgtacgacgtggccgtgagggtggcccccaacgcccccaccaggcaggtggccgacaggatcagggagctgaacggcctgcagacccccgccctggccgtgggccagaccctgatcgcccccgtgggctga

SEQ ID NO: 57 - protein, 165 aa, synthetic construct
  REGION 1..165: note = Rv2719c
MTPVRPPHTPDPLNLRGPLDGPRWRRAEPAQSRRPGRSRPGGAPLRYHRTGVGMSRTGHGSRPVPPATTVGLALLAAAITLWLGLVAQFGQMITGGSADGSADSTGRVPDRLAVVRVETGESLYDVAVRVAPNAPTRQVADRIRELNGLQTPALAVGQTLIAPVG

SEQ ID NO: 58 - DNA, 426 bp, synthetic construct
  misc_feature 1..426: note = Rv0010c
  source 1..426: mol_type = other DNA
atgcagcagaccgcctgggcccccaggaccagcggcatcgccggctgcggcgccggcggcgtggtgatggccatcgccagcgtgaccctggtgaccgacacccccggcagggtgctgaccggcgtggccgccctgggcctgatcctgttcgccagcgccacctggagggccaggcccaggctggccatcacccccgacggcctggccatcaggggctggttcaggacccagctgctgaggcacagcaacatcaagatcatcaggatcgacgagttcaggaggtacggcaggctggtgaggctgctggagatcgagaccgtgagcggcggcctgctgatcctgagcaggtgggacctgggcaccgaccccgtggaggtgctggacgccctgaccgccgccggctacgccggcaggggccagaggtga

SEQ ID NO: 59 - protein, 141 aa, synthetic construct
  REGION 1..141: note = Rv0010c
MQQTAWAPRTSGIAGCGAGGVVMAIASVTLVTDTPGRVLTGVAALGLILFASATWRARPRLAITPDGLAIRGWFRTQLLRHSNIKIIRIDEFRRYGRLVRLLEIETVSGGLLILSRWDLGTDPVEVLDALTAAGYAGRGQR

SEQ ID NO: 60 - DNA, 1245 bp, synthetic construct
  misc_feature 1..1245: note = Rv1872c
  source 1..1245: mol_type = other DNA
atggccgtgaacaggagggtgcccagggtgagggacctggcccccctgctgcagttcaacaggccccagttcgacaccagcaagaggaggctgggcgccgccctgaccatccaggacctgaggaggatcgccaagaggaggacccccagggccgccttcgactacgccgacggcggcgccgaggacgagctgagcatcgccagggccaggcagggcttcagggacatcgagttccaccccaccatcctgagggacgtgaccaccgtgtgcgccggctggaacgtgctgggccagcccaccgtgctgcccttcggcatcgcccccaccggcttcaccaggctgatgcacaccgagggcgagatcgccggcgccagggccgccgccgccgccggcatccccttcagcctgagcaccctggccacctgcgccatcgaggacctggtgatcgccgtgccccagggcaggaagtggttccagctgtacatgtggagggacagggacaggagcatggccctggtgaggagggtggccgccgccggcttcgacaccatgctggtgaccgtggacgtgcccgtggccggcgccaggctgagggacgtgaggaacggcatgagcatcccccccgccctgaccctgaggaccgtgctggacgccatgggccaccccaggtggtggttcgacctgctgaccaccgagcccctggccttcgccagcctggacaggtggcccggcaccgtgggcgagtacctgaacaccgtgttcgaccccagcctgaccttcgacgacctggcctggatcaagagccagtggcccggcaagctggtggtgaagggcatccagaccctggacgacgccagggccgtggtggacaggggcgtggacggcatcgtgctgagcaaccacggcggcaggcagctggacagggcccccgtgcccttccacctgctgccccacgtggccagggagctgggcaagcacaccgagatcctggtggacaccggcatcatgagcggcgccgacatcgtggccgccatcgccctgggcgccaggtgcaccctgatcggcagggcctacctgtacggcctgatggccggcggcgaggccggcgtgaacagggccatcgagatcctgcagaccggcgtgatcaggaccatgaggctgctgggcgtgacctgcctggaggagctgagccccaggcacgtgacccagctgaggaggctgggccccatcggcgcccccacctga

SEQ ID NO: 61 - protein, 414 aa, synthetic construct
  REGION 1..414: note = Rv1872c
MAVNRRVPRVRDLAPLLQFNRPQFDTSKRRLGAALTIQDLRRIAKRRTPRAAFDYADGGAEDELSIARARQGFRDIEFHPTILRDVTTVCAGWNVLGQPTVLPFGIAPTGFTRLMHTEGEIAGARAAAAAGIPFSLSTLATCAIEDLVIAVPQGRKWFQLYMWRDRDRSMALVRRVAAAGFDTMLVTVDVPVAGARLRDVRNGMSIPPALTLRTVLDAMGHPRWWFDLLTTEPLAFASLDRWPGTVGEYLNTVFDPSLTFDDLAWIKSQWPGKLVVKGIQTLDDARAVVDRGVDGIVLSNHGGRQLDRAPVPFHLLPHVARELGKHTEILVDTGIMSGADIVAAIALGARCTLIGRAYLYGLMAGGEAGVNRAIEILQTGVIRTMRLLGVTCLEELSPRHVTQLRRLGPIGAPT

SEQ ID NO: 62 - DNA, 789 bp, synthetic construct
  misc_feature 1..789: note = Rv0012
  source 1..789: mol_type = other DNA
atgaggctgacccaccccaccccctgccccgagaacggcgagaccatgatcgacaggaggaggagcgcctggaggttcagcgtgcccctggtgtgcctgctggccggcctgctgctggccgccacccacggcgtgagcggcggcaccgagatcaggaggagcgacgcccccaggctggtggacctggtgaggagggcccaggccagcgtgaacaggctggccaccgagagggaggccctgaccaccaggatcgacagcgtgcacggcaggagcgtggacaccgccctggccgccatgcagaggaggagcgccaagctggccggcgtggccgccatgaaccccgtgcacggccccggcctggtggtgaccctgcaggacgcccagagggacgccaacggcaggttccccagggacgccagccccgacgacctggtggtgcaccagcaggacatcgaggccgtgctgaacgccctgtggaacgccggcgccgaggccatccagatgcaggaccagaggatcatcgccatgagcatcgccaggtgcgtgggcaacaccctgctgctgaacggcaggacctacagccccccctacaccatcgccgccatcggcgacgccgccgccatgcaggccgccctggccgccgcccccctggtgaccctgtacaagcagtacgtggtgaggttcggcctgggctactgcgaggaggtgcaccccgacctgcagatcgtgggctacgccgaccccgtgaggatgcacttcgcccagcccgccggccccctggactactga

SEQ ID NO: 63 - protein, 262 aa, synthetic construct
  REGION 1..262: note = Rv0012
MRLTHPTPCPENGETMIDRRRSAWRFSVPLVCLLAGLLLAATHGVSGGTEIRRSDAPRLVDLVRRAQASVNRLATEREALTTRIDSVHGRSVDTALAAMQRRSAKLAGVAAMNPVHGPGLVVTLQDAQRDANGRFPRDASPDDLVVHQQDIEAVLNALWNAGAEAIQMQDQRIIAMSIARCVGNTLLLNGRTYSPPYTIAAIGDAAAMQAALAAAPLVTLYKQYVVRFGLGYCEEVHPDLQIVGYADPVRMHFAQPAGPLDY

SEQ ID NO: 64 - DNA, 657 bp, synthetic construct
  misc_feature 1..657: note = Rv0990c
  source 1..657: mol_type = other DNA
gtggccgagagcagcctgaaccccagcctggtgagcaggatcagcgccttcctgaggcccgactggaccaggaccgtgagggccaggaggttcgccgccgccggcctggtgatgctggccggcgtggccgccctgaggagcaaccccgaggacgacaggagcgaggtggtggtggccgcccacgacctgaggcccggcaccgccctgacccccggcgacgtgaggctggagaagaggagcgccaccaccctgcccgacggcagccaggccgacctggacgccgtggtgggcagcaccctggccagccccaccaggaggggcgaggtgctgaccgacgtgaggctgctgggcagcaggctggccgagagcaccgccggccccgacgccaggatcgtgcccctgcacctggccgacagcgccctggtggacctggtgagggtgggcgacgtggtggacgtgctggccgcccccgtgaccgacagccccgccgccctgaggctgctggccaccgacgccatcgtggtgctggtgagcgcccagcagaaggcccaggccgccgacagcgacagggtggtgctggtggccctgcccgccaggctggccaacaccgtggccggcgccgccctgggccagaccgtgaccctgaccctgcactga

SEQ ID NO: 65 - protein, 218 aa, synthetic construct
  REGION 1..218: note = Rv0990c
VAESSLNPSLVSRISAFLRPDWTRTVRARRFAAAGLVMLAGVAALRSNPEDDRSEVVVAAHDLRPGTALTPGDVRLEKRSATTLPDGSQADLDAVVGSTLASPTRRGEVLTDVRLLGSRLAESTAGPDARIVPLHLADSALVDLVRVGDVVDVLAAPVTDSPAALRLLATDAIVVLVSAQQKAQAADSDRVVLVALPARLANTVAGAALGQTVTLTLH

SEQ ID NO: 66 - DNA, 612 bp, synthetic construct
  misc_feature 1..612: note = Rv0995
  source 1..612: mol_type = other DNA
atggccgtgggccccctgagggtgagcgccggcgtgatcaggctgaggcccgtgaggatgagggacggcgtgcactggagcaggatcaggctggccgacagggcccacctggagccctgggagcccagcgccgacggcgagtggaccgtgaggcacaccgtggccgcctggcccgccgtgtgcagcggcctgaggagcgaggccaggaacggcaggatgctgccctacgtgatcgagctggacggccagttctgcggccagctgaccatcggcaacgtgacccacggcgccctgaggagcgcctggatcggctactgggtgcccagcgccgccaccggcggcggcgtggccaccggcgccctggccctgggcctggaccactgcttcggccccgtgatgctgcacagggtggaggccaccgtgaggcccgagaacgccgccagcagggccgtgctggccaaggtgggcttcagggaggagggcctgctgaggaggtacctggaggtggacagggcctggagggaccacctgctgatggccatcaccgtggaggaggtgtacggcagcgtggccagcaccctggtgagggccggccacgccagctggccctga

SEQ ID NO: 67 - protein, 203 aa, synthetic construct
  REGION 1..203: note = Rv0995
MAVGPLRVSAGVIRLRPVRMRDGVHWSRIRLADRAHLEPWEPSADGEWTVRHTVAAWPAVCSGLRSEARNGRMLPYVIELDGQFCGQLTIGNVTHGALRSAWIGYWVPSAATGGGVATGALALGLDHCFGPVMLHRVEATVRPENAASRAVLAKVGFREEGLLRRYLEVDRAWRDHLLMAITVEEVYGSVASTLVRAGHASWP

SEQ ID NO: 68 - DNA, 2190 bp, synthetic construct
  misc_feature 1..2190: note = Rv2719c-Rv0010c-Rv1872c fusion protein
  source 1..2190: mol_type = other DNA
atgacccccgtgaggcccccccacacccccgaccccctgaacctgaggggccccctggacggccccaggtggaggagggccgagcccgcccagagcaggaggcccggcaggagcaggcccggcggcgcccccctgaggtaccacaggaccggcgtgggcatgagcaggaccggccacggcagcaggcccgtgccccccgccaccaccgtgggcctggccctgctggccgccgccatcaccctgtggctgggcctggtggcccagttcggccagatgatcaccggcggcagcgccgacggcagcgccgacagcaccggcagggtgcccgacaggctggccgtggtgagggtggagaccggcgagagcctgtacgacgtggccgtgagggtggcccccaacgcccccaccaggcaggtggccgacaggatcagggagctgaacggcctgcagacccccgccctggccgtgggccagaccctgatcgcccccgtgggcatgcagcagaccgcctgggcccccaggaccagcggcatcgccggctgcggcgccggcggcgtggtgatggccatcgccagcgtgaccctggtgaccgacacccccggcagggtgctgaccggcgtggccgccctgggcctgatcctgttcgccagcgccacctggagggccaggcccaggctggccatcacccccgacggcctggccatcaggggctggttcaggacccagctgctgaggcacagcaacatcaagatcatcaggatcgacgagttcaggaggtacggcaggctggtgaggctgctggagatcgagaccgtgagcggcggcctgctgatcctgagcaggtgggacctgggcaccgaccccgtggaggtgctggacgccctgaccgccgccggctacgccggcaggggccagaggatggccgtgaacaggagggtgcccagggtgagggacctggcccccctgctgcagttcaacaggccccagttcgacaccagcaagaggaggctgggcgccgccctgaccatccaggacctgaggaggatcgccaagaggaggacccccagggccgccttcgactacgccgacggcggcgccgaggacgagctgagcatcgccagggccaggcagggcttcagggacatcgagttccaccccaccatcctgagggacgtgaccaccgtgtgcgccggctggaacgtgctgggccagcccaccgtgctgcccttcggcatcgcccccaccggcttcaccaggctgatgcacaccgagggcgagatcgccggcgccagggccgccgccgccgccggcatccccttcagcctgagcaccctggccacctgcgccatcgaggacctggtgatcgccgtgccccagggcaggaagtggttccagctgtacatgtggagggacagggacaggagcatggccctggtgaggagggtggccgccgccggcttcgacaccatgctggtgaccgtggacgtgcccgtggccggcgccaggctgagggacgtgaggaacggcatgagcatcccccccgccctgaccctgaggaccgtgctggacgccatgggccaccccaggtggtggttcgacctgctgaccaccgagcccctggccttcgccagcctggacaggtggcccggcaccgtgggcgagtacctgaacaccgtgttcgaccccagcctgaccttcgacgacctggcctggatcaagagccagtggcccggcaagctggtggtgaagggcatccagaccctggacgacgccagggccgtggtggacaggggcgtggacggcatcgtgctgagcaaccacggcggcaggcagctggacagggcccccgtgcccttccacctgctgccccacgtggccagggagctgggcaagcacaccgagatcctggtggacaccggcatcatgagcggcgccgacatcgtggccgccatcgccctgggcgccaggtgcaccctgatcggcagggcctacctgtacggcctgatggccggcggcgaggccggcgtgaacagggccatcgagatcctgcagaccggcgtgatcaggaccatgaggctgctgggcgtgacctgcctggaggagctgagccccaggcacgtgacccagctgaggaggctgggccccatcggcgcccccacctacccctacgacgtgcccgactacgcctga

SEQ ID NO: 69 - protein, 729 aa, synthetic construct
  REGION 1..729: note = Rv2719c-Rv0010c-Rv1872c fusion protein
MTPVRPPHTPDPLNLRGPLDGPRWRRAEPAQSRRPGRSRPGGAPLRYHRTGVGMSRTGHGSRPVPPATTVGLALLAAAITLWLGLVAQFGQMITGGSADGSADSTGRVPDRLAVVRVETGESLYDVAVRVAPNAPTRQVADRIRELNGLQTPALAVGQTLIAPVGMQQTAWAPRTSGIAGCGAGGVVMAIASVTLVTDTPGRVLTGVAALGLILFASATWRARPRLAITPDGLAIRGWFRTQLLRHSNIKIIRIDEFRRYGRLVRLLEIETVSGGLLILSRWDLGTDPVEVLDALTAAGYAGRGQRMAVNRRVPRVRDLAPLLQFNRPQFDTSKRRLGAALTIQDLRRIAKRRTPRAAFDYADGGAEDELSIARARQGFRDIEFHPTILRDVTTVCAGWNVLGQPTVLPFGIAPTGFTRLMHTEGEIAGARAAAAAGIPFSLSTLATCAIEDLVIAVPQGRKWFQLYMWRDRDRSMALVRRVAAAGFDTMLVTVDVPVAGARLRDVRNGMSIPPALTLRTVLDAMGHPRWWFDLLTTEPLAFASLDRWPGTVGEYLNTVFDPSLTFDDLAWIKSQWPGKLVVKGIQTLDDARAVVDRGVDGIVLSNHGGRQLDRAPVPFHLLPHVARELGKHTEILVDTGIMSGADIVAAIALGARCTLIGRAYLYGLMAGGEAGVNRAIEILQTGVIRTMRLLGVTCLEELSPRHVTQLRRLGPIGAPTYPYDVPDYA

SEQ ID NO: 70 - DNA, 2079 bp, synthetic construct
  misc_feature 1..2079: note = Rv0012-Rv0990c-Rv0995 fusion protein
  source 1..2079: mol_type = other DNA
atgaggctgacccaccccaccccctgccccgagaacggcgagaccatgatcgacaggaggaggagcgcctggaggttcagcgtgcccctggtgtgcctgctggccggcctgctgctggccgccacccacggcgtgagcggcggcaccgagatcaggaggagcgacgcccccaggctggtggacctggtgaggagggcccaggccagcgtgaacaggctggccaccgagagggaggccctgaccaccaggatcgacagcgtgcacggcaggagcgtggacaccgccctggccgccatgcagaggaggagcgccaagctggccggcgtggccgccatgaaccccgtgcacggccccggcctggtggtgaccctgcaggacgcccagagggacgccaacggcaggttccccagggacgccagccccgacgacctggtggtgcaccagcaggacatcgaggccgtgctgaacgccctgtggaacgccggcgccgaggccatccagatgcaggaccagaggatcatcgccatgagcatcgccaggtgcgtgggcaacaccctgctgctgaacggcaggacctacagccccccctacaccatcgccgccatcggcgacgccgccgccatgcaggccgccctggccgccgcccccctggtgaccctgtacaagcagtacgtggtgaggttcggcctgggctactgcgaggaggtgcaccccgacctgcagatcgtgggctacgccgaccccgtgaggatgcacttcgcccagcccgccggccccctggactacgtggccgagagcagcctgaaccccagcctggtgagcaggatcagcgccttcctgaggcccgactggaccaggaccgtgagggccaggaggttcgccgccgccggcctggtgatgctggccggcgtggccgccctgaggagcaaccccgaggacgacaggagcgaggtggtggtggccgcccacgacctgaggcccggcaccgccctgacccccggcgacgtgaggctggagaagaggagcgccaccaccctgcccgacggcagccaggccgacctggacgccgtggtgggcagcaccctggccagccccaccaggaggggcgaggtgctgaccgacgtgaggctgctgggcagcaggctggccgagagcaccgccggccccgacgccaggatcgtgcccctgcacctggccgacagcgccctggtggacctggtgagggtgggcgacgtggtggacgtgctggccgcccccgtgaccgacagccccgccgccctgaggctgctggccaccgacgccatcgtggtgctggtgagcgcccagcagaaggcccaggccgccgacagcgacagggtggtgctggtggccctgcccgccaggctggccaacaccgtggccggcgccgccctgggccagaccgtgaccctgaccctgcacatggccgtgggccccctgagggtgagcgccggcgtgatcaggctgaggcccgtgaggatgagggacggcgtgcactggagcaggatcaggctggccgacagggcccacctggagccctgggagcccagcgccgacggcgagtggaccgtgaggcacaccgtggccgcctggcccgccgtgtgcagcggcctgaggagcgaggccaggaacggcaggatgctgccctacgtgatcgagctggacggccagttctgcggccagctgaccatcggcaacgtgacccacggcgccctgaggagcgcctggatcggctactgggtgcccagcgccgccaccggcggcggcgtggccaccggcgccctggccctgggcctggaccactgcttcggccccgtgatgctgcacagggtggaggccaccgtgaggcccgagaacgccgccagcagggccgtgctggccaaggtgggcttcagggaggagggcctgctgaggaggtacctggaggtggacagggcctggagggaccacctgctgatggccatcaccgtggaggaggtgtacggcagcgtggccagcaccctggtgagggccggccacgccagctggccctacccctacgacgtgcccgactacgcctga

SEQ ID NO: 71 - protein, 692 aa, synthetic construct
  REGION 1..692: note = Rv0012-Rv0990c-Rv0995 fusion protein
MRLTHPTPCPENGETMIDRRRSAWRFSVPLVCLLAGLLLAATHGVSGGTEIRRSDAPRLVDLVRRAQASVNRLATEREALTTRIDSVHGRSVDTALAAMQRRSAKLAGVAAMNPVHGPGLVVTLQDAQRDANGRFPRDASPDDLVVHQQDIEAVLNALWNAGAEAIQMQDQRIIAMSIARCVGNTLLLNGRTYSPPYTIAAIGDAAAMQAALAAAPLVTLYKQYVVRFGLGYCEEVHPDLQIVGYADPVRMHFAQPAGPLDYVAESSLNPSLVSRISAFLRPDWTRTVRARRFAAAGLVMLAGVAALRSNPEDDRSEVVVAAHDLRPGTALTPGDVRLEKRSATTLPDGSQADLDAVVGSTLASPTRRGEVLTDVRLLGSRLAESTAGPDARIVPLHLADSALVDLVRVGDVVDVLAAPVTDSPAALRLLATDAIVVLVSAQQKAQAADSDRVVLVALPARLANTVAGAALGQTVTLTLHMAVGPLRVSAGVIRLRPVRMRDGVHWSRIRLADRAHLEPWEPSADGEWTVRHTVAAWPAVCSGLRSEARNGRMLPYVIELDGQFCGQLTIGNVTHGALRSAWIGYWVPSAATGGGVATGALALGLDHCFGPVMLHRVEATVRPENAASRAVLAKVGFREEGLLRRYLEVDRAWRDHLLMAITVEEVYGSVASTLVRAGHASWPYPYDVPDYA